(19) United States
(12) Patent Application Publication
(10) Pub. No.: US 2021/0128740 A1
(43) Pub. Date: May 6, 2021

(54) HDAC INHIBITORS-BASED ANTIBODY DRUG CONJUGATES (ADCs) AND USE IN THERAPY

(71) Applicant: ALFASIGMA S.P.A., Bologna (IT)

(72) Inventors: Loredana Vesci, Roma (IT); Rita De Santis, Pomezia (RM) (IT); Ferdinando Maria Milazzo, Roma (IT); Giuseppe Giannini, Pomezia (RM) (IT); Maurizio Taddei, Monteriggioni (Siena) (IT); Valentina Faltoni, Monte San Savino (AR) (IT); Elena Petricci, Siena (IT)

(73) Assignee: ALFASIGMA S.P.A., Bologna (IT)

(21) Appl. No.: 16/492,355

(22) PCT Filed: Mar. 27, 2018
(86) PCT No.: PCT/EP2018/057744

§ 371 Date: Sep. 9, 2019

(30) Foreign Application Priority Data
Mar. 27, 2017 (EP) 17163065.0

(51) Int. Cl. A61K 47/68; C07K 16/28; C07K 16/32; A61K 9/00; A61P 35/00; A61P 35/04

(52) U.S. Cl. CPC A61K 47/6803; A61K 47/6849; C07K 16/2863; A61P 35/04; A61K 47/6889; A61K 9/0078; A61P 35/00; C07K 16/32

(57) ABSTRACT

The present invention relates to novel Histone Deacetylase Inhibitors (HDACi)-based antibody drug conjugates particularly with antibodies directed to ErbB, ErbB2 and ErbB3 receptors, pharmaceutical compositions comprising said antibodies as well as to their use in the treatment of cancer or tumor and other diseases where a modulation of one or more histone deacetylase isoforms can be effective for therapeutic interventions.

### TECHNICAL FIELD

The present invention is directed to novel Histone Deacetylase Inhibitors (HDACi)-based antibody drug conjugates useful for the treatment of proliferative diseases. The invention particularly relates to an antibody-drug-conjugate comprising an antibody directed to ErbB1, ErbB2, ErbB3 receptors or related molecular targets. The invention further provides new HDAC inhibitor drugs comprised by the antibody-drug-conjugates. Moreover, the invention relates to ADCs pharmaceutical compositions and their use in the treatment of cancer or tumors and other diseases where a modulation of one or more histone deacetylase isoforms can be effective for therapeutic interventions.

### BACKGROUND OF THE INVENTION

Medical research is focused on personalized medicine for the treatment of cancer, neurological disorders, inflammatory diseases and viral infections. Today, the knowledge about the links between genetic variations and human diseases allows a greater understanding of their etiology.

Epigenetic aberrations can contribute to the onset and progression of the above mentioned human diseases via the gain or loss of function of epigenetic regulatory proteins (Berdasco, 2013 Hum Genet 132: 359-83), because over 1,750 proteins in human cells can be post-translationally modified at lysine residues via acetylation and deacetylation (Choudhary 2009 Science 325: 834-40). Deacetylating enzymes are considered as valuable targets to treat aberrant deacetylation related to cancer but also various other diseases such neurological disorders, inflammation, viral infections and cardiovascular disorders (Minucci 2006 Nature Rev Cancer 6: 38-51; Glozak 2007 Oncogene 26: 5420-32; Zhang 2015 Med Res Rev 35: 63-84; Dinarello 2010 Mol Med 17: 333-52).

To date, few HDACi have been approved by the FDA: vorinostat (Zolinza®; Merck) for the treatment of refractory cutaneous T-cell lymphoma (CTCL) (Duvic 2007 Blood 109: 31-39); romidepsin (Istodax®; Celgene) for the treatment of CTCL and peripheral T-cell lymphoma (PTCL) (VanderMolen 2011 J Antibiot (Tokyo) 64: 525-531) and belinostat (Beleodaq®; Spectrum Pharmaceuticals) for the treatment of PTCL (West 2014 J Clin Invest 124: 30-39). In early 2015 oral panobinostat (Farydak®, Novartis) has been approved by the FDA, as combination therapy with bortezomib and dexamethasone in patients with recurrent multiple myeloma (Garnock-Jones K P (2015) Drugs. 75: 695-704). Again, in January 2015 an orally-available small-molecule benzamide HDAC1,2,3 and 10 inhibitor, chidamide (Epidaza®, Shenzhen Chipscreen Biosciences (originator) licensed to HUYA Bioscience) has been approved by the Chinese FDA for the treatment of colorectal and lung cancer, as well as for the treatment of relapsed or refractory PTCL (Ruolan Gu, (2015) Journal of Chromatography B, 1000: 181-186).

HDAC inhibitors (HDACi) are mostly studied as anticancer agents, but there is a growing body of literature ascribing HDAC enzymes to play a crucial role in other diseases such as neurological disorders, inflammatory processes and viral infections (Dinarello 2010 Cell 140: 935-950; Gray 2011 Epigenomics 3: 431-450; Giannini 2012 Future Med Chem 4: 1439-60).

WO2015/157595 describes conjugates of cysteine engineered antibodies and heterologous moiety. Among the heterologous moiety there are mentioned drugs, and among drugs, there are Histone deacetylase inhibitors (HDAC).

Choi S et al in J Control. Release 152, suppl.1, 2011, e9-e10a describes a generic HDACi conjugate with CD7 antibody, through PLGA nanoparticles. The conjugate would be able to deliver the HDAC inhibitors to human T cell, whit ovary CD7 receptor. This document does not report any specific conjugate.

Battistuzzi G et al in Current Bioactice Compounds 12, 2016, 282-288 describes four steps of synthesis of HDAC inhibitor, which are thiol derivatives. This document does not report any information on the use of this compound to obtain other pharmaceutical compounds.

Ai T. et al in Current Med Chem 2012, 19, 475 reports the use of HDACi with anti-cancer and their synergic effect. It reports HDAC inhibitor derivatives with folic acid, retinoic acid, platinum based agents, protein kinase inhibitors and Inosine monophosphate dehydrogenase inhibitors.

West A. C. et al in Oncoimmunology 1, 2012, 376 reports the use of HDAC inhibitors combination with immune-stimulating antibodies as antitumor drug.

In particular, HDACs 1, 2 and 3 primarily nuclear have been found expecially in late stage, aggressive malingnacies in tumor cells and they have correlated with poor survival rates (Gryder 2012 Future Med Chem 4: 505-24); HDAC6 primary cellular localization in the cytoplasm, regulates acetylation states and thereby the functionality of tubulin, HSP90 and other extranuclear proteins, thus suggesting its involvement in removal of misfolded proteins in cells, cell motility and metastatic potential (Clawson 2016 Ann Transl Med 4: 287).

HDAC isoforms (2, 3, 6, 9, 10) are also involved in chronic intestinal inflammation, so HDAC inhibitors in addition to apoptosis induction of tumor cells can be used for inflammatory bowel disease (Felice 2015 Aliment Pharmacol Ther 41: 26-38).

Recently, a new class of thiol-based potent pan-HDACi (Giannini 2014 J Med Chem 57: 8358-77) has been reported, wherein a potent HDAC inhibitor, ST7612AA1, was selected as drug candidate characterized by a broad spectrum of activity against human solid and haematologic malignancies.

ST7612AA1 exhibits the peculiarity to inhibit the growth of several tumors such as Ras-mutant colon carcinoma, a subset of strongly proliferating dedifferentiated colon cancer, associated with reduced patient survival; non small cell lung tumors with wild type EGFR (and mutant KRAS) and T790 EGFR mutation; ovarian with low levels of PTEN and overexpression of ErbB1 and ErbB2 or ovarian cancer without PTEN; triple-negative breast cancer (TNBC) defined by the absence of estrogen receptor, progesterone receptor and ErbB2; acute myeloid leukemia, diffuse large B cell lymphoma. Moreover, ST7612AA1 showed to modulate some transcripts involved in immune response and in key pathogenetic pathways, such NF-κB pathway and epithelial-mesenchymal transition (EMT), thus suggesting a relevant implication not only in cancer but also in the inflammatory diseases (Vesci 2015 OncoTarget 20: 5735-48).

The action of ST7612AA1 is exerted against both nuclear and cytoplasmatic HDAC isoforms of tumor cells, leading to increased transcription of e-cadherin, keratins and other typical epithelial markers and, concomitantly, down-regulation of vimentin and other genes associated to the mesenchymal phenotype. These data suggest that the treatment with ST7612AA1 may cause a “cadherin switch” and reversion of the epithelial-mesenchymal-transition (EMT) process favoring cell differentiation. The ability of cells to transdifferentiate and dedifferentiate plays a key role in invasion and metastasis by the EMT process and differentiation may be used as an additional prognostic and predictive indicator of therapeutic effective-ness. Through the inhibition of HDAC6, ST7612AA1 was also able to target non-histone HDAC substrates like, for example, TP53, alpha-tubulin or the heat shock protein 90 (HSP90) involved in DNA damage signaling, transcription factor binding, molecular homeostasis and DNA repair processes.

ST7612AA1 proved to be able to induce HIV reactivation being potentially useful for new therapies aiming at the eradication of the viral reservoirs (Badia 2015 Anti-viral Res 123: 62-9).

To improve tumor specificity and reduce toxicity, recently antibody-drug conjugates have become a clinically validated cancer therapeutic modality. While considerable strides have been made in treating hematological tumors, challenges remain in the more difficult-to-treat solid cancers.

Antibody-drug conjugates (ADC) are a rapidly growing class of cancer drugs that combine the targeting properties of mAbs with the antitumor effects of potent cytotoxic drugs (Leal M 2014 Ann NY Acad Sci 1321: 41-54).

Currently, microtubule inhibitors are clinically validated ADC payloads. Kadcyla (Trastuzumab emtansine; Genentech), Adcetris (brentuximab vedotin; Seattle Genetics), Besponsa (Inotuzumab ozogamicin; Pfizer) and Mylotarg (Gemtuzumab Ozogamicin; Pfizer) are FDA-approved ADC therapeutics, and more than 40 other ADCs have advanced to the clinic (Okeley 2014 Hematol Oncol Clin North Am 28: 13-25; Baron 2015 J Oncol Pharm Pract 21: 132-42).

The payloads currently utilized in ADCs are highly potent cytotoxic drugs, exerting their effects on critical cellular processes required for survival. Highly potent microtubule inhibitors, such as maytansine derivatives (DM1/DM4) or auristatins (MMAE/MMAF), dominate the current ADC landscape. These typically induce apoptosis in cells undergoing mitosis by causing cell cycle arrest at G2/M. More recent works show that microtubule inhibitors may also disrupt non-dividing cells in interphase. These findings provide explanation of how the microtubule inhibitors are also cytotoxic to slowly replicating or non-dividing tumor cells thus exhibiting significant toxicity. Other classes of cytotoxic drugs used in ADCs include enediynes (calicheamicin), duocarmycin derivatives, pyrrolobenzodiazepines (PBDs) and indolinobenzodiazepines, all of which target the minor groove of DNA, and quinoline alkaloids (SN-38), which inhibit topoisomerase. Thus, the majority of payloads currently utilized in ADCs are highly potent, often cytotoxic in the picomolar range, which is thought to be a requirement for ADC strategy since only a very small amount (<1%) of the ADC injected dose localizes to the tumors (Bornstein 2015 AAPS Journal 17: 525-34; Casi and Neri 2015 J Med Chem 58: 8751-61).

The majority of ADC toxicity is thought to be derived from the payload release due to linker instability. Rapidly dividing normal cells such as cells lining the digestive tract, cells in the hair follicles and myeloid cells are at risk of toxicity from released microtubule inhibitors resulting in gastrointestinal symptoms, hair loss and myelosuppression. Some key toxicities are found with different payloads. In particular, MMAE induces peripheral neuropathy and neutropenia; MMAF is associated with thrombocytopenia and ocular toxicities; DM1 causes gastrointestinal effects as well as thrombocytopenia and neutropenia, depending on the linker and consequent metabolites; ocular toxicity is the most common adverse event with DM4-conjugated ADCs; calicheamicin causes thrombocytopenia and hepatic dysfunction; and early indications from SN-38 conjugated drugs suggests neutropenia as a frequent toxicity.

A possible strategy to minimize toxicity of next generation ADCs is the selection of low toxicity payloads.

The present invention surprisingly demonstrates that antibody-drug-conjugates made of an anti-tumor antibody, conjugated to a drug with low toxicity such as an HDACi, can exert excellent efficacy in vivo. Moreover, the use of epigenetic modulators (i.e. HDACi) for ADC construction represents a first in class example in the field. Like HDAC inhibitors, other epigenetic modulators that modulate gene expression without altering the DNA base sequence such as DNA methyltransferase inhibitors (azacitidine and decitabine) could be used (Pachaiyappan 2014 Bioorg and Med Chem Lett 24: 21-32).

The antibody-drug-conjugate is further to have a good stability in blood and body fluids and excellent anti-cancer activity while having low toxicity compared to antibody-drug-conjugates of the prior art.

It is therefore an object of the present invention to provide an antibody-drug-conjugate comprising a safe anti-cancer drug conjugated to an antibody. Another object of the present invention is to provide a method for preparing said antibody-drug-conjugate (ADC).

A further object of the present invention is to provide a pharmaceutical composition comprising said antibody-drug-conjugate.

Yet another object of the present invention is to provide said antibody-drug-conjugate for use in the treatment of cancer or tumor and other diseases where a modulation of one or more histone deacetylase isoforms can be effective for therapeutic interventions.

In particular, other diseases like metabolic disorders, autism or inflammation-associated diseases such as lung injury, autoimmune disease, asthma, and type-2 diabetes that display aberrant gene expression and epigenetic regulation during their occurrence can be involved (Samanta 2017 Biochim Biophys Acta 1863: 518-28; Akhtar 2013 Plos One 8:e67813; Mei 2014 Neuron 83: 27-49). Epigenetic modifiers targeting DNA methylation and histone deacetylation enzymes could be a source to treat the pathogenesis of these diseases.

### SUMMARY OF THE INVENTION

The present invention relates to antibody-drug-conjugates of Formula (I)

D-(CU)ₘ-(S1)ₙ-L-(S2)ₒ-(CG)ₚ-Ab (Formula I)

or a pharmaceutically acceptable salt thereof, wherein
D is a cytotoxic drug (also referred to as warhead), is a histone deacetylase inhibitor drug that contains benzamide, hydroxamate, or thiol groups as the zinc binding group (ZBG),
CU is a connecting unit, which may be absent or which is selected from

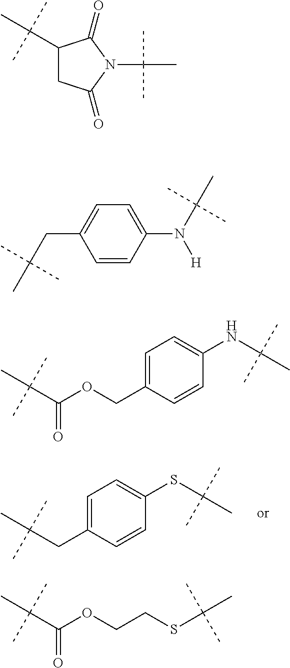

S1 is a spacer and may be absent or is

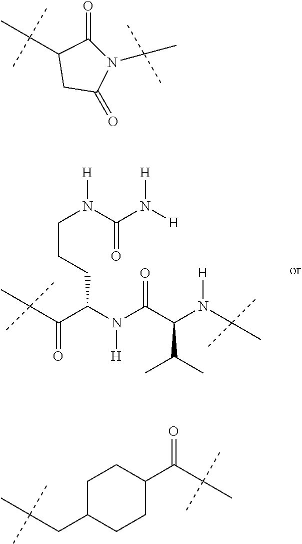

L is a linker, which is selected from (CH₂)q-CO, NH—(CH₂)r-(PEG)s-(CH₂)w-CO, NH—CO—(CH₂)r-(PEG)s-X—(CH₂)w-CO, wherein X may be absent, NH or O, q is an integer of 2-8, r may be absent or is an integer of 1-4, s may be absent or is an integer of 1-6, and w may be absent or an integer of 1-2,
S2 is a spacer and may be absent or is

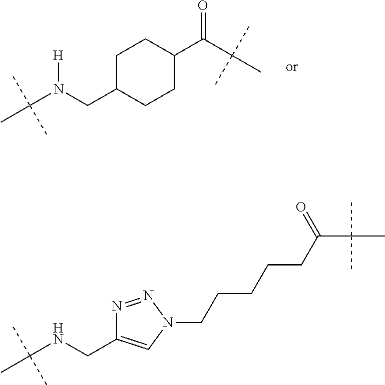

CG is a connecting group formed after conjugation to the cysteine thiol- or lysine amino-groups of the antibodies, which can be absent or one of following moieties:

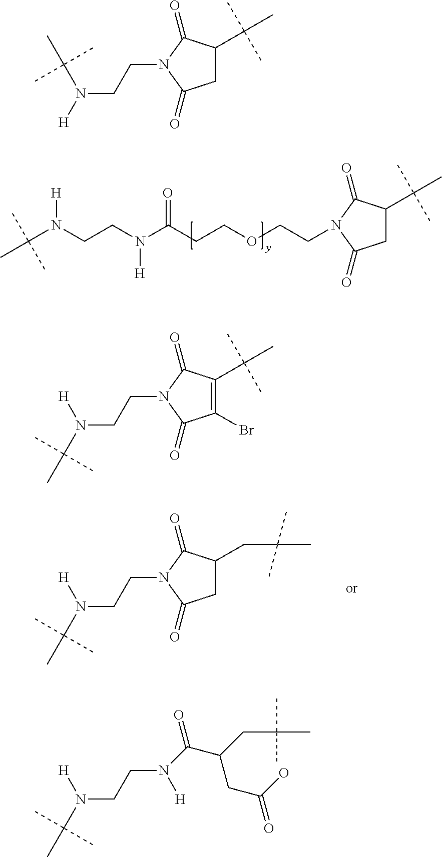

wherein y is an integer of 0 to 8
Ab is an antibody or an antigen binding fragment thereof, and
m, n, o and p represent integers of 0 or 1.

The linker (L) is preferably selected from

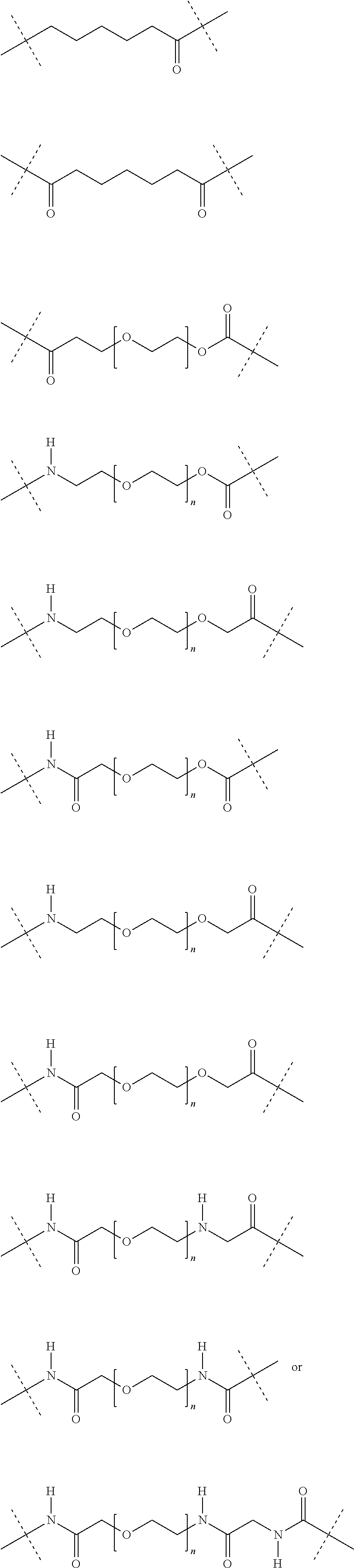

wherein n is an integer of 2-5.

In particular, a payload is a toxin (HDAC inhibitor) linked to a suitable linker/spacer, which ends with groups (i.e., maleimide, NHS esters) suitable for conjugation to antibodies and comprises the following part of formula I:

D-(CU)ₘ-(S1)ₙ-L-(S2)ₒ-(CG)′ₚ Formula II

In the present invention, (CG)′ in the payloads of Formula II, may be NHS (N-hydroxysuccinimide) or an activated acylderivative (including pentaflurophenyl ester, p-nitro and 2,4-dinitrophenol ester, thiophenol ester, acylimidazole, isobutylcarbonate, trichlorobenzoic anhydride), or maleimide- or 3-methylenesuccinimide, 3,4-dibromo maleimide or {amino-carbonyl}-3-butenoic acid of the following formulae:

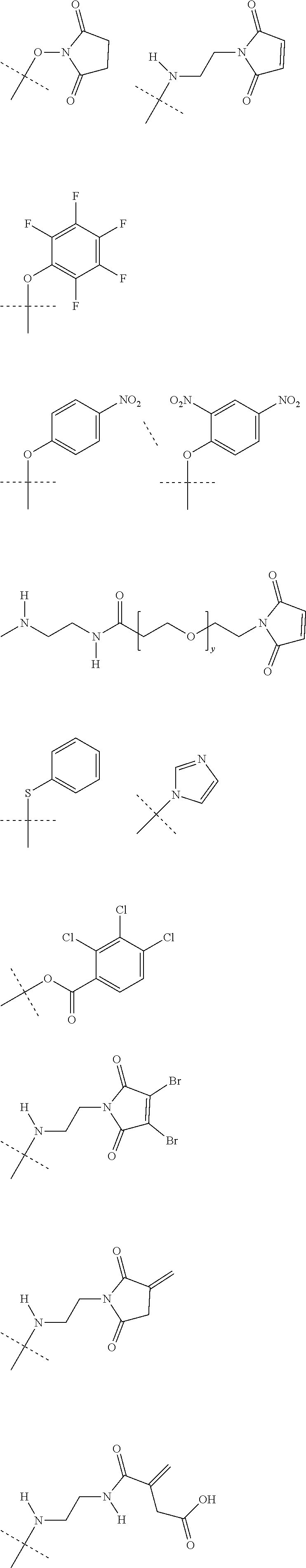

In particular, the present invention relates to ADCs having as warhead an HDAC inhibitor, is selected from thiol-based histone deacetylase inhibitors, such as ST7464AA1 and ST766AA1 (a thiol analogue of vorinostat), having the following formulas:

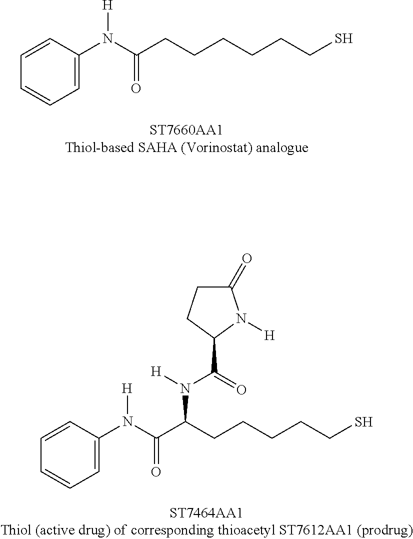 [SMILES: O=C(CCCCCCS)Nc1ccccc1 ; O=C1CC[C@H](C(=O)N[C@@H](CCCCCS)C(=O)Nc2ccccc2)N1]

or from hydroxamic acid based histone deacetylase inhibitors (HDACs), such as vorinostat (SAHA), panobinostat (LBH589), or dacinostat (LAQ824) having the following formulas:

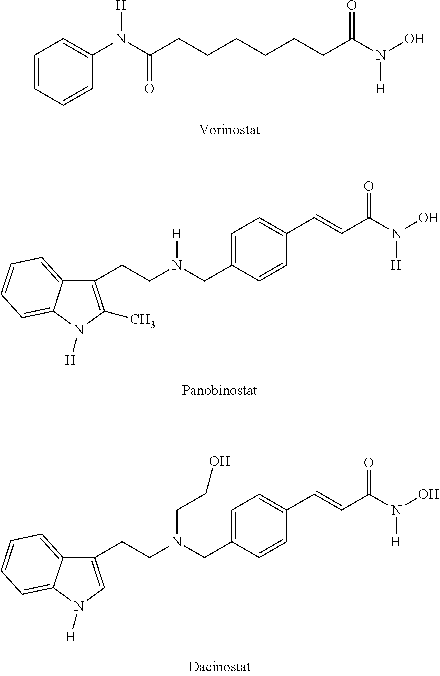 [SMILES: O=C(CCCCCCC(=O)Nc1ccccc1)NO ; Cc1[nH]c2ccccc2c1CCNCc1ccc(/C=C/C(=O)NO)cc1 ; O=C(/C=C/c1ccc(CN(CCO)CCc2c[nH]c3ccccc23)cc1)NO]

or from benzamide based histone deacetylase inhibitors (HDACs) such as entinostat (MS275), having the following formula:

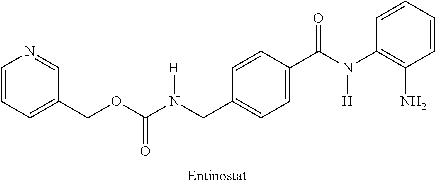 [SMILES: Nc1ccccc1NC(=O)c1ccc(CNC(=O)OCc2cccnc2)cc1]

The histone deacetylase inhibitor and the payload comprising D-(CU)ₘ-(S1)ₙ-L-(S2)-(CG)′ₚ- (Formula II) may be a compound selected from:

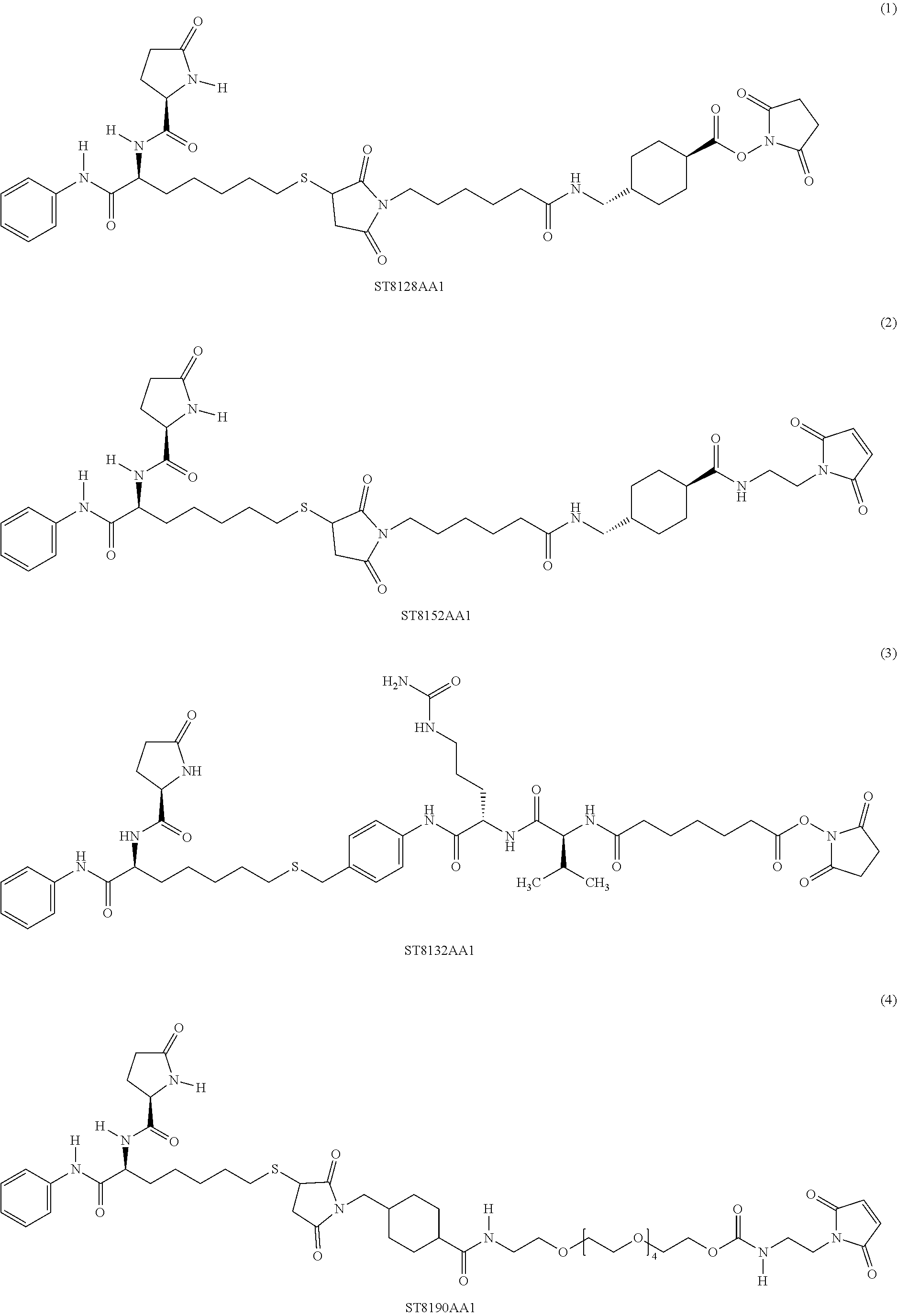

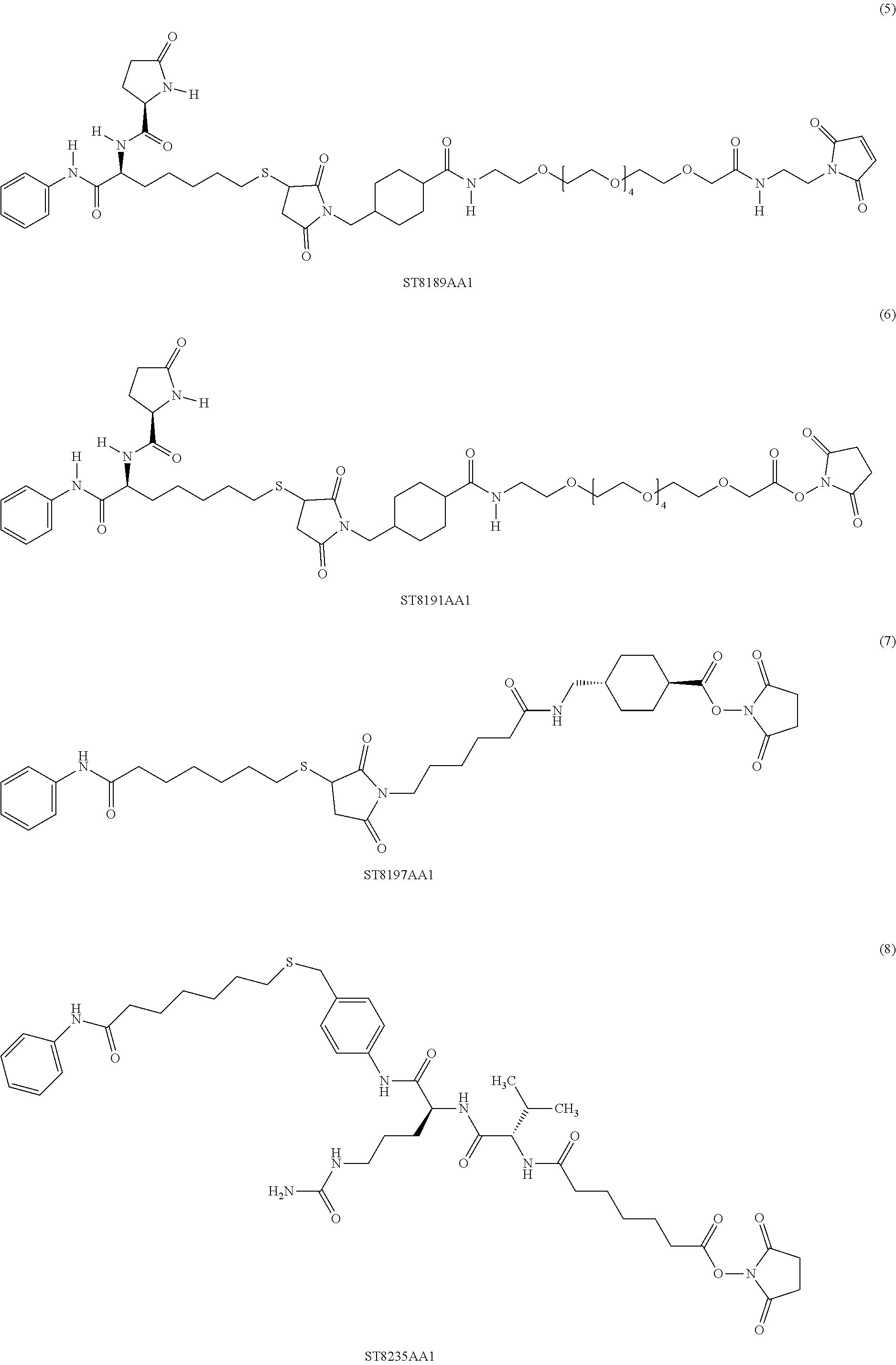

In a further embodiment, the invention relates to antibody-drug-conjugates, wherein the histone deacetylase inhibitor drug and the payload comprising the structure D-(CU)ₘ-(S1)ₙ-L-(S2)-(CG)′ₚ are selected from:

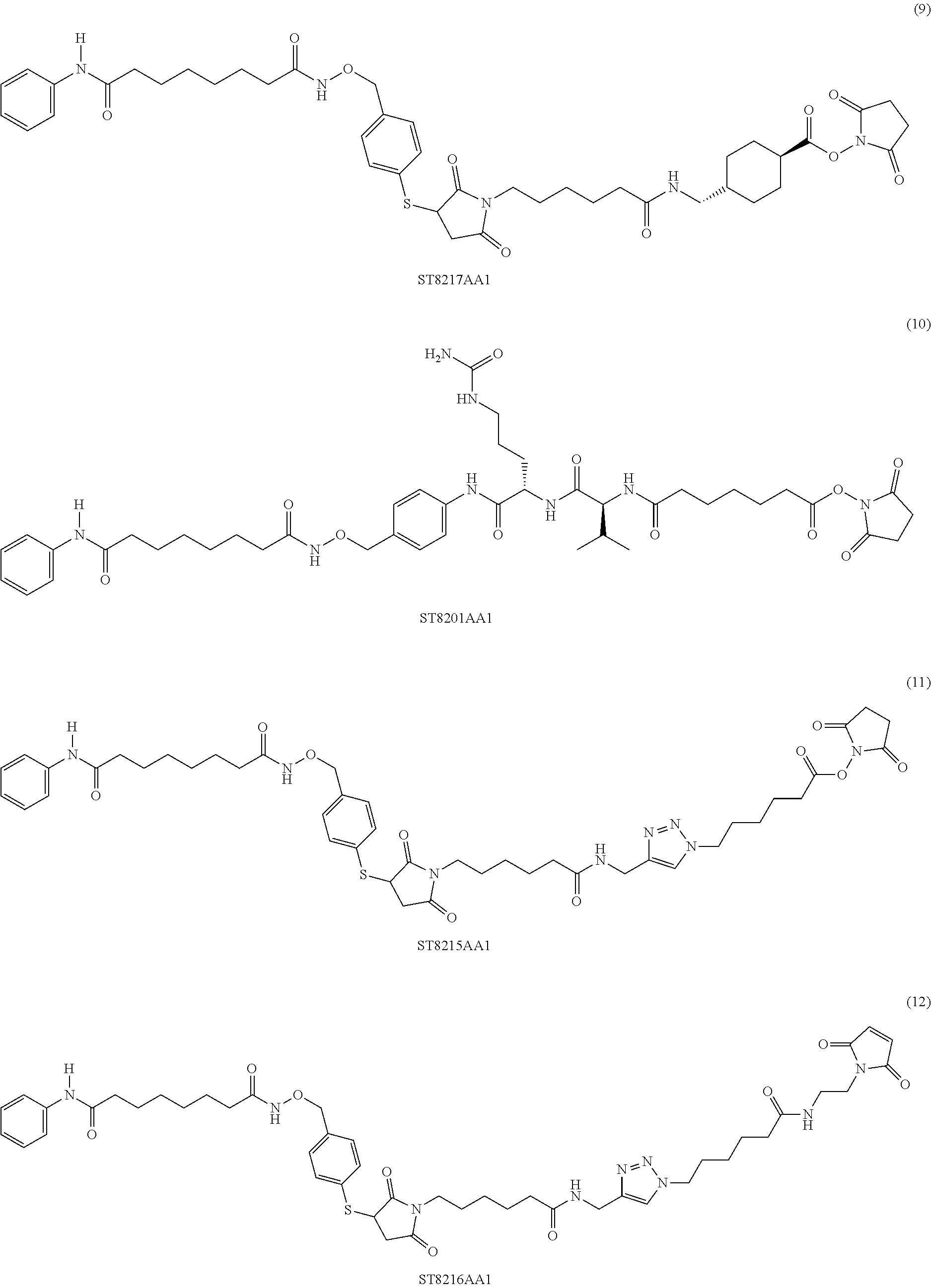

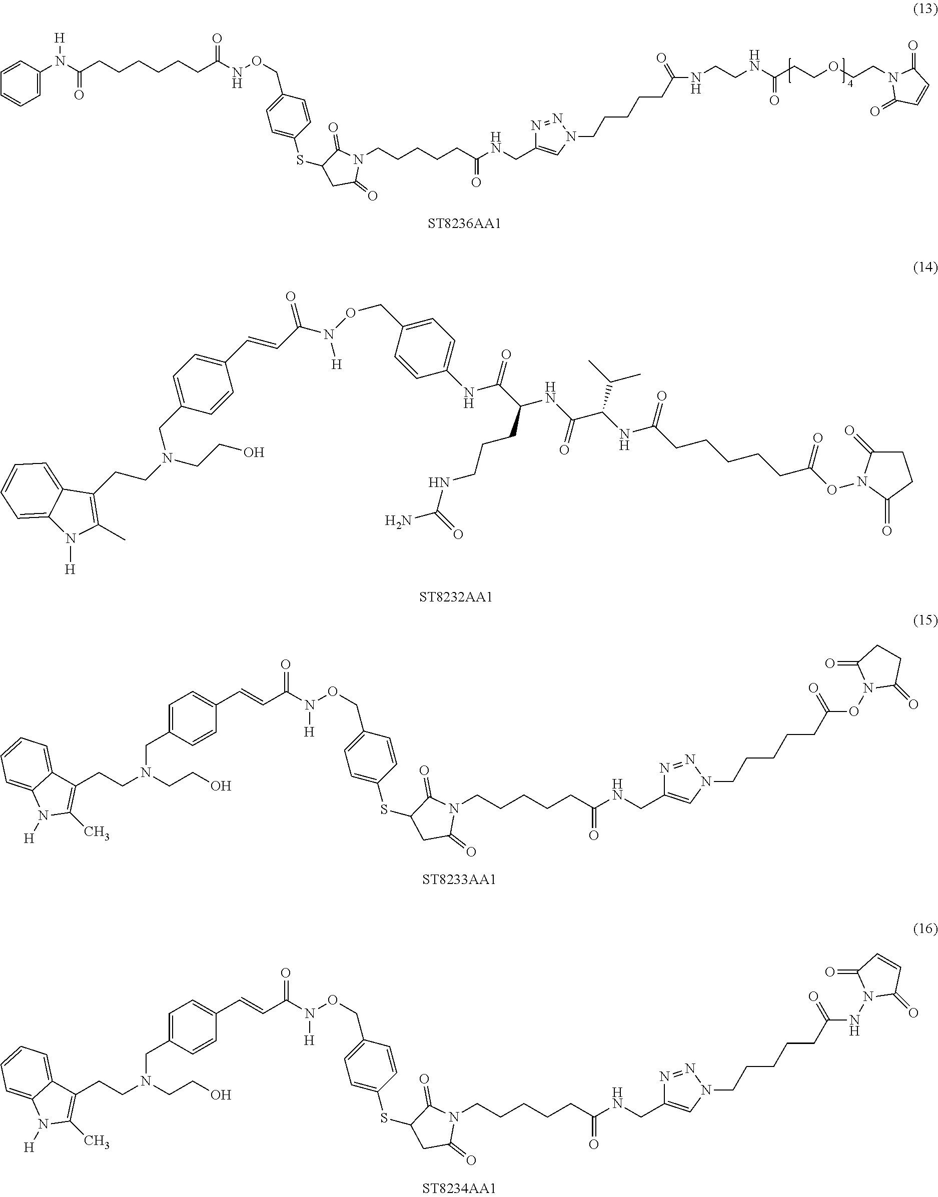

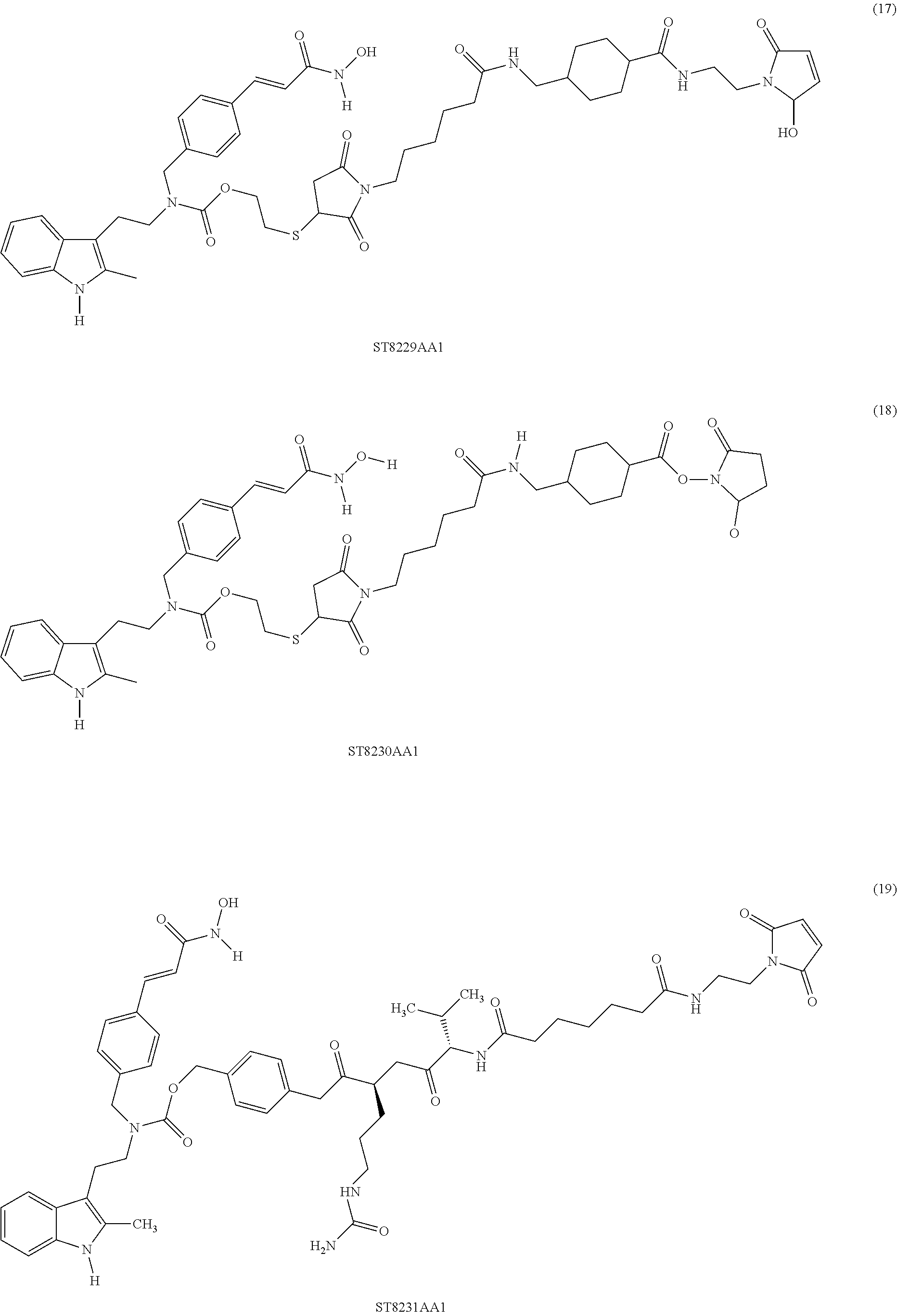 [SMILES: Cc1[nH]c2ccccc2c1CCN(Cc1ccc(/C=C/C(=O)NO)cc1)C(=O)OCCSC1CC(=O)N(CCCCCC(=O)NCC2CCC(C(=O)NCCN3C(=O)C=CC3O)CC2)C1=O ; Cc1[nH]c2ccccc2c1CCN(Cc1ccc(/C=C/C(=O)NO)cc1)C(=O)OCCSC1CC(=O)N(CCCCCC(=O)NCC2CCC(C(=O)ON3C(=O)CCC3O)CC2)C1=O ; Cc1[nH]c2ccccc2c1CCN(Cc1ccc(/C=C/C(=O)NO)cc1)C(=O)OCc1ccc(CC(=O)[C@H](CCCNC(N)=O)CC(=O)[C@@H](NC(=O)CCCCCC(=O)NCCN2C(=O)C=CC2=O)C(C)C)cc1]

In a further embodiment, the invention relates to payload-drug-conjugates, wherein payload-drug conjugate is selected from:

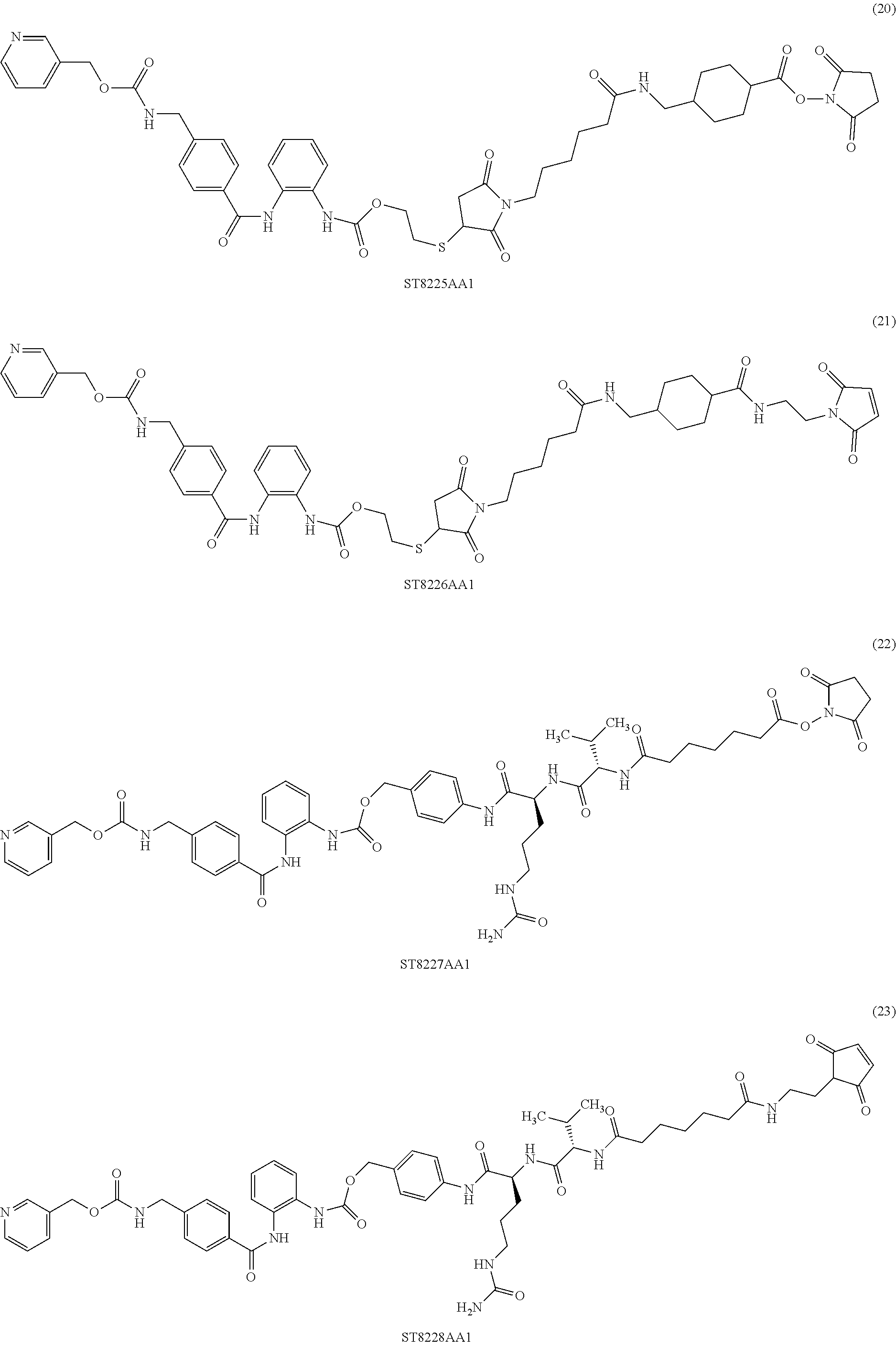 [SMILES: O=C(CCCCCN1C(=O)CC(SCCOC(=O)Nc2ccccc2NC(=O)c2ccc(CNC(=O)OCc3cccnc3)cc2)C1=O)NCC1CCC(C(=O)ON2C(=O)CCC2=O)CC1 ; O=C(CCCCCN1C(=O)CC(SCCOC(=O)Nc2ccccc2NC(=O)c2ccc(CNC(=O)OCc3cccnc3)cc2)C1=O)NCC1CCC(C(=O)NCCN2C(=O)C=CC2=O)CC1 ; CC(C)[C@H](NC(=O)CCCCCC(=O)ON1C(=O)CCC1=O)C(=O)N[C@@H](CCCNC(N)=O)C(=O)Nc1ccc(COC(=O)Nc2ccccc2NC(=O)c2ccc(CNC(=O)OCc3cccnc3)cc2)cc1 ; CC(C)[C@H](NC(=O)CCCCCC(=O)NCCC1C(=O)C=CC1=O)C(=O)N[C@@H](CCCNC(N)=O)C(=O)Nc1ccc(COC(=O)Nc2ccccc2NC(=O)c2ccc(CNC(=O)OCc3cccnc3)cc2)cc1]

In a further embodiment of invention, the antibody-drug-conjugates (ADO) derived from the compounds of 1-23.

In a further embodiment, the ADC are selected from the compounds represented by the formulas 24-37:

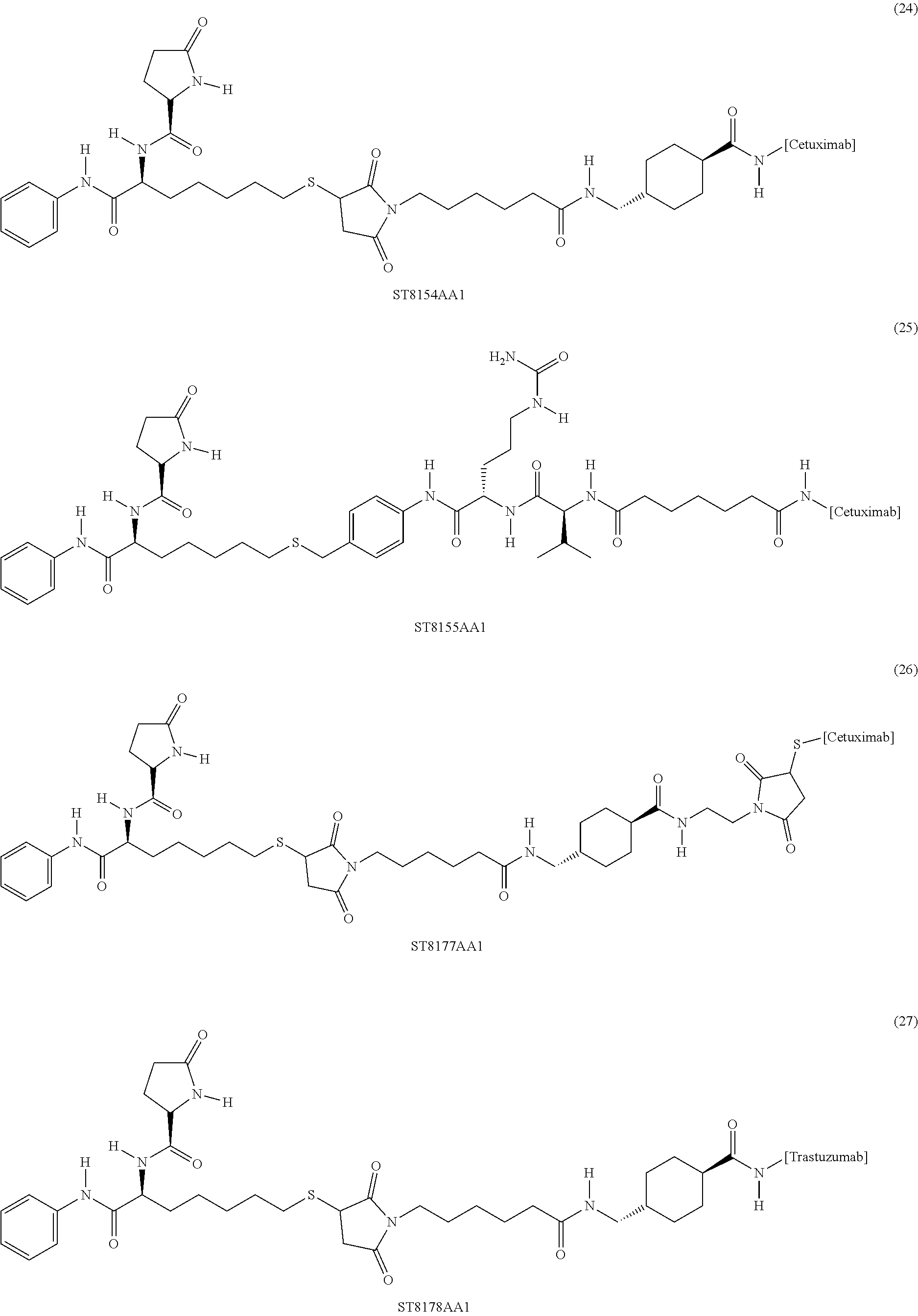

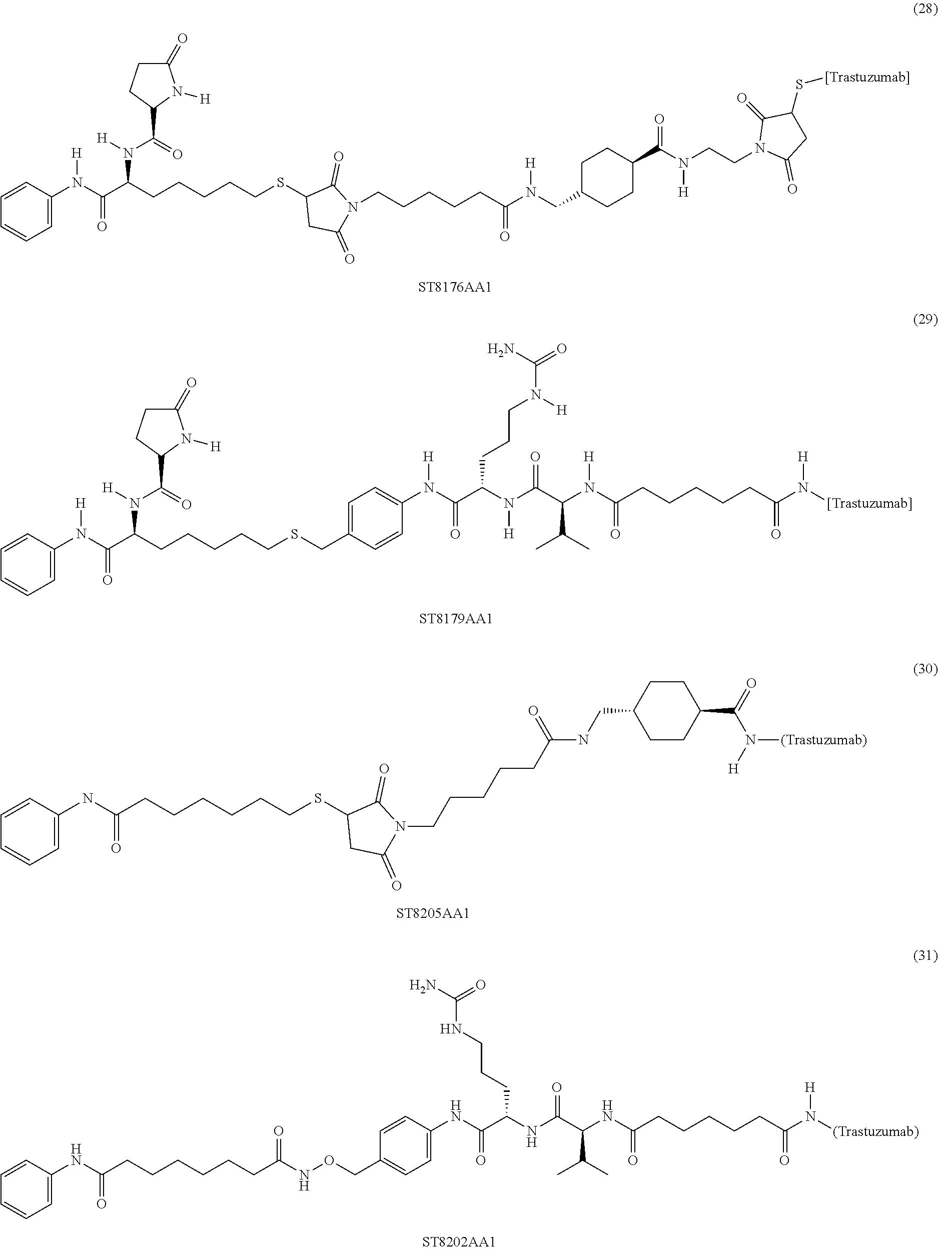

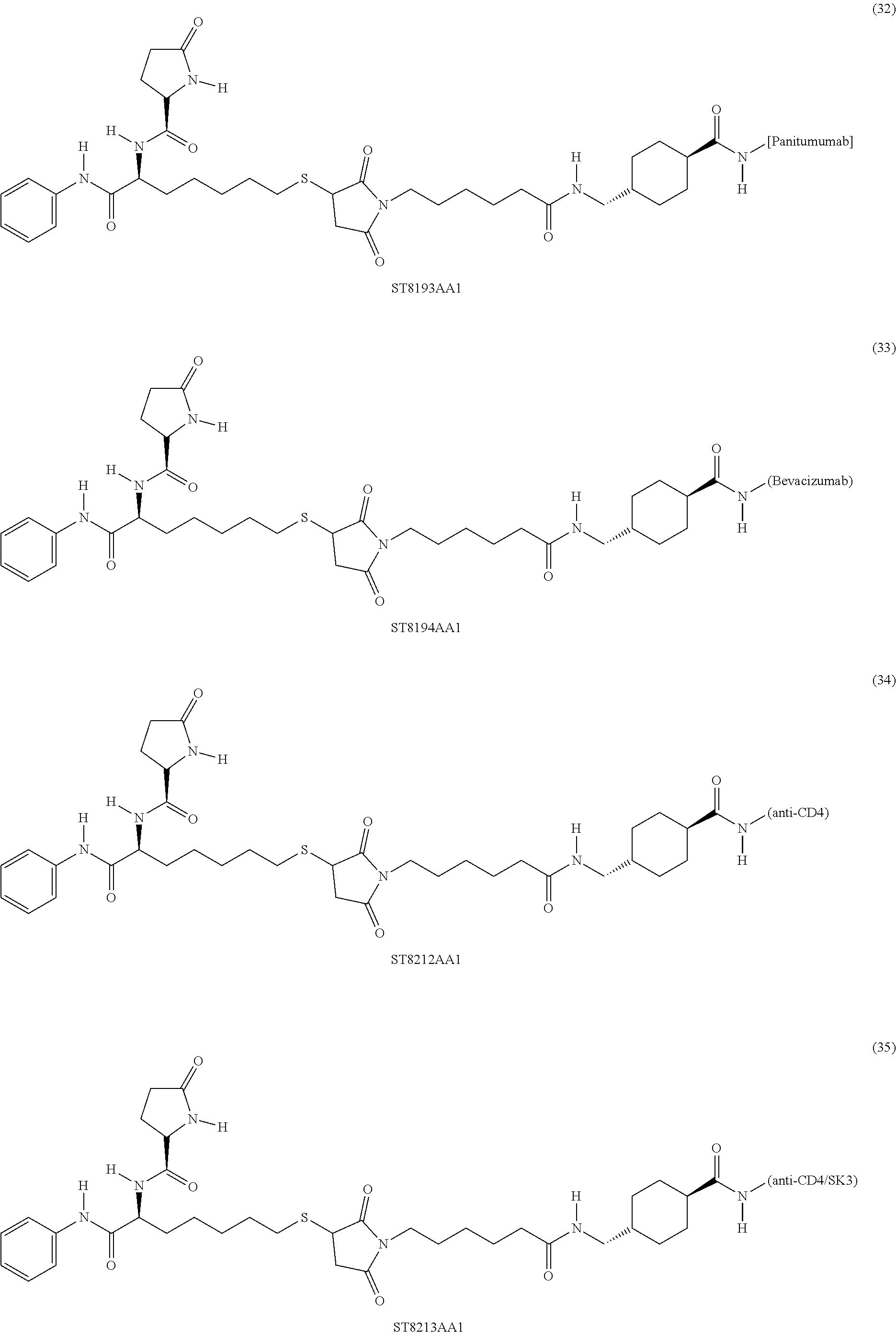

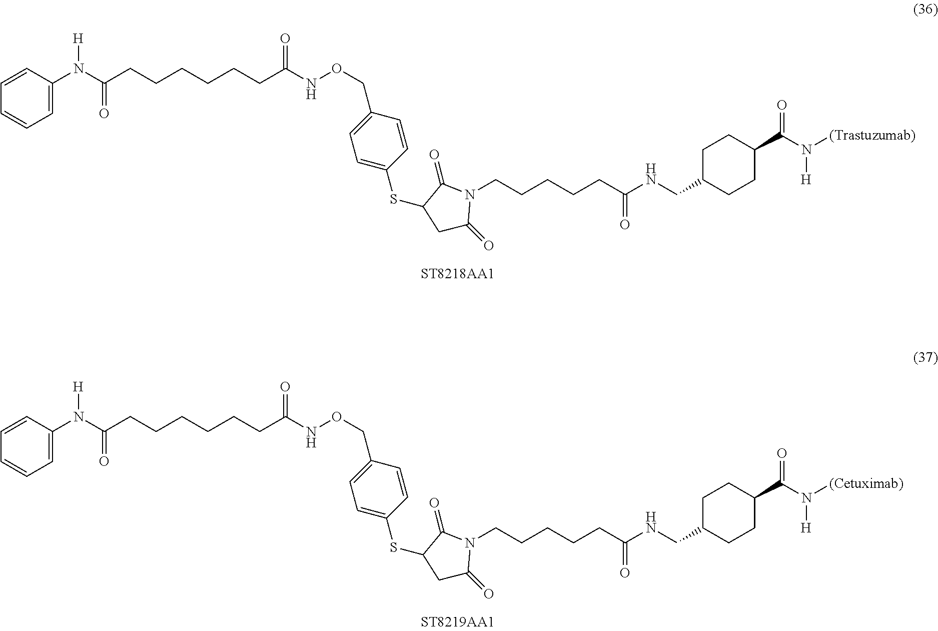

Particularly preferred are the ADCs of formulas 24 to 29.

Further embodiments of the invention are other ADCs where mAb may be one selected from those indicated in the present invention.

The present invention further provides a pharmaceutical composition comprising a therapeutically effective amount of a derivative of Formula I with pharmaceutically acceptable excipients.

Pharmaceutical composition may be for enteral or parenteral administration, wherein the enteral administration comprises oral, aerosol, rectal or buccal route, and parenteral administration comprises subcutaneous, intramuscular or intravenous, and intradermic route.

The present invention also provides a pharmaceutical composition comprising a therapeutically effective amount of a derivative of Formula I in combination with other known anticancer treatments such as radiation therapy or chemotherapy regiment, in combination with cytostatic or cytotoxic agents, antibiotic-type agents, alkylating agents, antimetabolite agents, hormonal agents, interferon type agents, cyclooxygenase inhibitors (e.g. COX-2 inhibitors), metalloproteinase inhibitors, telomerase inhibitors, tyrosine kinase inhibitors, anti-grow factor receptor agents, anti-HER2 agents, anti-EGFR agents, anti-angiogenesis agents (e.g. angiogenesis inhibitors), farnesyl transferase inhibitors, ras-raf signal transduction pathway inhibitors, cell cycle inhibitors, other cdks inhibitors, tubulin binding agents, topoisomerase I inhibitors, topoisomerase II inhibitors, and the like.

Additionally, the invention provides a product comprising a derivative of Formula (I) or a pharmaceutically acceptable salt thereof, as defined above, and one or more chemotherapeutic agents, as a combined preparation for simultaneous, separate or sequential use in anticancer therapy.

In yet another aspect the invention provides a derivative of Formula (I) or a pharmaceutically acceptable salt thereof, as defined above, for use as a medicament.

The present invention moreover relates to the antibody-drug-conjugates or a pharmaceutical composition comprising the drug antibody conjugates of the invention for use in the treatment of cancer or tumour. The invention particularly relates to the treatment of a cancer or tumour expressing ErbB1, ErbB2 and/or ErbB3 receptors. Specific examples of the cancer to be treated according to the present invention are carcinomas, including bladder, breast, colon, kidney, liver, lung, comprising small cell lung cancer, oesophagus, gall-bladder, ovary, pancreas, stomach, cervix, thyroid, prostate, and skin carcinoma, comprising squamous cell carcinoma; hematopoietic tumours of lymphoid lineage, including leukaemia, acute lymphocytic leukaemia, acute lymphoblastic leukaemia, B-cell lymphoma, T-cell lymphoma, Hodgkin's lymphoma, non-Hodgkin's lymphoma, hairy cell lymphoma and Burkitt's lymphoma; hematopoietic tumours of myeloid lineage, including acute and chronic myelogenous leukaemia, myelodysplastic syndrome and promyelocytic leukaemia; tumours of mesenchymal origin, including fibrosarcoma and rhabdomyosarcoma; tumours of the central and peripheral nervous system, including astrocytoma, neuroblastoma, glioma and schwannoma; and other tumours, including melanoma, seminoma, teratocarcinoma, osteosarcoma, xeroderma pigmentosa, keratoxanthoma, thyroid follicular cancer, Kaposi's sarcoma and mesothelioma. In addition, the HIV reactivation induced by the HDACi-based ADCs can be potentially useful for new therapies aiming at the eradication of the viral reservoirs (Badia 2015 Antiviral Res 123: 62-9). In this regard, the above described antibody-drug-conjugates may be used as an adjuvant therapeutic in the treatment of HIV.

Moreover, the invention relates to a pharmaceutical composition comprising said antibody-drug-conjugates as well as to said antibody-drug-conjugates or said pharmaceutical composition for use in the treatment of a cancer or a tumor expressing a receptor selected from ErbB1, ErbB2 or ErbB3.

### Advantageous Effects

The antibody-drug-conjugates of the present invention were more potent than the single antibodies and the cytotoxic agent, given at the same concentration, route and schedule. Moreover, it was surprising that the cytotoxic agent such as an HDAC inhibitor conjugated to the antibodies, at suboptimal dosages, resulted very effective, not depending on the linkers or type conjugation (lysine or cysteine). Subsequently, these HDAC inhibitors-based ADCs allow obtaining antitumor efficacy at lower dosages than the corresponding antibodies, thus resulting in minor toxicity.

### BRIEF DESCRIPTION OF THE DRAWINGS

FIG. 1 shows MALDI mass spectra of Cetuximab (up) and of its conjugated forms ST8154AA1 (24) with payload-NHS ST8128AA1 (1) (down). The DAR calculated from the mass difference was 8.9.

FIG. 2 shows MALDI mass spectra of Trastuzumab (up) and of its conjugated forms ST8178AA1 (27) with payload-NHS ST8128AA1 (1) (down). The DAR calculated from the mass difference was 6.9.

FIG. 3 shows binding (FACS analysis) of native Cetuximab and Cetuximab-derived ADCs, ST8154AA1 (24) and ST8177AA1 (26) (A) or ST8219AA1 (37) (B), to different tumor cell lines. Antibody binding detected by FITC-conjugated mouse anti-human Ig (BD). Grey peaks refer to cells without primary antibody.

FIG. 4 shows binding (FACS analysis) of native Trastuzumab and Trastuzumab-derived ADCs, ST8178AA1 (27) and ST8176AA1 (28) (A), or ST8205AA1 (30) and ST8218AA1 (36) (B), to different tumor cell lines. Antibody binding detected by FITC-conjugated mouse anti-human Ig (BD). Grey peaks refer to cells without primary antibody.

FIG. 5 shows immunoreactivity of ADCs, tested by antigen-specific ELISA. Activity measured against A) recombinant human EGF-R/Erb1 Fc chimera, or B) recombinant Human ErbB2/HER2 protein. Detection through anti-human K light chain horseradish peroxidase (HRP)-conjugated antibody and TMB substrate addition. Optical density at 450 nm measured by ELISA spectrophotometer. Results are the mean (±SD) of two independent replicates.

FIG. 6 shows antiproliferative activity of ST8154AA1 (24) on NCI-H1975 non-small cell lung carcinoma cells upon 6 days of treatment. IC50 value±SD of the ADC was 250±10 nM, in comparison with Cetuximab, which was ineffective (IC50>500 nM).

FIG. 7 shows antiproliferative activity of ST8154AA1 (24) on Calu-3 non-small cell lung carcinoma cells upon 6 days of treatment. IC50 value±SD of the ADC was 450±10 nM, in comparison with Cetuximab, which was ineffective (IC50>500 nM).

FIG. 8 shows the effect of Cetuximab-derived ADCs ST8154AA1 (24) and ST8177AA1 (26) (A), or ST8219AA1 (37) (B), on the level of acetylated-α-tubulin in different tumor cell lines. Cells were cultivated 3 hours at 37° C. with antibodies (5 μg/mL). Following two washings, cells were then fixed and stained with mouse anti-acetylated-α-tubulin IgG and then with FITC-conjugated goat anti-mouse IgG. Draq5 dye staining of nucleus and cytoplasm. Insets show fluorescence signals specifically associated to acetylated-α-tubulin. Fluorescence imaging by High Content Screening (HCS) Operetta. Each image is representative of at least 5 fields of duplicate wells. Magnification 60×. Data are from one representative experiment out of two.

FIG. 9a and FIG. 9b show the effect of Trastuzumab-derived ADCs ST8178AA1 (27) and ST8176AA1 (28) (A), or ST8202AA1 (31), ST8205AA1 (30) and ST8218AA1 (36) (B), on the level of acetylated-α-tubulin in different tumor cell lines. Cells were cultivated 3 hours at 37° C. with antibodies (5 μg/mL). Following two washings, cells were then fixed and stained with mouse anti-acetylated-α-tubulin IgG and then with FITC-conjugated goat anti-mouse IgG. Draq5 dye staining of nucleus and cytoplasm. Insets show fluorescence signals specifically associated to acetylated-α-tubulin. Fluorescence imaging by High Content Screening (HCS) Operetta. Each image is representative of at least 5 fields of duplicate wells. Magnification 60×. Data are from one representative experiment out of two.

FIG. 10 shows the effect of Cetuximab-derived ADCs ST8154AA1 (24) and ST8177AA1 (26) (A), or ST8219AA1 (37) (B), on the level of acetylated-histone H3 in different tumor cell lines. Cells were cultivated 3 hours at 37° C. with antibodies (5 μg/mL). Following two washings, cells were then fixed and stained with rabbit anti-acetylated-histone H3 IgG and then with FITC-conjugated goat anti-rabbit IgG. Draq5 dye staining of nucleus and cytoplasm. Insets show fluorescence signals specifically associated to acetylated-histone H3. Fluorescence imaging by High Content Screening (HCS) Operetta. Each image is representative of at least 5 fields of duplicate wells. Magnification 60×. Data are from one representative experiment out of two.

FIG. 11 shows the effect of Trastuzumab-derived ADCs ST8178AA1 (27) and ST8176AA1 (28) (A), or ST8202AA1 (31), ST8205AA1 (30) and ST8218AA1 (36) (B), on the level of acetylated-histone H3 in different tumor cell lines. Cells were cultivated 3 hours at 37° C. with antibodies (5 μg/mL). Following two washings, cells were then fixed and stained with rabbit anti-acetylated-histone H3 IgG and then with FITC-conjugated goat anti-rabbit IgG. Draq5 dye staining of nucleus and cytoplasm. Insets show fluorescence signals specifically associated to acetylated-α-histone H3. Fluorescence imaging by High Content Screening (HCS) Operetta. Each image is representative of at least 5 fields of duplicate wells. Magnification 60×. Data are from one representative experiment out of two.

FIG. 12 shows the effect of ADCs on acetylation of α-tubulin and histone H4 in A549 (A) and SKBR3 (B) cell lines. Cells were cultivated 3 hours at 37° C. with antibodies (20 μg/mL) and then Western Blot analysis was carried out on total protein lysates. Representative blots are shown.

FIG. 13 shows the antitumor activity of ST8155AA1 (25), ST8154AA1 (24), and ST7612AA1 given intraperitoneally according to the schedule q4dx4, in comparison with Cetuximab in sc NCI-H1975 tumor bearing mice. Tumor cells (5×10⁶). were sc injected in the right flank of mice The ADCs and Cetuximab were given at a dose of 50 mg/kg, whereas ST7612AA1 at 120 mg/kg. Tumor lesions were evaluated by a digital caliper (n=8 mice/group; mean and SEM, ° ° ° P<0.001 and ° ° P<0.01 vs Cetuximab alone, Mann-Whitney's test).

FIG. 14 shows the antitumor effect of the ADC ST8154AA1 (24), in comparison with Cetuximab, in tumors developed in Nu/Nu mice after s.c. injection of 5×10⁶ A549 cells. Lesion development and response to antibody treatment was monitored using a digital caliper. Mice injected i.p. with either (24) and Cetuximab (4 doses of 50 mg/kg once every 4 days) or PBS (n=10 mice/group; mean and SEM, ° P<0.05 vs Cetuximab; **P<0.01 and *P<0.05 vs vehicle, Mann-Whitney's test).

FIG. 15 shows the antimetastatic activity of ST8154AA1 (24) in comparison with Cetuximab (Ctx) on artificial metastatic lung cancer resulting from the injection of 5×10⁶ A549-luc-C8 (A549luc) cells into the tail vein of immunodeficient SCID/beige mice. Tumor bioluminescence imaging (BLI) was recorded by Xenogen IVIS Imaging System 200, at different time points (+35, +49 and +56 days from cell injection), after i.p. injection of luciferin (150 μg/mouse). Mice were treated by aerosol with PBS or ST8154AA1 or Ctx (3.5 mL of 100 μg/mL solution) q7dx4 (n=12 mice/group; mean and SEM, ° ° P<0.01 vs Ctx; *P<0.05 and **P<0.01 vs vehicle).

FIG. 16 shows the antitumor effect of ST8154AA1 (24) in comparison with Cetuximab (Ctx) and ST7612AA1 against an orthotopic tumor pancreas. Tumor cells 1×10⁶ were injected directly into pancreas. Tumor weight was evaluated 90 days after tumor injection. Mice were treated intraperitoneally with (24) or Ctx (4 doses of 40 mg/kg once every 4 days), PBS and ST7612AA1 (200 mg/kg, q4dx4) (n=10 mice/group); mean and SEM, ° P<0.05 vs Ctx,*P<0.05 and **P<0.01 vs vehicle.

FIG. 17 shows the antitumor activity of ST8154AA1 (24) in comparison with Cetuximab delivered intraperitoneally (q4dx5) against a patient-derived tumor xenograft (PDX) pancreas carcinoma implanted sc in nude mice. NOD SCID mice received sc tumor cells (51000 cells) from a patient PA5363. Treatment of tumor bearing mice was carried out with the ADC at a dose of 40 mg/kg, ip. Tumor growth was evaluated by a digital caliper (n=10 mice/group). Mean and SEM, ° P<0.05 vs Cetuximab, ***P<0.001 vs vehicle.

FIG. 18 shows the antitumor activity of ST8178AA1 (27) in comparison with Trastuzumab delivered intraperitoneally according to the schedule q4dx4 against SKOV-3 ovarian carcinoma. Tumors were allowed to develop in Nu/Nu mice after s.c. injection of 5×10⁶ SKOV-3 ovarian cells. Treatment was carried out with the ADC and Trastuzumab at 15 mg/kg, ip (n=11 mice/group; mean and SEM, *P<0.05 vs vehicle, ° P<0.05 vs Trastuzumab, Mann-Whitney's test).

FIG. 19 shows the antitumor activity of ST8176AA1 (28) in comparison with Trastuzumab delivered intraperitoneally according to the schedule q4dx4 against SKOV-3 ovarian carcinoma. Tumors were allowed to develop in Nu/Nu mice after s.c. injection of 5×10⁶ SKOV-3 ovarian cells. Treatment was carried out with the ADC and Trastuzumab at 30 and 15 mg/kg, ip (n=12 mice/group; mean and SEM, *P<0.05 vs vehicle, ° P<0.05 vs Trastuzumab, Mann-Whitney's test).

FIG. 20 shows the antitumor activity of ST8176AA1 (28) in comparison with Trastuzumab delivered intraperitoneally according to the schedule q4dx4 against SKOV-3 ovarian carcinoma. Tumors were allowed to develop in Nu/Nu mice after i.p. injection of 10×10⁶ SKOV-3 ovarian cells. Treatment was carried out with the ADC and Trastuzumab at 15 mg/kg, ip (n=9 mice/group). Survival curve was plotted by Kaplan-Meier analysis. *P<0.05 and **P<0.01 vs vehicle, ° P<0.05 vs Trastuzumab.

FIG. 21 shows the antitumor activity of ST8176AA1 (28) in comparison with Trastuzumab delivered intraperitoneally according to the schedule q4dx4 against LS174-T colon carcinoma. Tumors were allowed to develop in Nu/Nu mice after i.p. injection of 10×10⁶ LS174T colon cancer cells. Treatment was carried out with the ADC and Trastuzumab at 15 mg/kg, ip (n=10 mice/group). Survival curve was plotted by Kaplan-Meier analysis. P values were calculated using a two-sides log rank-test, *P<0.05 vs Vehicle and ° P<0.05 vs Trastuzumab.

FIG. 22 shows the antitumor activity of ST8176AA1 (28) in comparison with Trastuzumab delivered intraperitoneally according to the schedule q4dx4 against LS-174T colon carcinoma. Tumors were allowed to develop in Nu/Nu mice after s.c. injection of 5×10⁶ LS174-T colon cancer cells. Treatment was carried out with the ADC and Trastuzumab at 15 mg/kg, ip (n=10 mice/group; mean and SEM, *p<0.05 vs vehicle, ° P<0.05 vs Trastuzumab, Mann-Whitney's test).

FIG. 23 shows the antitumor activity of ST8176AA1 (28) in comparison with Trastuzumab delivered intraperitoneally according to the schedule q4dx4 against a PDX (patient-derived xenograft) pancreas cancer. Human pancreas tumor cells (77×10³) from the patient PA5363 were sc injected in NOD-SCID mice. Treatment was carried out with ST8176AA1 or Trastuzumab at 15 mg/kg. (n=10 mice/group; mean and SEM, *P<0.05 vs vehicle and ° P<0.05 vs Trastuzumab, Mann-Whitney's test).

### DETAILED DESCRIPTION OF THE INVENTION

The invention relates to novel ADCs made of anti-cancer antibodies conjugated to HDACi-based payloads. Such ADCs are shown to specifically bind tumor receptors, to be internalized and delivered to lysosomes. These properties surprisingly correlate with in vitro cytotoxicity and in vivo antitumor activities despite the low potency of the HDAC payloads. The antibody-drug-conjugates of the invention are particularly useful in the treatment of tumors or any other diseases where a modulation of one or more histone deacetylase isoforms and the expression of ErbB receptors are effective for therapeutic intervention.

The present invention describes safe and efficacy ADC comprising a safe HDACi conjugated with a linker to an antibody and in particular an immunoglobulin used for cancer treatment. The epigenetic modulator, HDAC inhibitor allows ADC construction with reduced negative and toxic effects.

A preferred embodiment of the present invention is the use of HDACi-based ADCs for the therapy of cancer expressing receptors such as ErbB1, ErbB2 or ErbB3 including as example, lung, breast, colon, brain, head and neck, endometrial, renal, pancreatic, gastric, oesophageal, ovarian and prostate cancer and leukaemia.

The present invention relates to antibody-drug-conjugates of Formula (I)

D-(CU)ₘ-(S1)ₙ-L-(S2)ₒ-(CG)ₚ-Ab (Formula I)
or a pharmaceutically acceptable salt thereof, wherein
D is a cytotoxic drug (also referred to as warhead), is a histone deacetylase inhibitor drug that contains benzamide, hydroxamate, or thiol groups as the zinc binding group (ZBG),
CU is a connecting unit, which may be absent or which is selected from

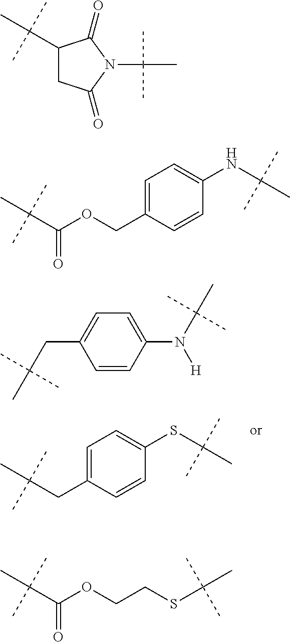

S1 is a spacer and may be absent or is

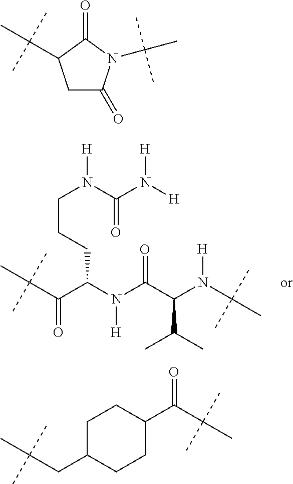

S2 is a spacer and may be absent or is
L is a linker, which is selected from (CH₂)q-CO, NH—(CH₂)r-(PEG)s-(CH₂)w-CO, NH—CO—(CH₂)r-(PEG)s-X—(CH₂)w-CO, wherein X may be absent, NH or O, q is an integer of 2 to 8, r may be absent or an integer of 1 to 4, s may be absent or an integer of 1 to 6, and w may be absent or an integer of 1 to 2,
S2 is a spacer and may be absent or is

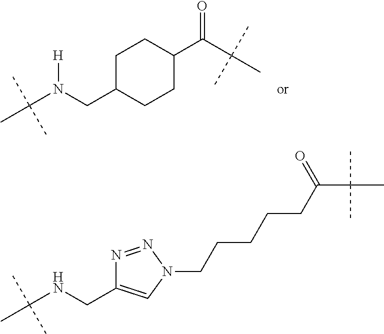

CG is a connecting group formed after conjugation to the cysteine thiol- or lysine amino-groups of the antibodies, which can be absent or one of following moieties:

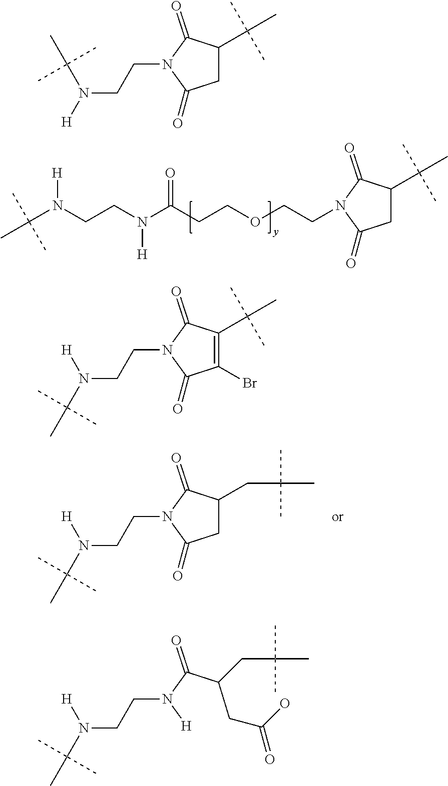

wherein y is an integer of 0 to 8
Ab is an antibody or an antigen binding fragment thereof, and
m, n, o and p represent integers of 0 or 1.

The linker (L) is preferably selected from

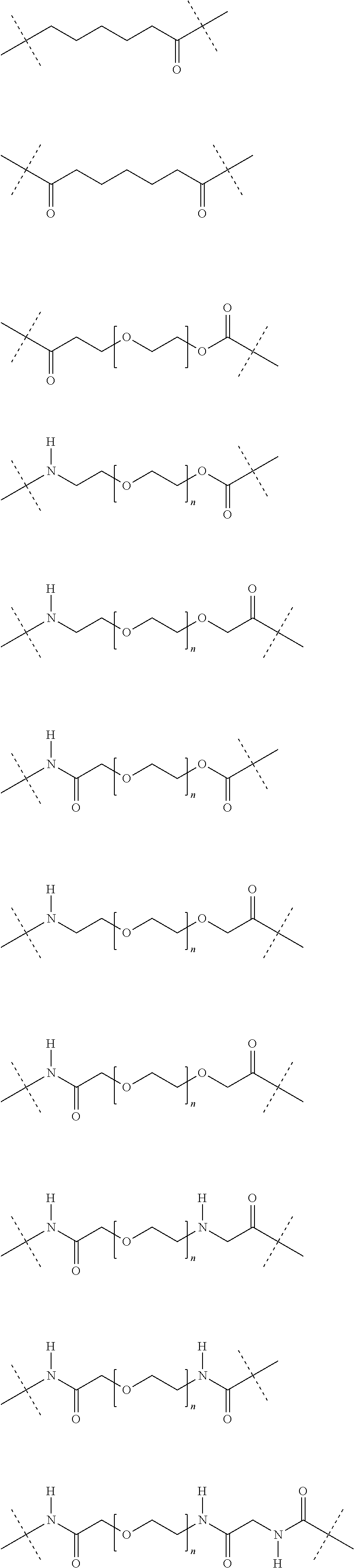

wherein n is an integer of 2 to 5

In particular, a payload is a toxin (HDAC inhibitor) linked to a suitable linker/spacer, which ends with groups (i.e., maleimide, NHS esters) suitable for conjugation to antibodies and comprises the following part of formula I:

D-(CU)ₘ-(S1)ₙ-L-(S2)ₒ-(CG)′ₚ Formula II

In the present invention, (CG) in the payloads of Formula II, may be NHS (N-Hydroxysuccinimide) or an activated acylderivative (including pentaflurophenyl ester, p-nitro and 2,4-dinitrophenol ester, thiophenol ester, acylimidazole, isobutylcarbonate, trichlorobenzoic anhydride), or maleimide- or 3-methylenesuccinimide, 3,4-dibromo maleimide or {amino-carbonyl}-3-butenoic acid of the following formulae:

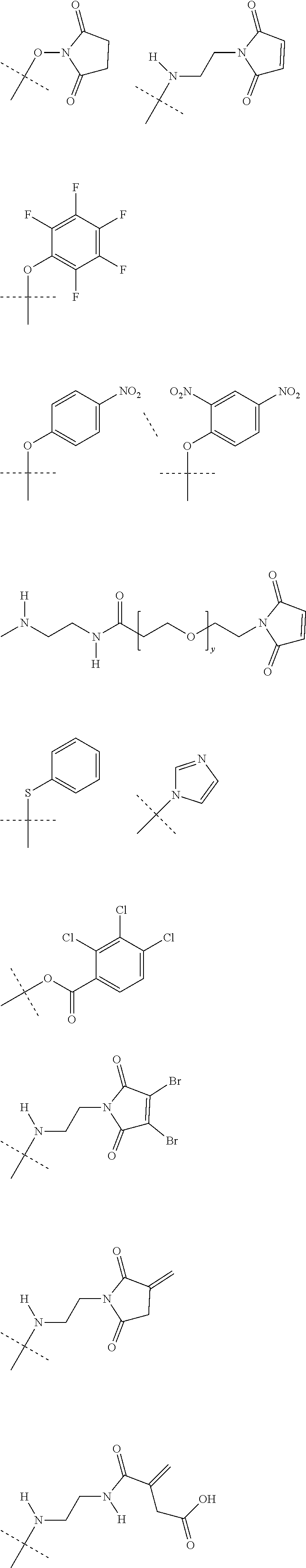

The histone deacetylase inhibitor (HDAC) used in the payload D-(CU)ₘ-(S1)ₙ-L-(S2)ₒ-(CG)ₚ- may be a histone deacetylase inhibitor known in the art, and be of the following categories:
Thiol-based histone deacetylase inhibitors, such as ST7464AA1 and ST7660AA1 (a thiol analogue of vorinostat), having the following formulas:

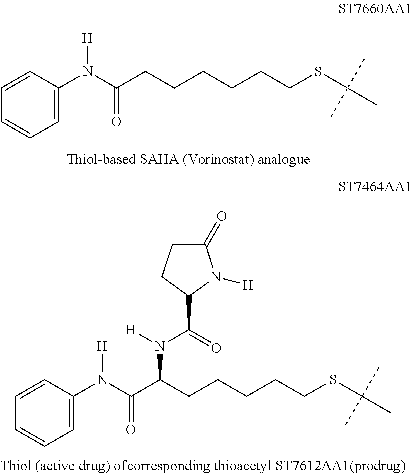

Hydroxamic acid based histone deacetylase inhibitors (HDACs), such as vorinostat (SAHA), panobinostat (LBH589), or dacinostat (LAQ824) having the following formulas:

| HDAC | ZBG (zinc binding group) | |
|---|---|---|
| Inhibitor | conjugation-based ADC | Other side conjugation-based ADC |
| Vorinostat (SAHA) | 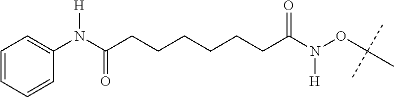 | |
| Panobinostat (LBH589) | 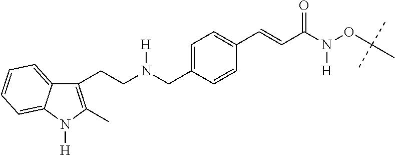 | 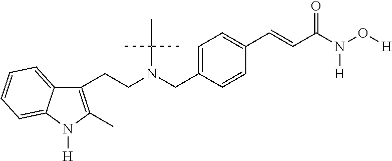 |
| Dacinostat (LAQ824) | 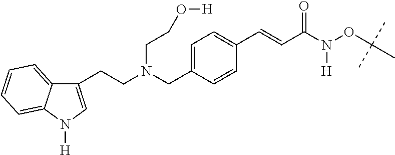 | 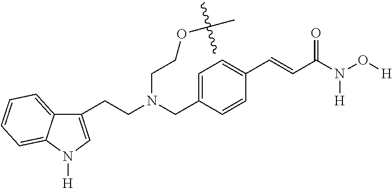 [SMILES: *OCCN(CCc1c[nH]c2ccccc12)Cc1ccc(/C=C/C(=O)NO)cc1] |

Benzamide based histone deacetylase inhibitors (HDACs) such as entinostat (MS275) and chidamide (CS055) having the following formula:

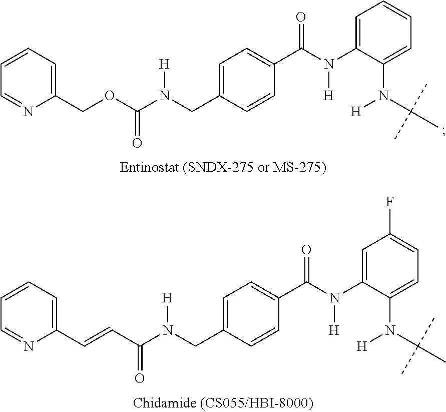

In a particular embodiment, the histone deacetylase inhibitor is ST7464AA1, drug of corresponding prodrug ST7612AA1, an oral thiol-based histone deacetylase inhibitor.

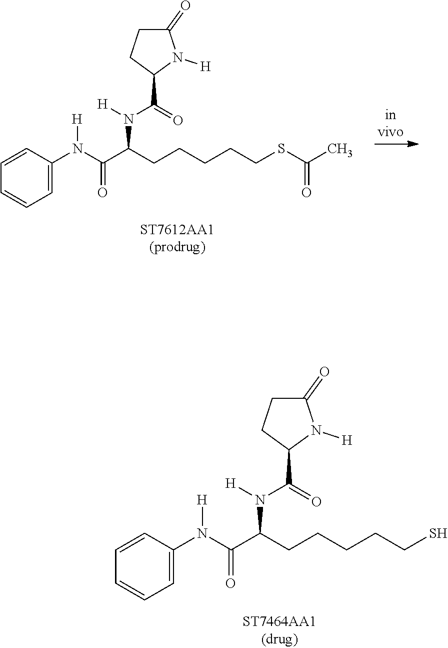 [SMILES: CC(=O)SCCCCC[C@H](NC(=O)[C@H]1CCC(=O)N1)C(=O)Nc1ccccc1 ; O=C1CC[C@H](C(=O)N[C@@H](CCCCCS)C(=O)Nc2ccccc2)N1]

The payload comprises a “leaving group”, which refers to a group that can be substituted by another group in a substitution reaction. Such leaving groups are well-known in the art and examples include, but are not limited to, halides (fluoride, chloride, bromide and iodide), azides, sulfonates (e.g., an optionally substituted C1-C6 alkanesulfonate, such as ethanesulfonate and trifluoromeethanesulfonate, or an optionally substituted C7-C12 alkylbenzenesulfonate, such as—toluenesulfonate), succinimide-N-oxide, p-nitrophenoxide, pentafluorophenoxide, tetrafluorophenoxide, arboxylates, aminocarboxylates (carbamates) and alkoxycarboxylates (carbonates). For substitutions at saturated carbon, halides and sulfonates are preferred leaving groups. For substitutions at a carbonyl carbon a halide, succinimide-N-oxide, p-nitrophenoxide, pentafluorophenoxide, tetrafluorophenoxide, a carboxylate, or an alkoxycarboxylate (carbonate) may for example be used as a leaving group. The term “leaving group” also refers to a group that is eliminated as a consequence of an elimination reaction, e.g., an electronic cascade reaction or a pirocyclization reaction. In this instance, a halide, a sulfonate, an azide, an aminocarboxylate (carbamate) or an alkoxycarboxylate (carbonate) may for example be used as a leaving group.

It is known to the person skilled in the art that transformation of a chemical functional group into another may require that one or more reactive centers in the compound containing such functional group have to be protected in order to avoid undesired side reactions. Protection of such reactive centres, and subsequent deprotection at the end of the synthetic transformations, can be accomplished following standard procedures described in the literature (see, for instance, Green, Theodora W. and Wuts, Peter G. M.—Protective Groups in Organic Synthesis, Third Edition, John Wiley & Sons Inc., New York (N.Y.), 1999).

TABLE 1
Payloads

| I.D. | ST code | Chemical Structure | M.W. | Class |
|---|---|---|---|---|
| (1) | 8128AA1 | 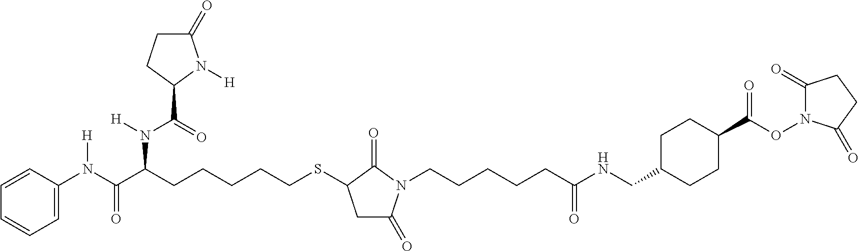 | 810.9 | NHS |
| (2) | 8152AA1 | 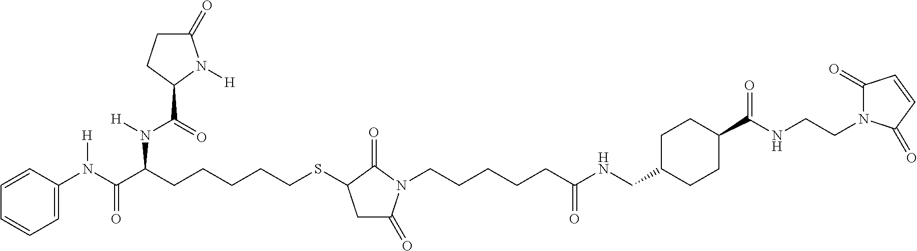 | 836.0 | Malei- mide |
| (3) | 8132AA1 | 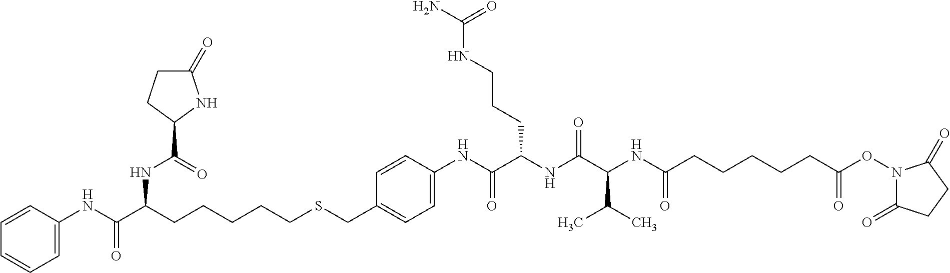 [SMILES: CC(C)[C@H](NC(=O)CCCCCC(=O)ON1C(=O)CCC1=O)C(=O)N[C@@H](CCCNC(N)=O)C(=O)Nc1ccc(CSCCCCC[C@H](NC(=O)[C@H]2CCC(=O)N2)C(=O)Nc2ccccc2)cc1] | 964.1 | NHS |
| (4) | 8190AA1 | 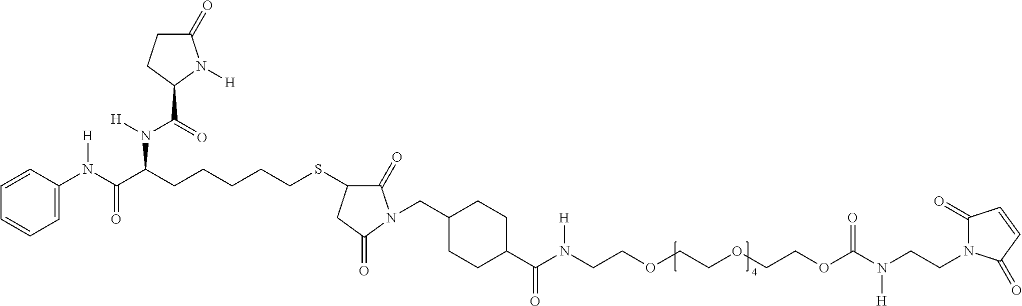 | 1030.2 | Malei- mide |
| (5) | 8189AA1 | 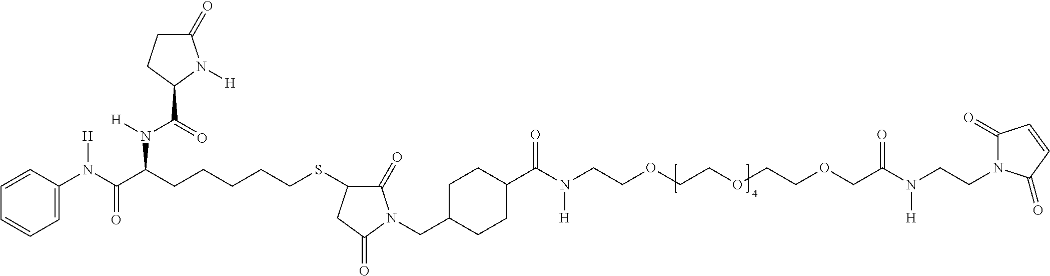 | 1044.2 | Malei- mide |
| (6) | 8191AA1 | 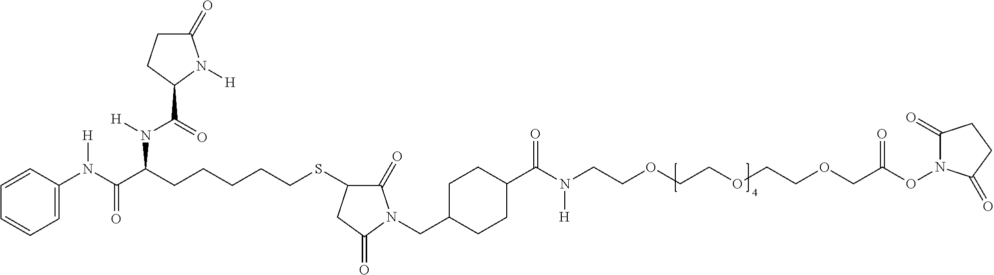 | 889.0 | NHS |
| (7) | 8197AA1 | 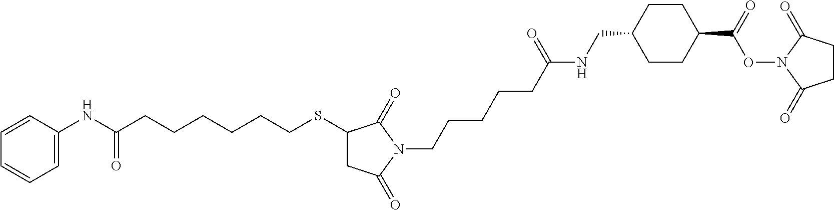 [SMILES: O=C(CCCCCN1C(=O)CC(SCCCCCCC(=O)Nc2ccccc2)C1=O)NC[C@H]1CC[C@H](C(=O)ON2C(=O)CCC2=O)CC1] | 684.8 | Malei- mide |
| (8) | 8235AA1 | 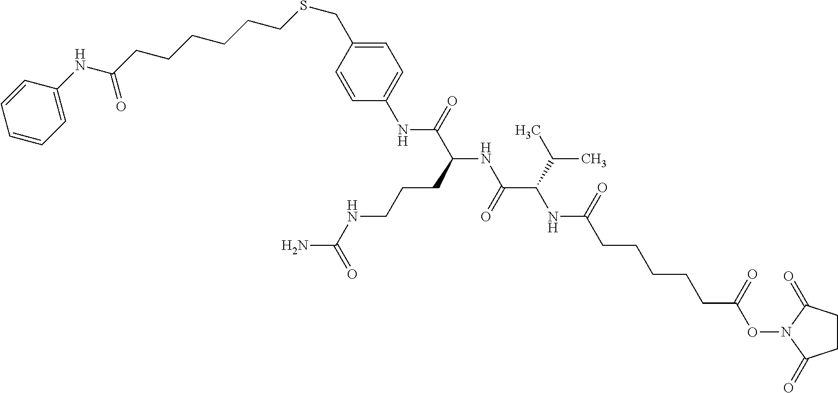 [SMILES: CC(C)[C@H](NC(=O)CCCCCC(=O)ON1C(=O)CCC1=O)C(=O)N[C@@H](CCCNC(N)=O)C(=O)Nc1ccc(CSCCCCCCC(=O)Nc2ccccc2)cc1] | 838.0 | NHS |
| (9) | 8217AA1 | 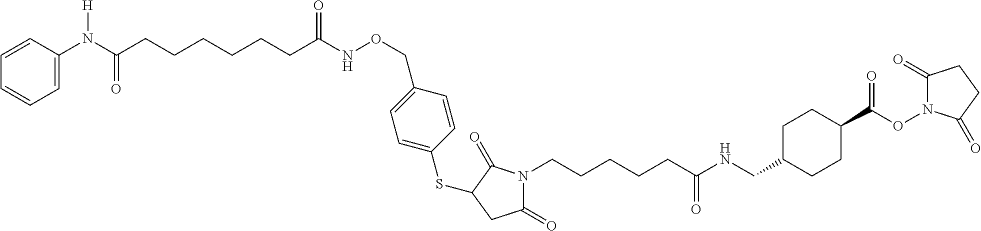 | 834.0 | NHS |
| (10) | 8201AA1 | 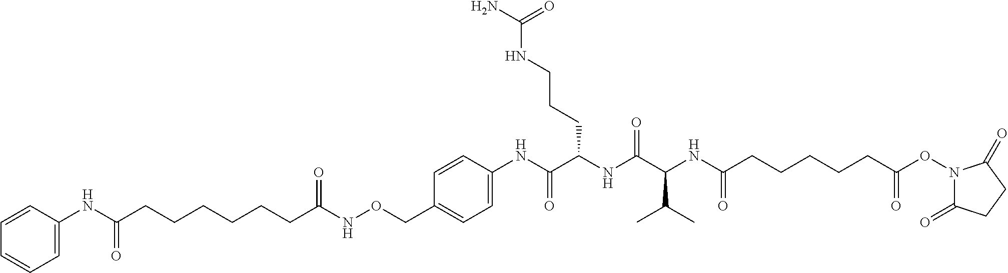 [SMILES: CC(C)[C@H](NC(=O)CCCCCC(=O)ON1C(=O)CCC1=O)C(=O)N[C@@H](CCCNC(N)=O)C(=O)Nc1ccc(CONC(=O)CCCCCCC(=O)Nc2ccccc2)cc1] | 865.0 | NHS |
| (11) | 8215AA1 | 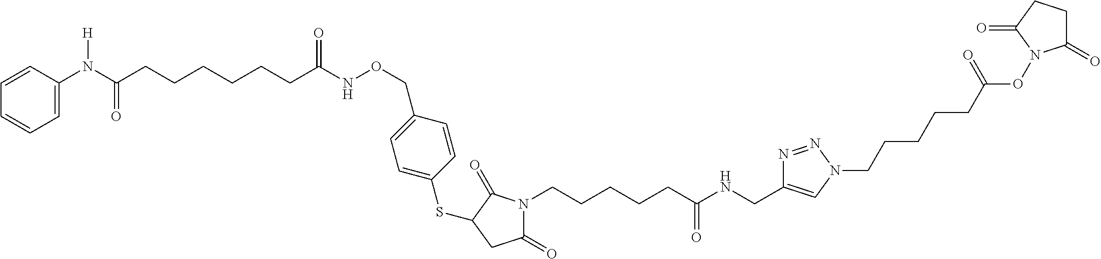 [SMILES: O=C(CCCCCN1C(=O)CC(Sc2ccc(CONC(=O)CCCCCCC(=O)Nc3ccccc3)cc2)C1=O)NCc1cn(CCCCCC(=O)ON2C(=O)CCC2=O)nn1] | 889.0 | NHS |
| (12) | 8216AA1 | 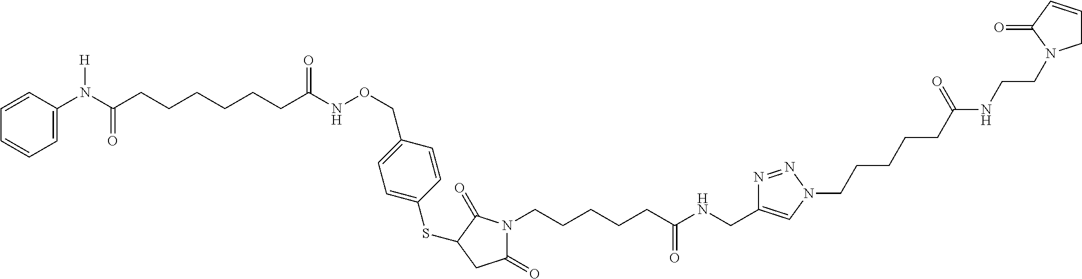 [SMILES: O=C(CCCCCn1cc(CNC(=O)CCCCCN2C(=O)CC(Sc3ccc(CONC(=O)CCCCCCC(=O)Nc4ccccc4)cc3)C2=O)nn1)NCCN1CC=CC1=O] | 914.1 | Malei- mide |
| (13) | 8236AA1 | 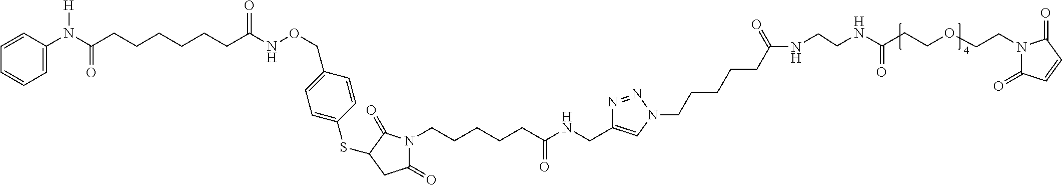 | 1161.4 | Malei- mide |
| (14) | 8232AA1 | 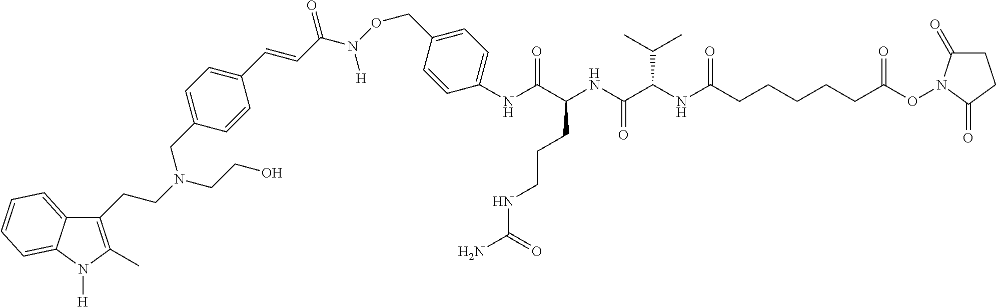 [SMILES: Cc1[nH]c2ccccc2c1CCN(CCO)Cc1ccc(/C=C/C(=O)NOCc2ccc(NC(=O)[C@H](CCCNC(N)=O)NC(=O)[C@@H](NC(=O)CCCCCC(=O)ON3C(=O)CCC3=O)C(C)C)cc2)cc1] | 994.1 | Malei- mide |
| (15) | 8233AA1 | 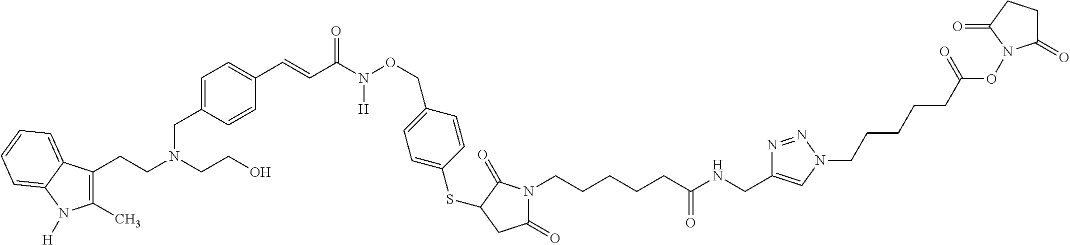 [SMILES: Cc1[nH]c2ccccc2c1CCN(CCO)Cc1ccc(/C=C/C(=O)NOCc2ccc(SC3CC(=O)N(CCCCCC(=O)NCc4cn(CCCCCC(=O)ON5C(=O)CCC5=O)nn4)C3=O)cc2)cc1] | 1018.2 | NHS |
| (16) | 8234AA1 | 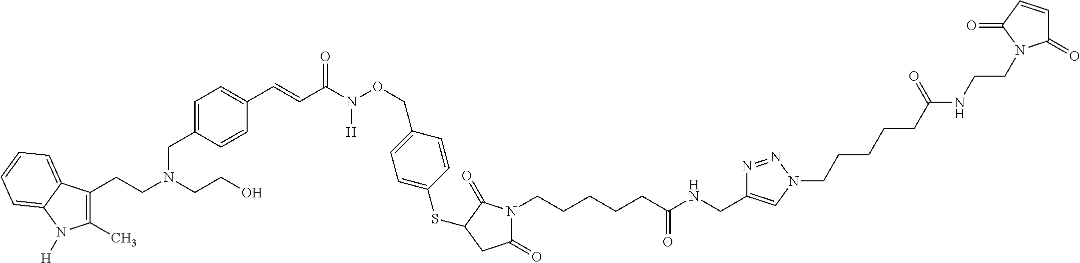 [SMILES: Cc1[nH]c2ccccc2c1CCN(CCO)Cc1ccc(/C=C/C(=O)NOCc2ccc(SC3CC(=O)N(CCCCCC(=O)NCc4cn(CCCCCC(=O)NCCN5C(=O)C=CC5=O)nn4)C3=O)cc2)cc1] | 1043.2 | Malei- mide |
| (17) | 8229AA1 | 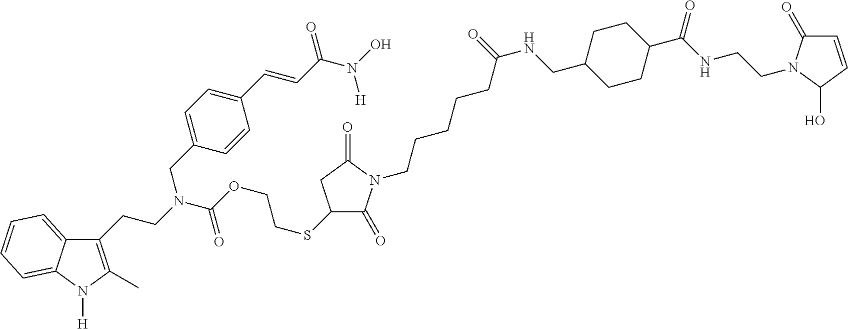 [SMILES: Cc1[nH]c2ccccc2c1CCN(Cc1ccc(/C=C/C(=O)NO)cc1)C(=O)OCCSC1CC(=O)N(CCCCCC(=O)NCC2CCC(C(=O)NCCN3C(=O)C=CC3O)CC2)C1=O] | 928.1 | Malei- mide |
| (18) | 8230AA1 | 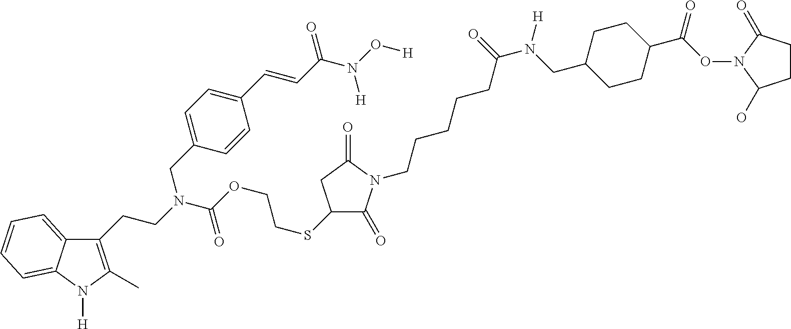 [SMILES: Cc1[nH]c2ccccc2c1CCN(Cc1ccc(/C=C/C(=O)NO)cc1)C(=O)OCCSC1CC(=O)N(CCCCCC(=O)NCC2CCC(C(=O)ON3C(=O)CCC3O)CC2)C1=O] | 903.0 | NHS |
| (19) | 8231AA1 | 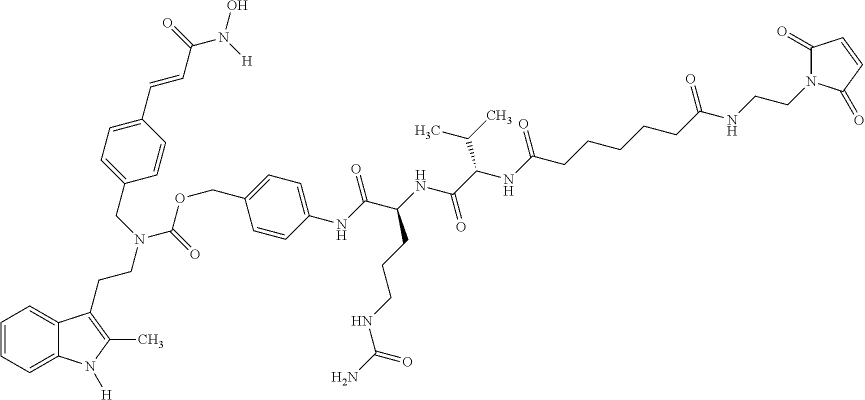 [SMILES: Cc1[nH]c2ccccc2c1CCN(Cc1ccc(/C=C/C(=O)NO)cc1)C(=O)OCc1ccc(NC(=O)[C@H](CCCNC(N)=O)NC(=O)[C@@H](NC(=O)CCCCCC(=O)NCCN2C(=O)C=CC2=O)C(C)C)cc1] | 1019.2 | Malei- mide |
| (20) | 8225AA1 | 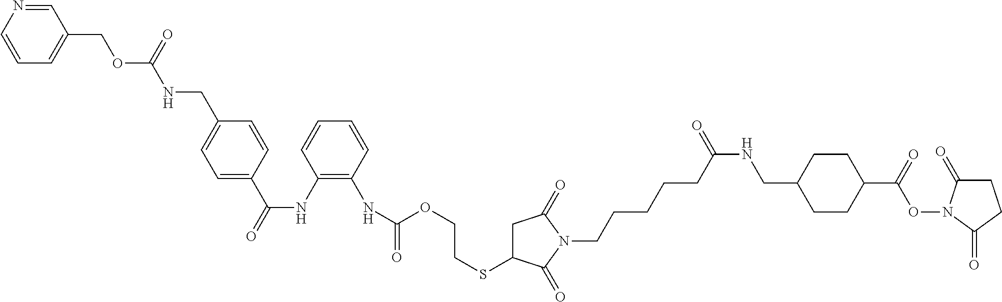 [SMILES: O=C(CCCCCN1C(=O)CC(SCCOC(=O)Nc2ccccc2NC(=O)c2ccc(CNC(=O)OCc3cccnc3)cc2)C1=O)NCC1CCC(C(=O)ON2C(=O)CCC2=O)CC1] | 928.0 | NHS |
| (21) | 8226AA1 | 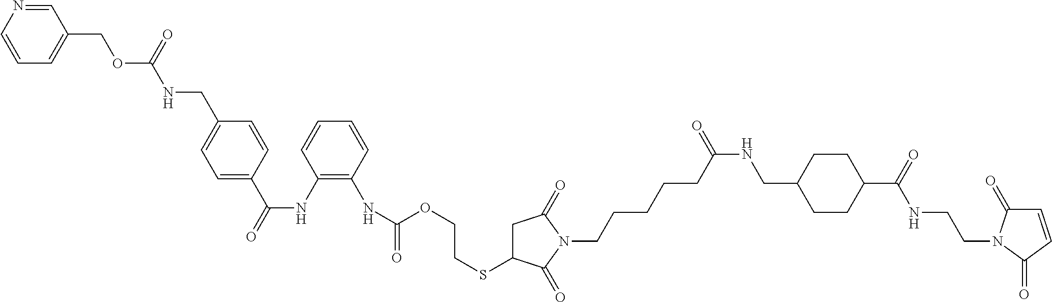 [SMILES: O=C(CCCCCN1C(=O)CC(SCCOC(=O)Nc2ccccc2NC(=O)c2ccc(CNC(=O)OCc3cccnc3)cc2)C1=O)NCC1CCC(C(=O)NCCN2C(=O)C=CC2=O)CC1] | 953.1 | Malei- mide |
| (22) | 8227AA1 | 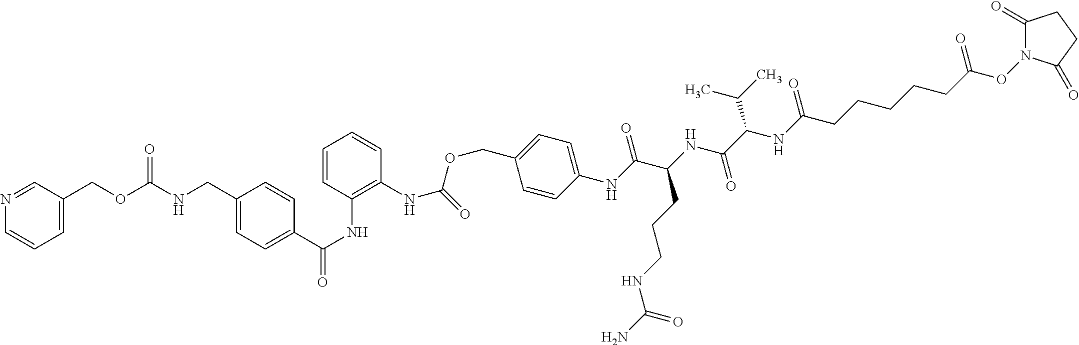 [SMILES: CC(C)[C@H](NC(=O)CCCCCC(=O)ON1C(=O)CCC1=O)C(=O)N[C@@H](CCCNC(N)=O)C(=O)Nc1ccc(COC(=O)Nc2ccccc2NC(=O)c2ccc(CNC(=O)OCc3cccnc3)cc2)cc1] | 1021.1 | NHS |
| (23) | 8228AA1 | 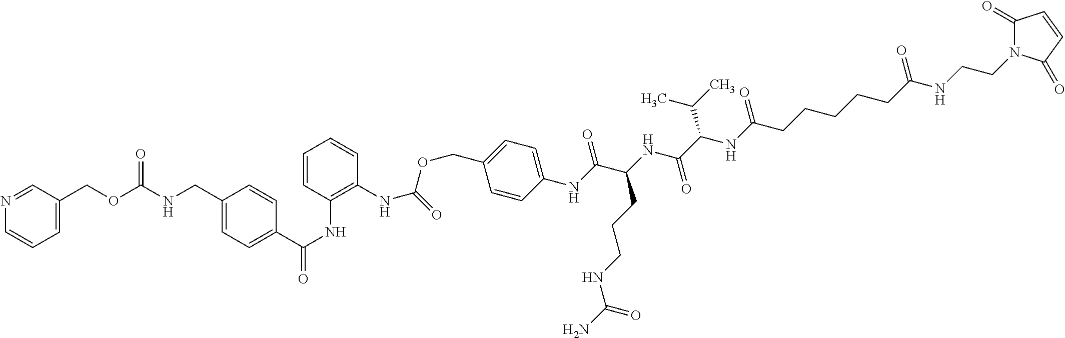 [SMILES: CC(C)[C@H](NC(=O)CCCCCC(=O)NCCN1C(=O)C=CC1=O)C(=O)N[C@@H](CCCNC(N)=O)C(=O)Nc1ccc(COC(=O)Nc2ccccc2NC(=O)c2ccc(CNC(=O)OCc3cccnc3)cc2)cc1] | 1046.1 | Malei- mide |

In a particular preferred embodiment, the histone deacetylase inhibitor compound ST7612AA1 being a prodrug of the compound ST7464AA1 as show above.

Further examples of payloads useful in the present invention are the compounds of the above Table 1. Payloads with a specific linker containing N-hydroxysuccinimide (NHS) moiety (i.e. ST8128AA1, ST8132AA1) are able to covalently bound, through an amide bond, to the side chain of a Lys residue of mAbs that contains an amino-specific NHS ester may that reacts with antibody lysines. Payloads with a specific linker containing maleimide moiety (i.e. ST8152AA1, ST8189AA1) are able to covalently bound, through a maleimide-thiol conjugation reaction to Cys on mAbs, after their reduction.

The antibody-drug-conjugates (ADCs) differ from the payloads for the connective group (CG) where, in the payloads (CG)′ it was NHS or Maleimide while in the antibody conjugates, (CG) is absent, in the NHS payload-based ADCs, while it is a succinimidyl moiety in Maleimide payload-based ADCs.

Moreover, the spacer (S1), if it is present, may be cleavable or non-cleavable. A typical protease-cleavable spacer contain a moiety characterize by a fast enzymatic release of the drug in the target cell, such as the valine-citrulline (Val-Cit) dipeptide.

An example of a cleavable spacer is

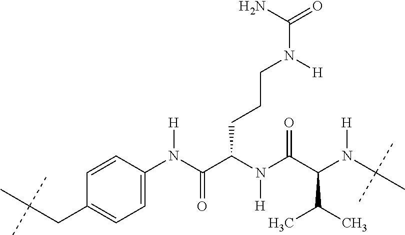

Examples of a non-cleavable spacer are

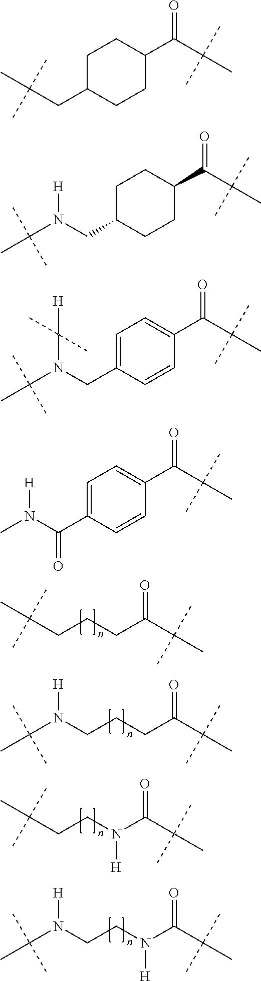

n=0-4

The claimed antibody drug conjugates may further contain a linker (L), which may be (CH₂)q-CO, NH—(CH₂)r-(PEG)s-(CH₂)w-CO, NH—CO—(CH₂)r-(PEG)s-X—(CH₂)w-CO, where X may be absent, NH, O, q is 2-8, r is absent or 1-4, s is absent or 1-6, and w is absent or 1-2

Examples of linker (L):

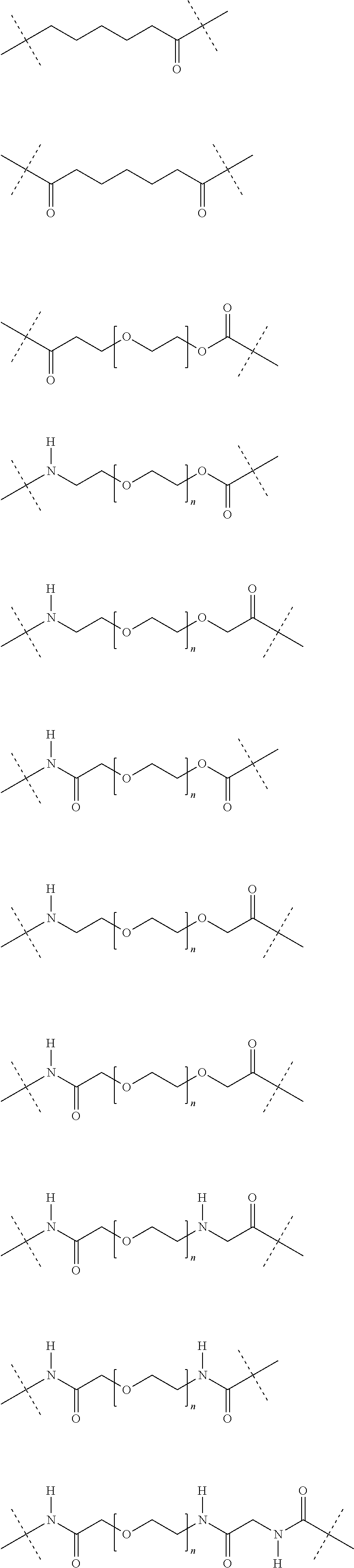

n=2-5

The claimed antibody-drug-conjugates may further contain a connecting group (CG) formed after conjugation to the cysteine thiol- or lysine amino-groups of the antibodies, which can be absent or one of following moiety: NHS or an activated acylderivative (including 1-hydroxybenzotriazole ester, ethyl 2-cyano-2-(hydroximino) acetate ester, N-ethoxycarbonyl-2-ethoxy-1,2-dihydro-quinolone ester, pentaflurophenyl ester, p-nitro and 2,4-dinitrophenol ester, thiophenol ester, acylimidazole, isobutylcarbonate, trichlorobenzoic anhydride, pivalic anhydride, 3,5-dimethoxytriazine) or maleimide- or 3-methylenesuccinimide, 3,4-dibromo maleimide or {amino-carbonyl}-3-butenoic acid of the following formulae:

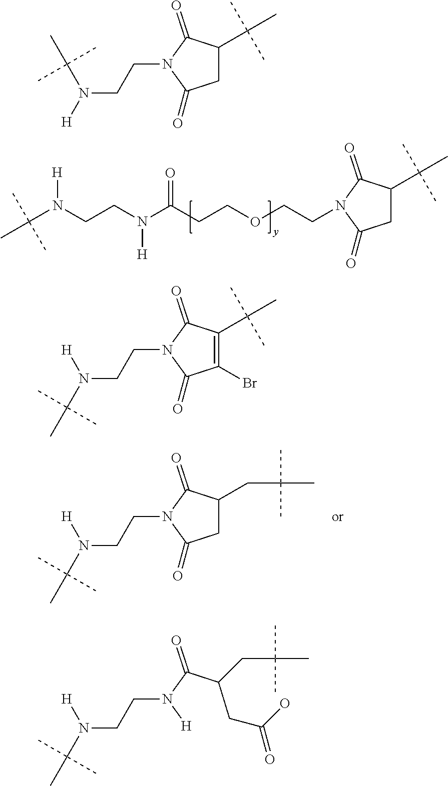

where y is 0-8

The immunoglobulin vectors herein described are directed against receptors of the tyrosine kinase (RTK) family. This is a superfamily of transmembrane proteins that mediate intracellular signaling by phosphorylating substrate proteins involved in cell proliferation, survival, differentiation or migration.

In particular, the Human Epidermal growth factor Receptor (HER) family belongs to the RTKs superfamily, and comprises four members: ErbB1/EGFR (epidermal growth factor receptor), ErbB2, ErbB3 and ErbB4. Physiologically, these receptors are activated by the ligands of the EGF family.

EGFR plays a causal role in the development and maintenance of many human carcinomas, with mutation and overexpression observed in a number of tumor types (Burgess A W 2008 Growth Factors 26: 263-74).

EGFR has become a clinically validated target for antibodies as well as for tyrosine kinase inhibitors having gained widespread use in lung, head and neck, colon, and pancreatic cancers (Mendelsohn J 2006 Semin Oncol 33: 369-85; Feiner 2016 Exp Rev Proteomics, September 13: 817-32; Enrique AA 2012 Front Biosci 4: 12-22; Landi L 2014 Expert Opin Pharmacol Ther 15: 2293-305).

Despite the success of these inhibitors, significant numbers of patients with EGFR-positive tumors fail to respond to current EGFR-targeting therapeutics as a range of mutations (e.g., EGFR, KRAS, BRAF, PI3K, and PTEN) may contribute to intrinsic or acquired resistance (Chong C R 2013 Nat Med 19: 1389-400).

A microtubule inhibitor-based ADC targeting EGFR is a questionable therapeutic strategy in that it may improve the activity of anti-EGFR antibodies by circumventing resistance mediated by downstream signaling mutations, but, because of the known toxicity of these antibodies (i.e. skin rash, diarrhea, constipation, stomatitis, fatigue, and electrolyte disturbances) (Li T 2009 Target Oncol 4: 107-19) it might have limited applicability. It was surprisingly found that ADCs made of anti-EGFR family protein antibodies conjugated to low toxicity HDACi are effective anti-cancer agents.

The antibody used in the claimed antibody-drug-conjugate is particularly an antibody directed against an EGFR family protein. Specifically, the antibody may be directed to the ErbB1, ErbB2 or ErbB3 receptors. The same payloads conjugated to other antibodies can be directed against other receptors internalized by tumor cells to release the HDACi.

For example, in analogy to EGFR, c-Met implicated in the growth, survival and spread of various human cancers and overexpressed in different solid tumors is internalized in response to HGF (hepatocyte growth factor) binding, leading to c-Met ubiquitination and degradation (Mellman 2013 Cold Spring Harb Perspect Biol 5:a016949).

In addition, integrins have major roles in tumor-stroma interactions and aberrant recycling of 25 different integrin heterodimers is involved in tumor growth, invasion, metastasis and evasion of apoptosis (Mosesson 2008 Nat Rev Cancer 8: 835-50).

In one embodiment, the antibody is selected from Trastuzumab, Cetuximab, Bevacizumab, Panitumumab, anti-CD4, or anti-CD30 antibodies, and related bio-similar antibodies.

The terms antibody or immunoglobulin may be used interchangeably in the broad-er sense and include monoclonal antibodies, polyclonal antibodies, isolated, engineered or recombinant antibodies, full-length or intact antibodies, multivalent or multispecific antibodies such as bispecific antibodies or antibody fragments thereof as long as they exhibit the desired biological activity. In case of a recombinant antibody, which is the result of the expression of recombinant DNA within living cells, the antibody may be derived from any species and is preferably derived from humans, rats, mice and rabbits. If the antibody is derived from a species other than a human species, it is preferably a chimeric or humanized antibody prepared according to techniques well-known in the art.

The antibody may also be a chemically synthesized antibody.

The antibody can target the cancer or tumour cells in question, in particular cancer or tumor cells expressing ErbB1, ErbB2 and/or ErbB3 receptors. Particularly, the antibody has the property of recognizing said cancer or tumor cells, has a property of binding to said cancer or tumor cells and a property of internalizing into a tumor or a cancer cell.

Methods of preparing said antibodies are well-known in the art [i.e., Chem. Soc. Rev., 2016, 45, 1691-1719; Bioorganic & Medicinal Chemistry Letters 26 (2016) 1542-1545].

Instead of an antibody, an antigen-binding fragment can also be used which indicates any peptide, polypeptide or protein retaining the ability to bind to the target (antigen) of the antibody. Examples of antigen-binding fragments are Fv, ScFv (Sc means single-chain), Fab, F(ab′)2, Fab′, ScFv′, Fc fragments or Diabodies or fragments the half-life of which has been increased by a chemical modification, such as, for example, pegylation or by incorporation into a liposome.

In an embodiment of antibody drug conjugate (ADC), or pharmaceutically acceptable salt thereof are selected by the compounds having the formulae:

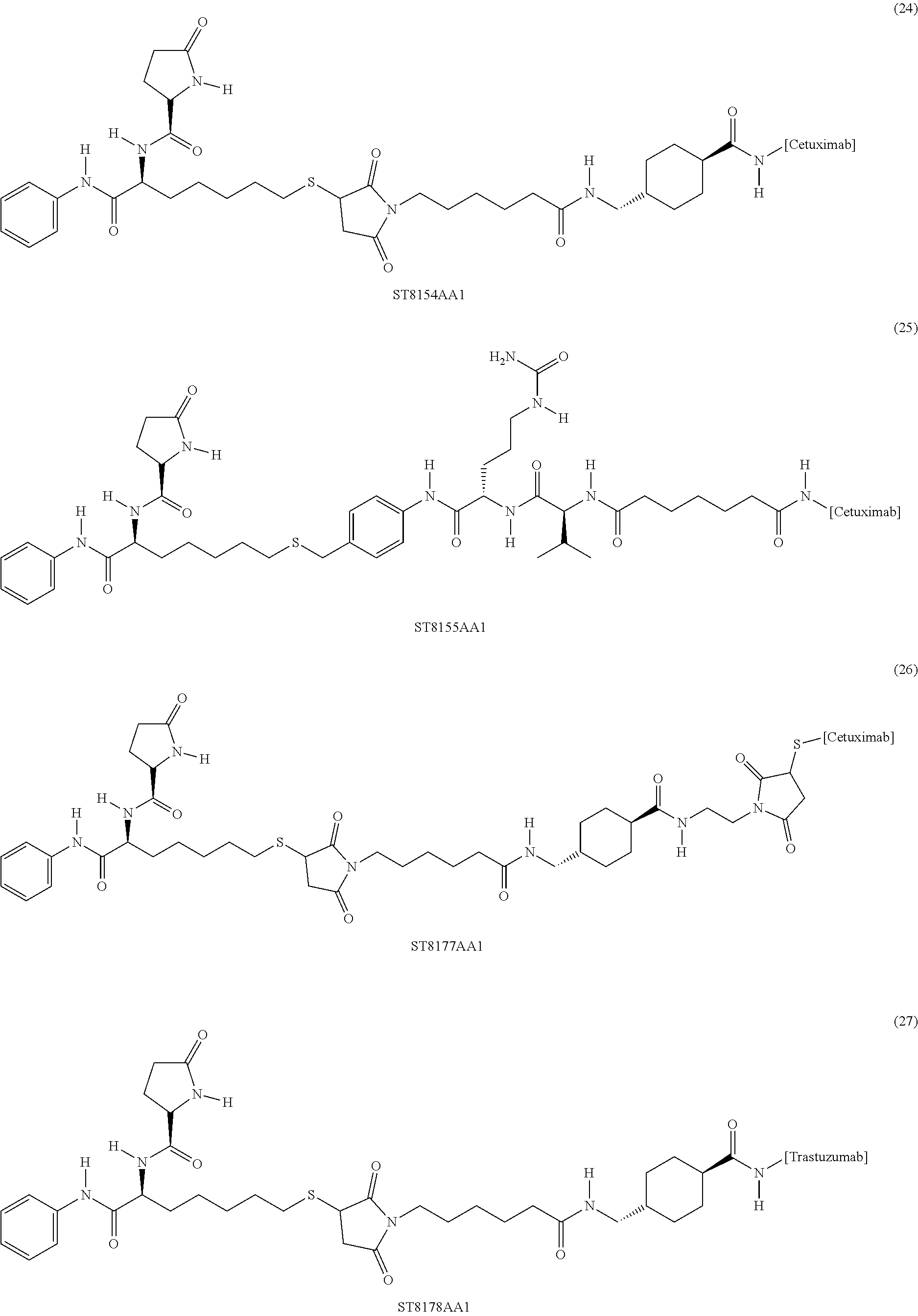

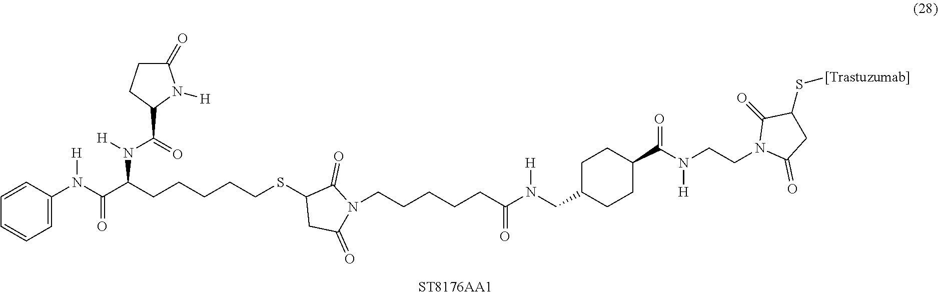

In another embodiment of Formula I, there is provide a compound, such as an antibody drug conjugate (ADC), or pharmaceutically acceptable salt thereof, of Formula (If):

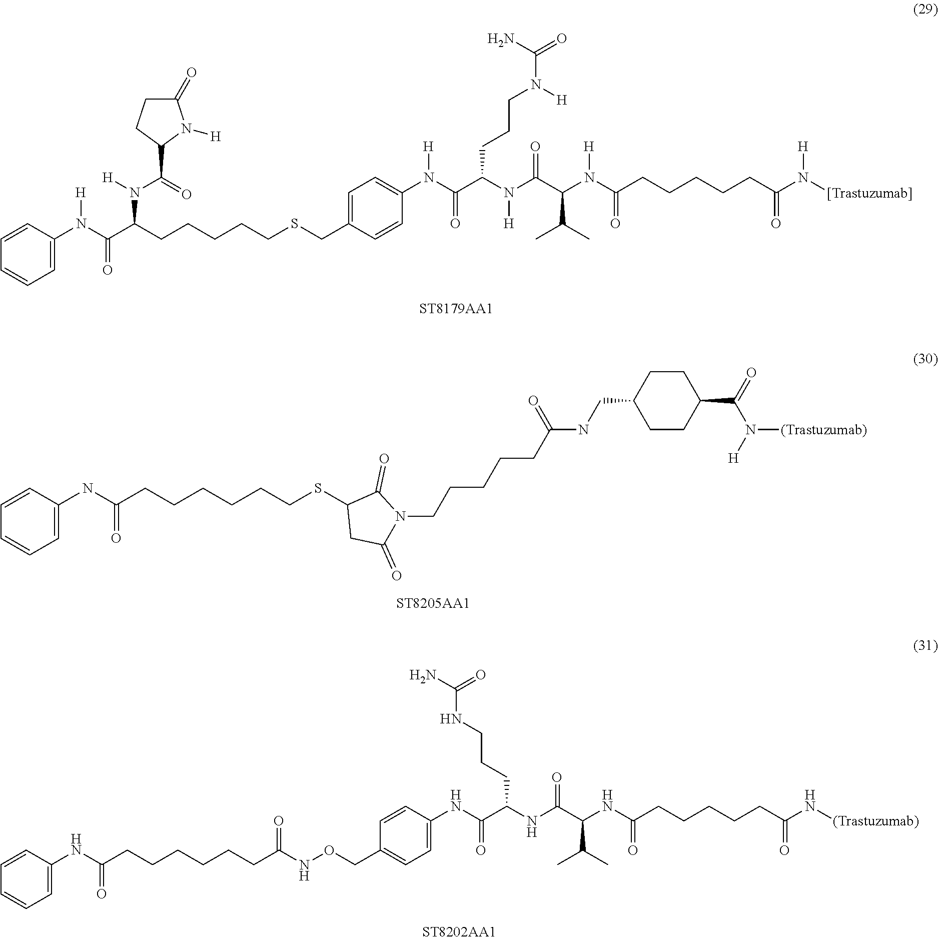

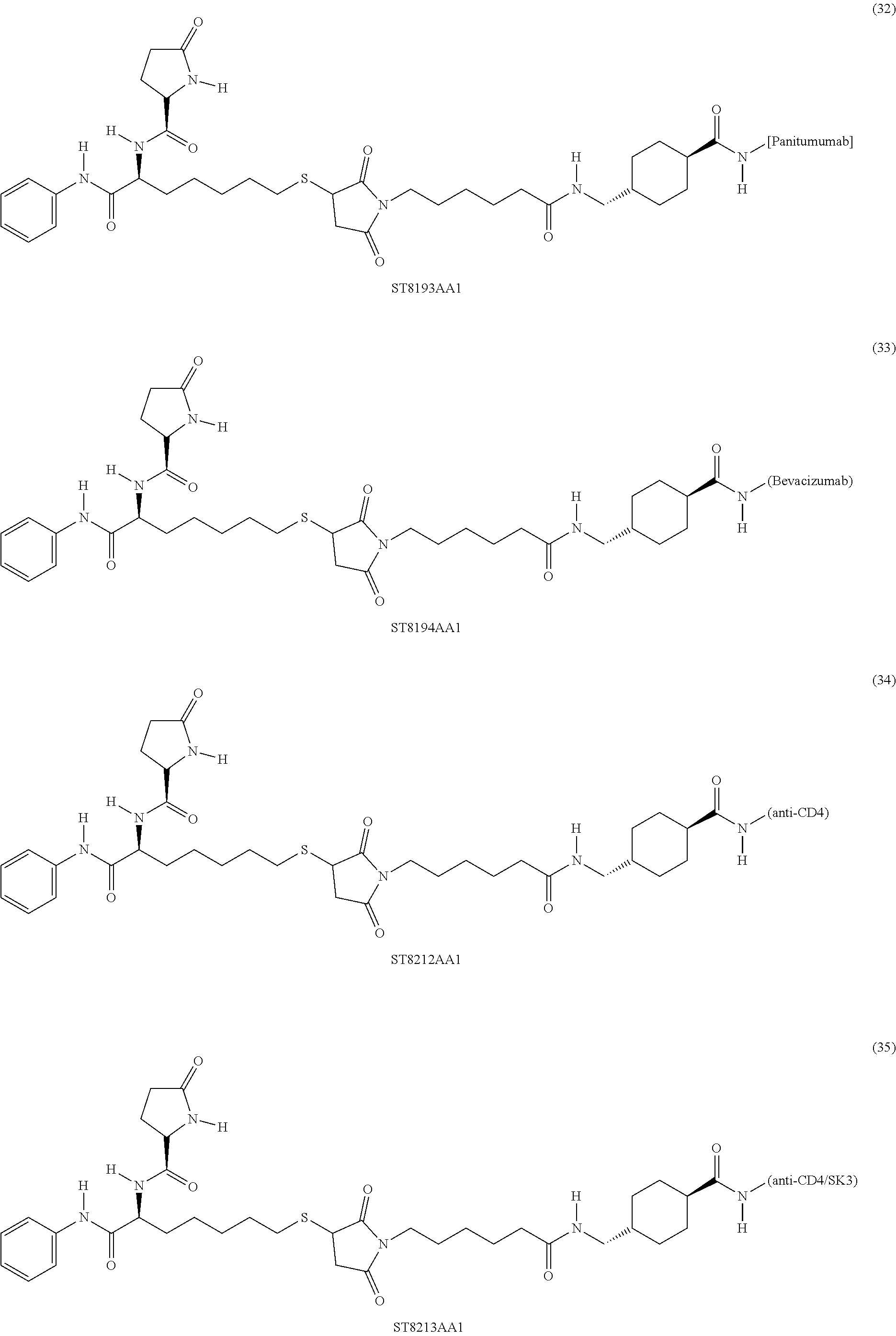

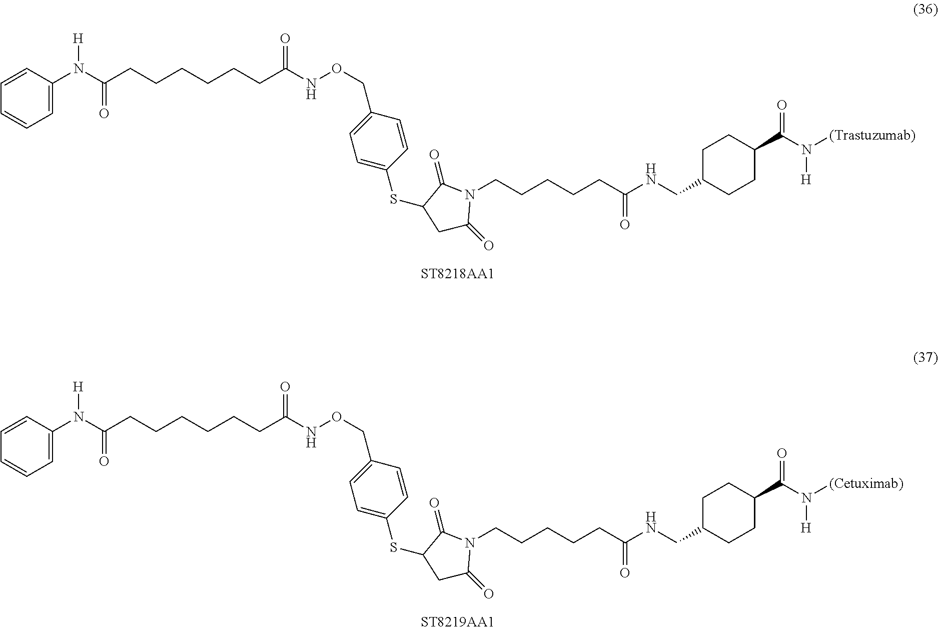

In the following a general scheme of HDACi-based ADCs is shown. The active drug (D) is represented by ST7464AA1 that corresponds to the active drug of ST7612AA1. ST7464AA1 is one of the possible drugs (D) of ADCs (Formula I) and of payloads (Formula II), described in the present invention.

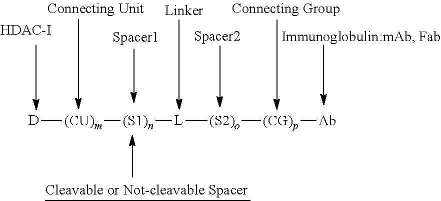

The above is a general scheme of representative HDAC inhibitors-based Antibody Drug Conjugates (ADCs)

In another embodiment is the process for the preparation of payload, which comprises the introduction on the thiol, hydroxamic or benzamide residues of the HDAC inhibitor, of a suitable Connecting Unit (C.U.) to which to link the other components of the payload, if any. A spacer (S1)—that can be cleavable (normally the valine-citrulline dipeptide (Val-Cit), substrate of enzyme cathepsin), or not cleavable—a linker (L), a second spacer (S2) and a terminal group (CG)′, suitable for conjugation to lysine or cysteine antibody.

In another embodiment is the process for the preparation of HDAC inhibitors-based Antibody Drug Conjugates (ADCs), which normally consists of a conjugation reaction between the residue (CG)′ of the payload part of the molecule and an amino group of a lysine residue of the immunoglobulin (Ab). For conjugation to cysteines, the S—S cysteine bonds of the immunoglobulin have to preliminarily be reduced with an appropriate reducing agent. On the sulfhydryl groups (—SH) of free cysteine residues, proceed with the subsequent reaction of conjugation with a payload having an appropriate group (CG)′.

TABLE 2
ADCs

| I.D. ST code | Chemical Structure | SoC | DAR |
|---|---|---|---|
| (24) 8154AA1 | 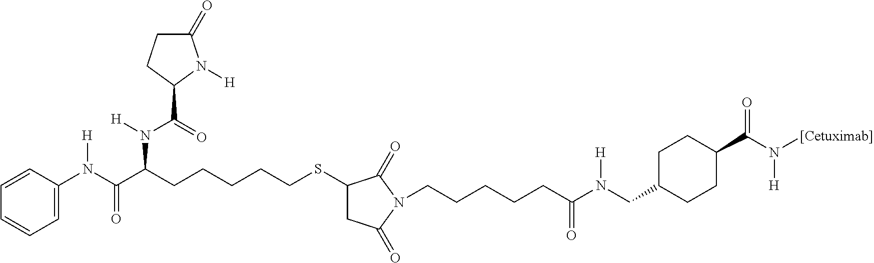 | Lys | 8.0 (±1) |
| (25) 8155AA1 | 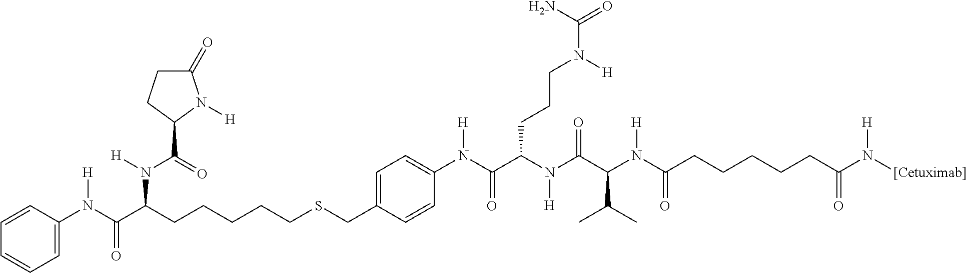 | Lys | 6.1 (±1) |
| (26) 8177AA1 | 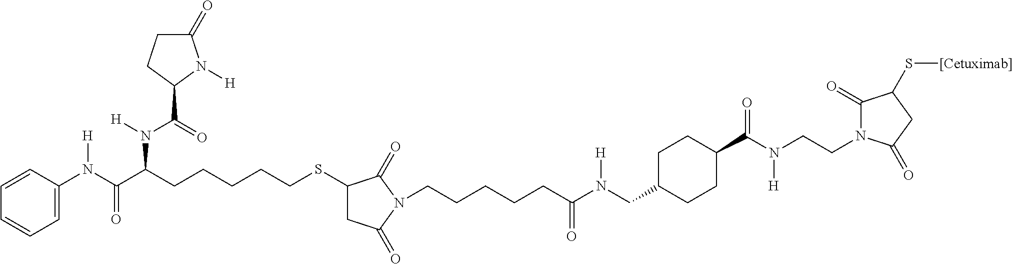 | Cys | 4.0 (±0.7) |
| (27) 8178AA1 | 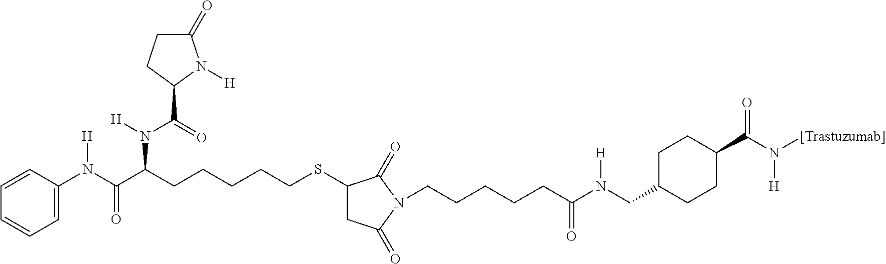 | Lys | 8.0 (±0.2) |
| (28) 8176AA1 | 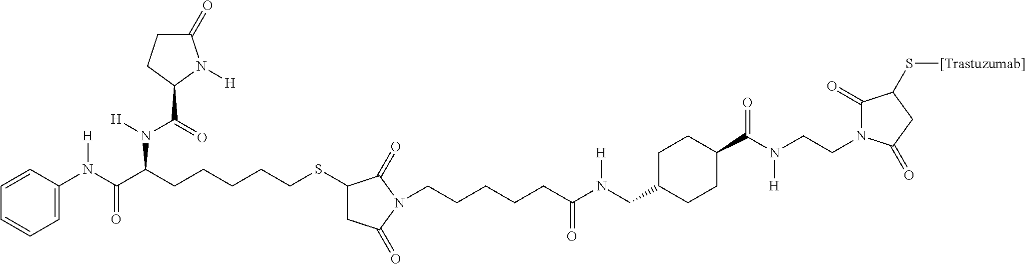 | Cys | 4.5 (±0.5) |
| (29) 8179AA1 | 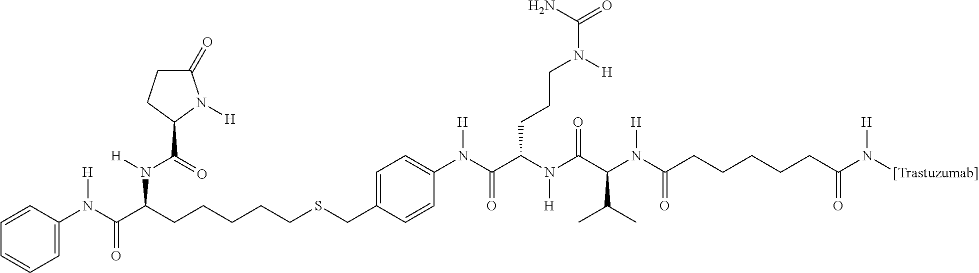 | Lys | 6.1 (±0.2) |
| (30) 8205AA1 | 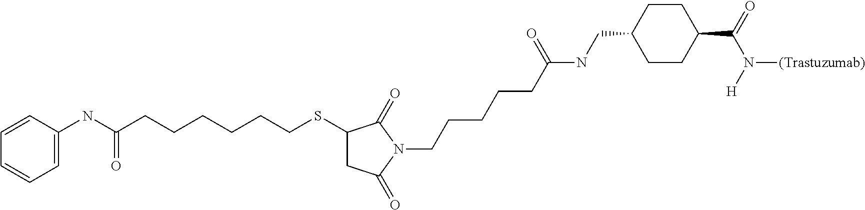 | Lys | 6.2 (±0.2) |
| (31) 8202AA1 | 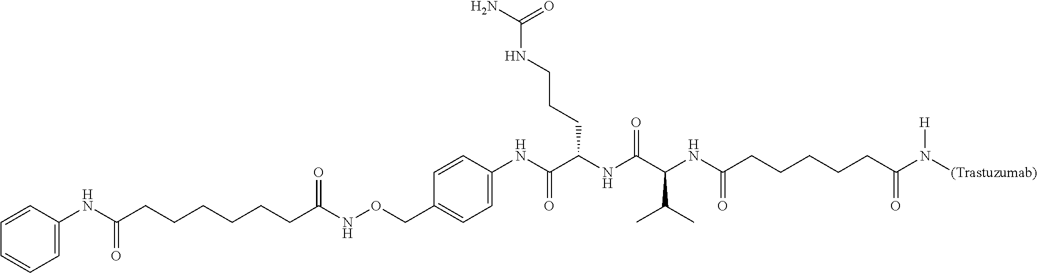 | Lys | 5.5 (±0.5) |
| (32) 8193AA1 | 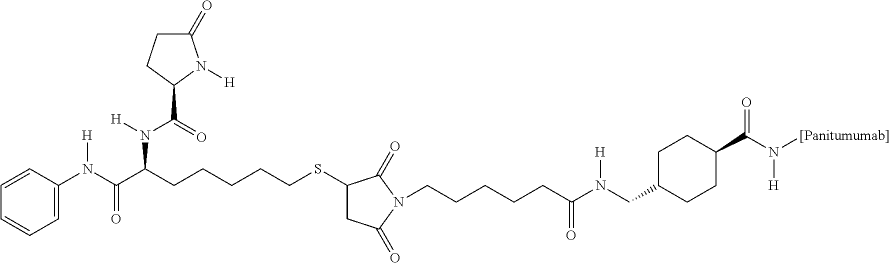 | Lys | 9.0 (±2) |
| (33) 8194AA1 | 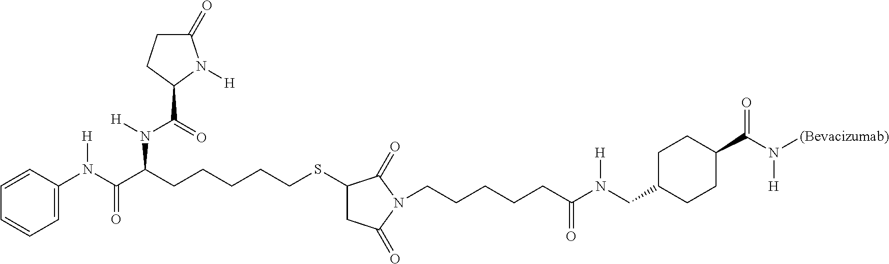 | Lys | 6.5 (±0.5) |
| (34) 8212AA1 | 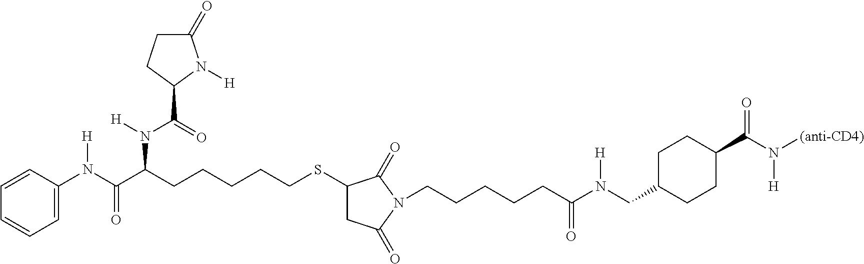 | Lys | 8.0 (±0.2) |
| (35) 8213AA1 | 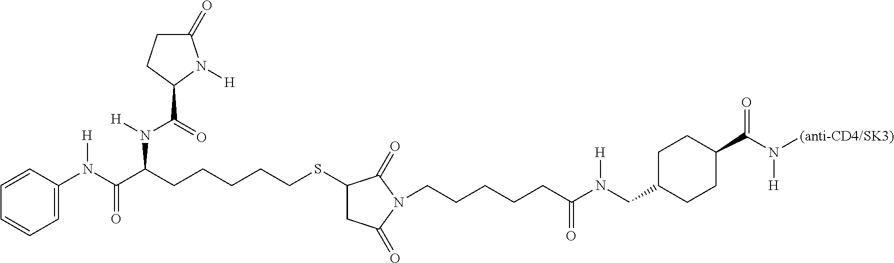 | Lys | 4.0 (±1) |
| (36) 8218AA1 | 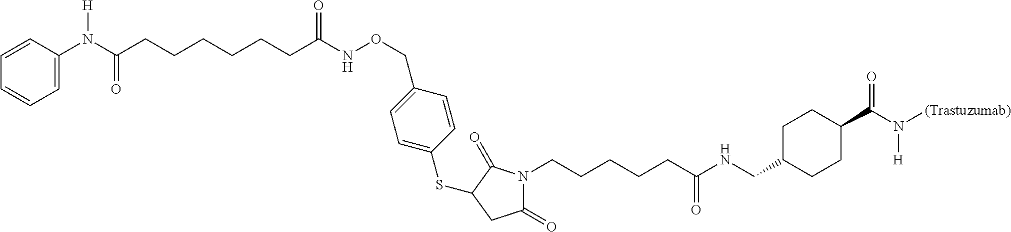 | Lys | 5.0 (±0.5) |
| (37) 8219AA1 | 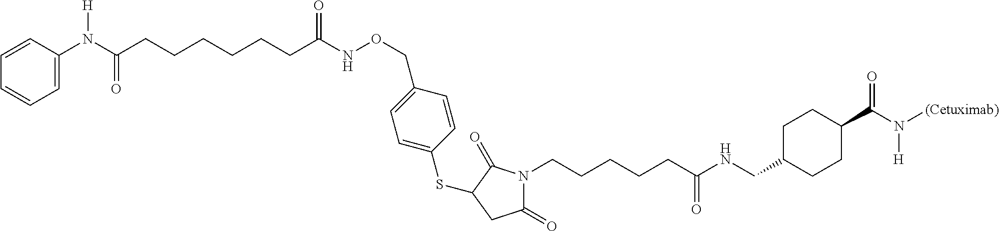 | Lys | 4.0 (±0.5) |

SoC: Site of Conjugation

The present invention also relates to a pharmaceutical composition comprising the above antibody-drug conjugates. Said pharmaceutical compositions contain at least an excipient and/or a pharmaceutically acceptable vehicle. The active ingredient can be administered in unit form of administration in admixture with conventional pharmaceutical carriers to animals or to human beings. Suitable unit forms of administration comprise forms for enteral or parenteral administration, wherein the enteral administration comprises oral, aerosol, rectal or buccal route, and parenteral administration comprises subcutaneous, intramuscular or intravenous, and intradermic route.

Pharmaceutical composition may be for enteral or parenteral administration, wherein the enteral administration comprises oral, aerosol, rectal or buccal route, and parenteral administration comprises endovenous, intramuscolar, and intradermic route.

A solid composition for oral administration can be a tablet, a pill, a powder, a capsule or a granulate. In these compositions the antibody-drug-conjugate of the invention is mixed with one or more inert diluent such as starch, cellulose, sucrose, lactose or silica. These compositions may also comprise further substances, such as lubricants, such as magnesium stearate or talc or coloring agents or coating agents.

A sterile composition for parenteral administration may preferably be an aqueous or non-aqueous solution, suspension or emulsion. A solvent or vehicle used can be made of water, propylene glycol or polyethylene glycol, vegetable oils, injectable organic esters or other suitable organic solvents. These compositions may also contain adjuvants, in particular wetting, isotonic, emulsifying, dispersing and stabilizing agents.

The present invention reports in vitro cell proliferation assay at demonstration of the efficacy provided by the new ADCs.

A particular embodiment of the invention is a formulation suitable for local delivery by nebulization. In this regard the antibody-drug-conjugates of the invention are particularly suitable in the treatment of diseases associated with the lung or the peritoneum such as lung or peritoneal cancer or cancer from ovarian, cervix-endometrium, gastric, colon, appendiceal, pseudomyxoma peritonei, pancreas, liver metastases, rare neoplasie (abdominal sarcoma of not gut tissues).

The present invention also provides a compound of formula (I) as defined above, for use in a method of treating cancer, cellular proliferation disorders and viral infections.

Preferably, a compound of formula (I) as defined above, is for use in a method of treating specific types of cancers, such as but not limited to: carcinomas, including bladder, breast, colon, kidney, liver, lung, comprising small cell lung cancer, esophagus, gall-bladder, ovary, pancreas, stomach, cervix, thyroid, prostate, and skin carcinoma, comprising squamous cell carcinoma; hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkitt's lymphoma; hematopoietic tumors of myeloid lineage, including acute and chronic myelogenous leukemia, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannoma; and other tumors, including melanoma, seminoma, teratocarcinoma, osteosarcoma, xeroderma pigmentosum, keratoxanthoma, thyroid follicular cancer, Kaposi's sarcoma and mesothelioma.

The present invention reports in vitro cell proliferation assay at demonstration of the efficacy provided by the new ADCs.

The compounds of the invention bind to the receptor in a comparable manner to that of the free antibodies and the binding of the ADCs to ErbB1- and ErbB2-expressing tumour cell was confirmed by FACS analysis.

Through fluorescence analysis, it has been demonstrated that the compounds of the invention are able to internalize with tumour cells in a comparable manner to their native counterpart antibodies without any reduction in the binding rate. Moreover, all the ADCs of the invention react with their specific receptor with potency comparable to that of a native antibodies bound to the ADC, as for example Cetuximab or Trastuzumab.

The ADCs of the invention maintain their integrity when nebulized and thereof they can be comprised in composition for nebulization, which represent a powerful method for deliver mAbs in respiratory diseases. This method is a non-invasive method suitable for targeting drugs to the lungs, limiting the exposure to secondary organs.

The compounds of the invention inhibit tumour cell proliferation with IC₅₀ values lower than those of cetuximab alone, when evaluated on lung adenocarcinoma cells, thus confirming their antitumor efficacy.

All tested ADCs induced a relevant increase in the acetylation level of both α-tubulin and histone H3 in all tested tumour cell lines, because of direct enzymatic inhibition of HDAC6 and class I HDACs, respectively.

In vivo experiment have demonstrated that the compounds of the invention are more efficient in inhibiting the growth of lung, colon, pancreas, ovarian tumours as compared to Cetuximab and Trastuzumab alone.

The new ADCs show an antitumor activity by aerosol delivery and intraperitoneal route on a local tumour, useful for pressurized intraperitoneal aerosol chemotherapy (PIPAC).

### EXAMPLES

The present invention will now be further described with reference to the following examples. It will be appreciated by a person skilled in the art that these examples are only for illustrative purpose and are not to be construed to limit the scope of the present invention.

### Example 1: Synthesis and Characterization of Payload (1), ST8128AA1

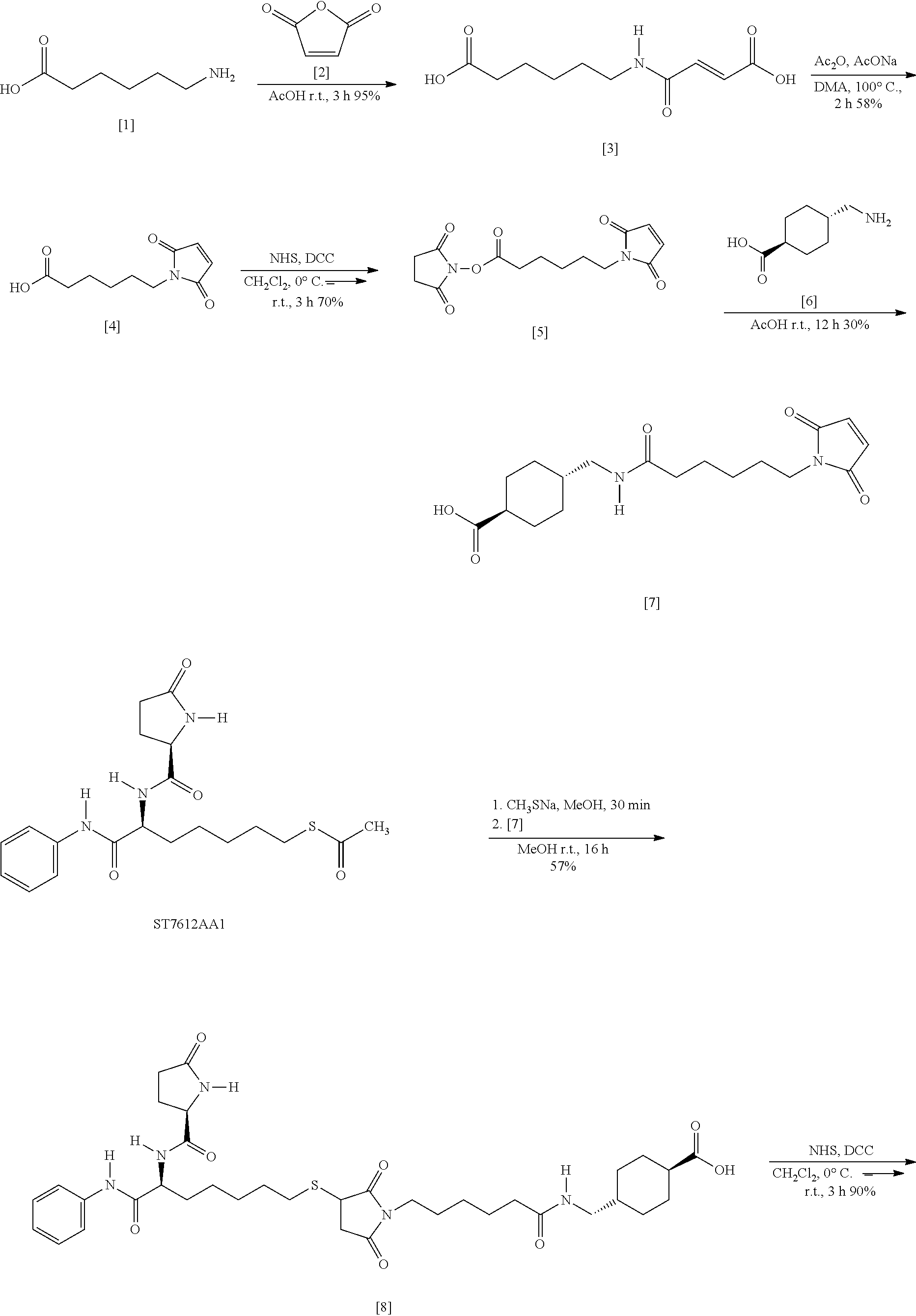

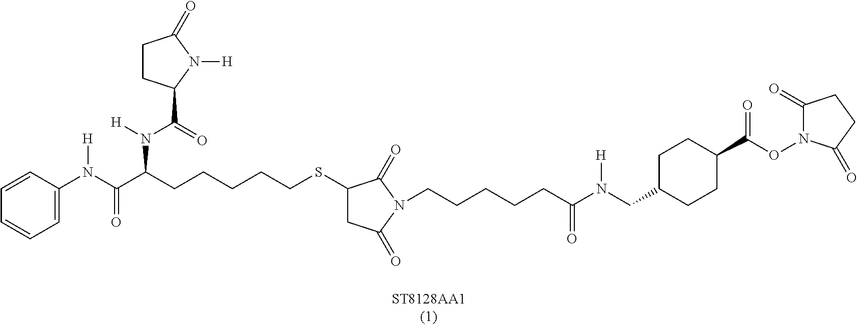

### (E)-6-(3-Carboxyacrylamido)hexanoic Acid [3]

A solution of maleic anhydride [2] (1.95 g, 19.9 mmol) in glacial acetic acid (10 mL) is added dropwise to a stirring solution of 6-aminocaproic acid [1] (2.6 g, 19.9 mmol) in glacial acetic acid (10 mL). The mixture is maintained at room temperature for 3 hours. The precipitate formed during the reaction is then filtrated, washed with diethyl ether in order to obtain compound [3] as a white solid, 4.3 g (95% yield). Any further purification of the solid is not required.

¹H NMR (400 MHz, DMSO) δ9.10 (s, 1H), 6.35 (d, J=12.6 Hz, 1H), 6.18 (d, J=12.6 Hz, 1H), 3.12 (m, 2H), 2.15 (t, J=7.4 Hz, 2H), 1.44 (m, 4H), 1.33-1.12 (m, 2H).

¹³C NMR (100 MHz, DMSO) δ 174.8, 172.4, 165.8, 133.5, 132.2, 33.9, 28.5, 26.3, 24.5, 21.4.

### 6-(2,5-Dioxo-2,5-dihydro-1H-pyrrol-1-yl)hexanoic Acid [4]

Acetic anhydride (3.8 mL, 40.75 mmol) and sodium acetate (418 mg, 5.09 mmol) are added at room temperature to a solution of compound [3] (4.3 g, 20.37 mmol) in dimethylacetamide (20 mL) under vigorous stirring and then heated to 100° C. for 2 hours. The mixture is then cooled to room temperature, diluted with dichloromethane (35 mL) and washed 5-6 times with HCl 0.5 M (6 portions of 10 mL each) to remove the dimethylacetamide. The organic layer is dried over anhydrous sodium sulfate, filtered and the solvent removed under vacuum to give product [4] as a colorless oil, 2.5 g (58% yield).

¹H NMR (400 MHz, MeOD) δ 9.87 (bs, 1H), 6.76 (d, J=1.6 Hz, 2H), 3.54-3.38 (m, 2H), 2.52-2.39 (m, 1H), 2.30-2.26 (m, 1H), 1.70-1.46 (m, 4H), 1.39-1.19 (m, 2H).

¹³C NMR (100 MHz, MeOD) δ 179.4, 171.1, 133.9, 36.9, 33.2, 29.2, 24.1, 23.3.

### 2,5-Dioxopyrrolidin-1-yl 6-(2,5-dioxo-2,5-dihydro-1H-pyrrol-1-yl)hexanoate [5]

Dicyclohexyl-carbodiimide (2.68 g, 13.02 mmol) and N-hydroxysuccinimide (1.36 g, 11.84 mmol) are added at room temperature to a stirrer solution of compound [4] (2.5 g, 11.84 mmol) in anhydrous dichloromethane (15 mL) and the mixture stirred at room temperature for 3 hours. The white solid is filtered with dichloromethane to remove the dicyclohexylurea, the organic phase is washed with HCl 0.1N and water, then dried over anhydrous sodium sulfate and the solvent removed by rotatory evaporation. The resulting residue is subjected to flash column chromatography with a medium pressure system Sepacore® Buchi (silica gel; gradient A: petroleum ether/B: ethyl acetate; B % 0-80 in 15 minutes) to give the activated acid [5] as a white solid, 2.4 g (70%). MS: m/z 309 [M+H]⁺.

¹H NMR (400 MHz, MeOD) δ 6.78 (s, 2H), 3.49-3.46 (m, 2H), 2.82 (s, 4H), 2.62-2.59 (m, 2H), 1.78-1.65 (m, 2H), 1.64-1.50 (m, 2H), 1.46-1.26 (m, 2H).

¹³C NMR (100 MHz, MeOD) δ 169.8, 169.0, 167.3, 132.5, 35.4, 28.6, 26.1, 23.8, 23.6, 22.3.

### 4-((6-(2,5-Dioxo-2,5-dihydro-1H-pyrrol-1-yl)hexanamido)methyl)cyclohexanecarboxylic Acid [7]

trans-4-(Aminomethyl)-cyclohexane carboxylic acid [6] (2.38 g, 15.17 mmol) is added at room temperature to a stirrer solution of compound [5] (2.46 g, 7.98 mmol) in acetic acid glacial (20 mL) and the reaction mixture is stirred at room temperature for 16 hours. The acetic acid is removed under reduced pressure, the residue diluted with dichloromethane and washed with water to eliminate the N-hydroxysuccinamide. The organic layer is dried over anhydrous sodium sulfate, filtered and the solvent removed under vacuum. The residue is subjected to flash chromatography with a medium pressure system Sepacore® Buchi, silica gel in gradient 0-100% of ethyl acetate in petroleum ether in 7 minutes, and 100% of ethyl acetate in 5 minutes, to give compound [7] as a white solid, 807 mg (30% yield). MS: m/z 349 [M−H]⁺.

¹H NMR (400 MHz, MeOD) δ 10.47 (bs, 1H), 7.81 (s, 1H), 6.66 (d, J=2.3 Hz, 2H), 3.35 (s, 2H), 2.88 (s, 2H), 2.04 (s, 2H), 1.86 (d, J=10.5 Hz, 2H), 1.68 (d, J=10.1 Hz, 2H), 1.47 (s, 2H), 1.36-1.03 (m, 8H), 0.85 (d, J=11.7 Hz, 2H).

¹³C NMR (100 MHz, MeOD) δ 176.8, 173.0, 169.5, 132.4, 43.4, 41.5, 35.9, 35.5, 33.8, 27.9, 26.6, 25.9, 24.4, 23.6.

### 4-((6-(2,5-Dioxo-3-((7-oxo-6-(5-oxopyrrolidine-2-carboxamido)-7-(phenylamino)heptyl)thio)pyrrolidin-1-yl)hexanamido)methyl)cyclohexanecarboxylic Acid (8)

A solution of sodium thiomethylate 1M in degassed methanol (17 mg, 0.25 mmol, 0.250 mL) is added at room temperature to a solution of ST7612AA1 (100 mg, 0.25 mmol) in degassed methanol. The stirring mixture is maintained at room temperature for 30 minutes. The reaction mixture is flushed with N₂ for 5-10 minutes to remove MeSH and compound 7 (88 mg, 0.25 mmol) is added. The mixture is kept at room temperature for 16 hours. The solvent is then removed by rotatory evaporation and the crude purified by column chromatography in gradient 2-20% methanol in dichloromethane. The compound (8) is obtained as a white solid, 102 mg (57%). MS: m/z 737 [M+Na]+.

¹H NMR (400 MHz, MeOD) δ 9.97 (bs, s), 7.92 (m 2H), 7.60 (m, 2H), 7.34 (m, 2H), 7.14 (m, 1H), 4.57 (m, 1H), 4.31 (m, 1H), 3.54 (m, 2H), 3.06 (d, J=4.7 Hz, 2H), 2.49-2.14 (m, 4H), 2.10-2.02 (m, 7H), 1.87-1.62 (m, 12H), 1.76-1.23 (m, 12H), 1.01 (d, J=12.4 Hz, 2H).

¹³C NMR (100 MHz, MeOD) δ 179.6, 178.4, 177.0, 175.3, 174.7, 173.0, 170.7, 128.0 (2C), 123.7, 119.5 (2C), 77.6, 56.2, 53.5, 44.6 (2C), 42.7, 38.6, 37.7, 35.5, 35.0, 31.4, 30.3, 29.1 (2C), 28.9, 28.0 (3C), 27.4, 26.4, 25.5, 25.0, 24.6, 24.4.

### 2,5-Dioxopyrrolidin-1-yl 4-((6-(2,5-dioxo-3-((7-oxo-6-(5-oxopyrrolidine-2-carboxamido)-7-(phenylamino)heptyl)thio)pyrrolidin-1-yl)hexanamido)methyl)cyclohexanecarboxylate (1), ST8128AA1

In a 50 mL flask under N₂ compound [8] (102 mg, 0.14 mmol) is dissolved in anhydrous dichloromethane (5 mL) containing 0.1 mL of dry dimethylformamide. N-Hydroxysuccinamide (24 mg, 0.21 mmol) and dicyclocarbodiimide (50 mg, 0.24 mmol) are added at room temperature and the reaction mixture is stirred at room temperature for 16 hours. The solvent is removed, and the residue purified by flash chromatography in 2-6% methanol in dichloromethane. The product ST8128AA1 is obtained as a colorless viscous liquid; 101 mg (90% yield). MS: m/z 811 [M+H]+; 833 [M+Na]⁺.

¹H NMR (400 MHz, CDCl₃) δ 9.43 (s, 1H), 7.78 (d, J=7.0 Hz, 1H), 7.54 (d, J=7.6 Hz, 2H), 7.35-7.14 (m, 2H), 7.08 (t, J=7.0 Hz, 1H), 6.12 (s, 1H), 4.67 (d, J=6.4 Hz, 1H), 4.23 (s, 1H), 3.69 (s, 1H), 3.48 (s, 2H), 3.17-2.93 (m, 3H), 2.83 (s, 5H), 2.26-2.03 (m, 9H), 1.97-1.71 (m, 8H), 1.71-1.18 (m, 16H), 1.02 (dd, J=24.0, 11.7 Hz, 2H).

¹³C NMR (100 MHz, CDCl₃) δ 178.9, 176.7, 176.4, 174.4, 173.2, 172.7, 170.5, 170.3, 168.9, 137.4, 128.48 (2C), 124.06, 119.58 (2C), 53.9, 45.4, 45.1, 42.1, 39.2, 38.7, 38.3, 37.6, 37.3, 36.3, 32.1, 31.3 (4C), 29.9, 28.8, 28.2, 27.2, 26.2, 25.3, 18.6, 17.3, 12.0.

### Example 2: Synthesis and Characterization of Payload (2), ST8152AA1

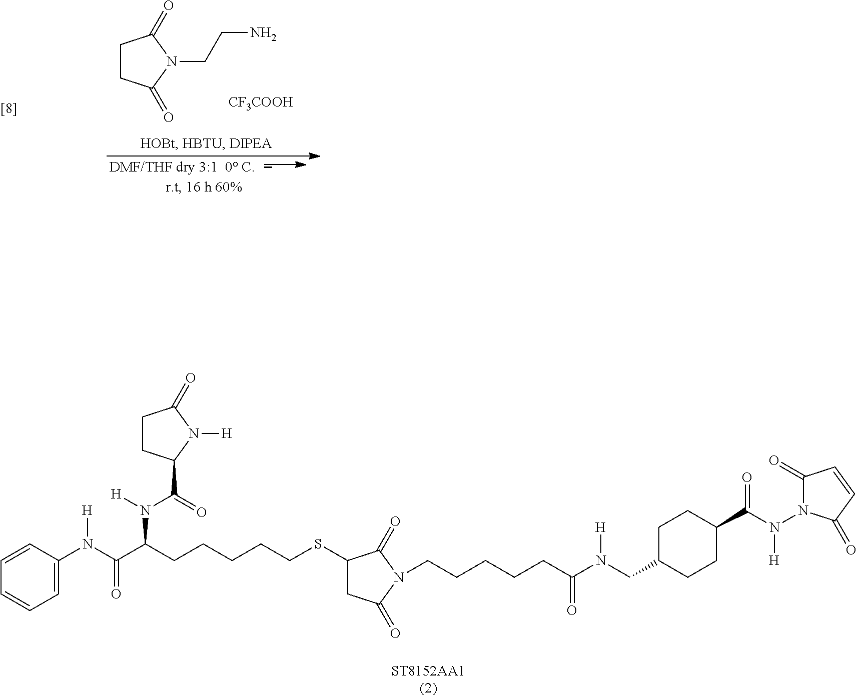

### N-(7-((1-(6-(((4-((2-(2,5-Dioxo-2,5-dihydro-1H-pyrrol-1-yl)ethyl)carbamoyl)cyclohexyl)methyl)amino)-6-oxohexyl)-2,5-dioxopyrrolidin-3-yl)thio)-1-oxo-1-(phenylamino)heptan-2-yl)-5-oxopyrrolidine-2-carboxamide (2), ST8152AA1

In a 50 mL flask under N₂ compound [8] (87 mg, 0.11 mmol) is dissolved in a mixture of tetrahydrofuran/dimethylformamide 3:1 (4 mL) at 0° C. Then N-(2-aminoethyl)maleimide trifluoroacetate salt (33 mg, 0.13 mmol), 1-hydroxybenzotriazole hydrate (22 mg, 0.16 mmol), HBTU (61 mg, 0.16 mmol) and N,N-diisopropylethylamine (0.048 mL, 0.27 mmol) are added and the mixture kept at 0° C. for 30 minutes, and then at room temperature for 16 hours. The reaction mixture is diluted with dichloromethane and washed two times with water and two times with brine, dried over anhydrous sodium sulfate and the solvent removed by rotatory evaporation. The residue is purified by flash column chromatography 2-20% methanol in dichloromethane. The product ST8152AA1 is obtained as a colorless viscous liquid, 55 mg (60% yield). MS: m/z 859 [M+Na]⁺.

¹H NMR (400 MHz, CDCl₃) δ 9.46 (bs, 1H), 7.60 (d, J=7.9 Hz, 2H), 7.36-7.23 (m, 2H), 7.09 (t, J=7.4 Hz, 1H), 6.72 (s, 2H), 6.49 (t, J=5.6 Hz, 1H), 6.32 (t, J=5.7 Hz, 1H), 4.65 (d, J=6.1 Hz, 1H), 4.37-4.24 (m, 1H), 3.83-3.60 (m, 2H), 3.60-3.37 (m, 5H), 3.25-2.97 (m, 4H), 2.89-2.61 (m, 2H), 2.60-2.11 (m, 4H), 2.00 (m, 5H), 1.90-1.72 (m, 13H), 1.72-1.15 (m, 8H), 0.94 (dd, J=24.3, 11.4 Hz, 2H).

### Example 3: Synthesis and Characterization of Payload (3), ST8132AA1

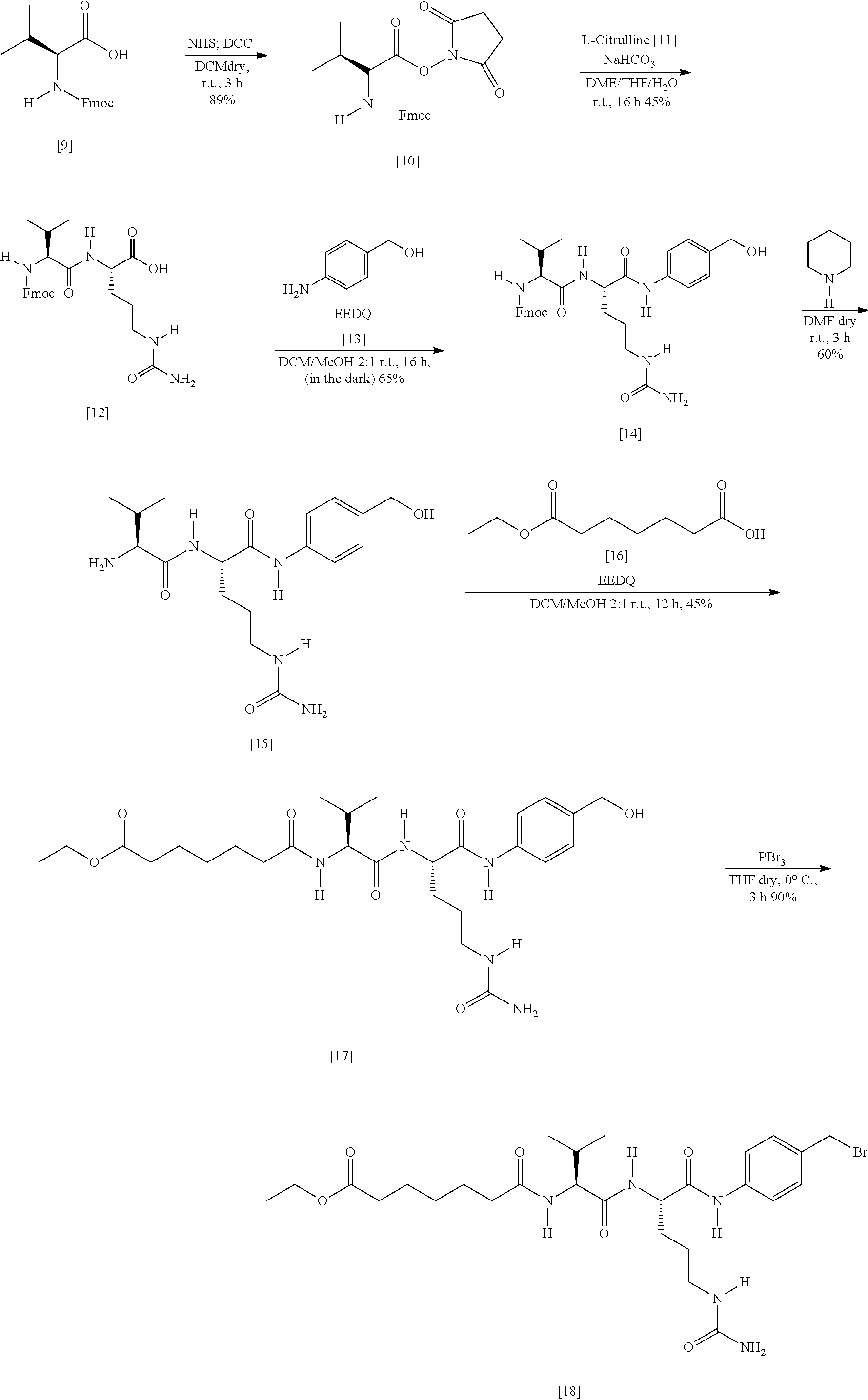

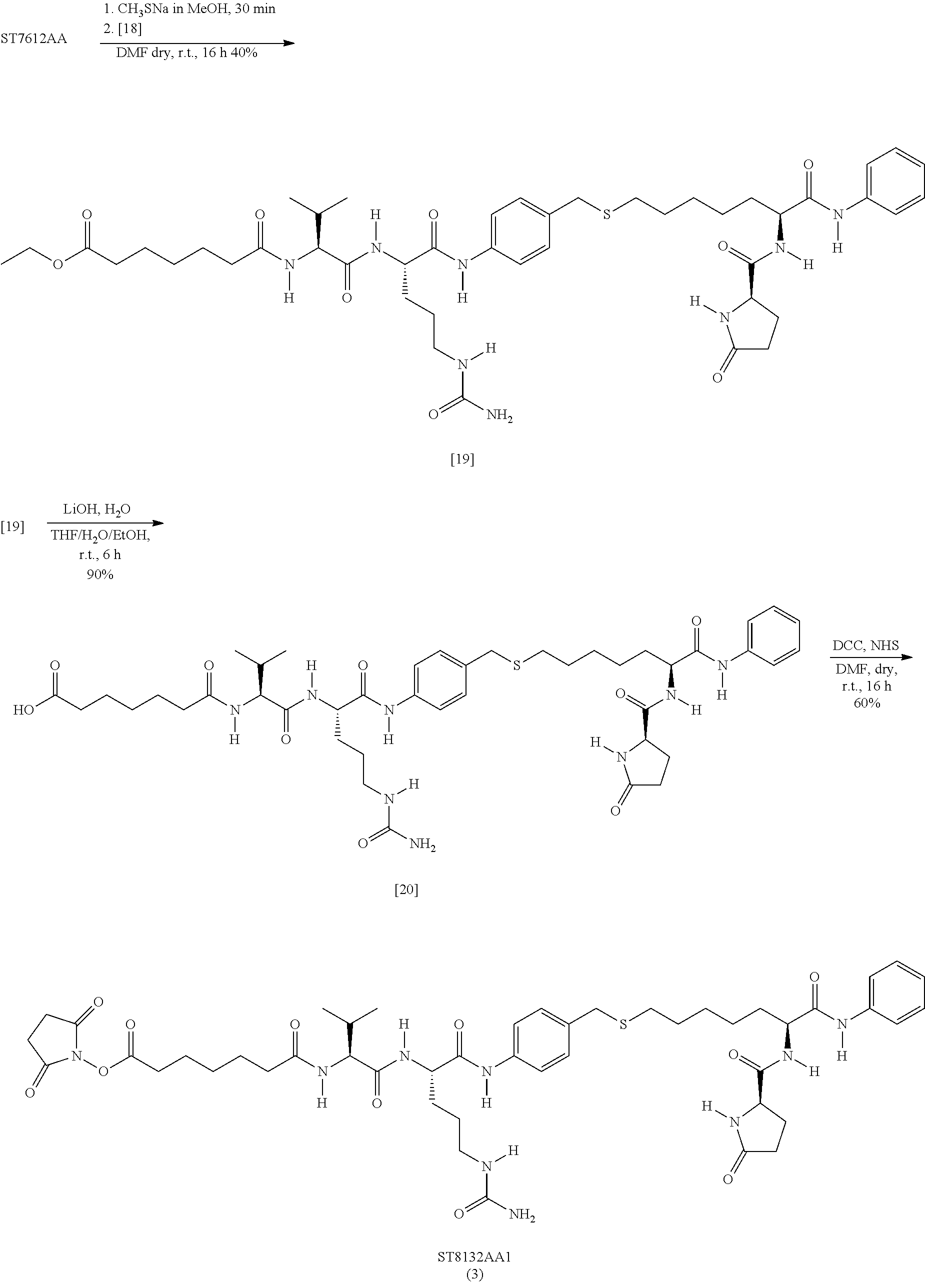 [SMILES: CCOC(=O)CCCCCC(=O)N[C@H](C(=O)N[C@@H](CCCNC(N)=O)C(=O)Nc1ccc(CSCCCCC[C@H](NC(=O)[C@H]2CCC(=O)N2)C(=O)Nc2ccccc2)cc1)C(C)C ; CC(C)[C@H](NC(=O)CCCCCC(=O)O)C(=O)N[C@@H](CCCNC(N)=O)C(=O)Nc1ccc(CSCCCCC[C@H](NC(=O)[C@H]2CCC(=O)N2)C(=O)Nc2ccccc2)cc1 ; CC(C)[C@H](NC(=O)CCCCCC(=O)ON1C(=O)CCC1=O)C(=O)N[C@@H](CCCNC(N)=O)C(=O)Nc1ccc(CSCCCCC[C@H](NC(=O)[C@H]2CCC(=O)N2)C(=O)Nc2ccccc2)cc1]

### (S)-2,5-Dioxopyrrolidin-1-yl 2-((((9H-fluoren-9-yl)methoxy)carbonyl)amino)-3-methylbutanoate [10]

Dicyclocarbodiimide (1.55 g, 7.52 mmol) and N-hydroxysuccinimide (762 mg, 6.63 mmol) are added at room temperature to a stirrer solution of Fmoc-Val-OH [9] (1.5 g, 4.42 mmol) in anhydrous dichloromethane (25 mL). The mixture is kept at room temperature for 3 hours. The white solid formed in this reaction is filtrated with dichloromethane to remove the dicyclohexylurea, the organic phase is washed with HCl 0.1N and water, then dried over anhydrous sodium sulfate and the solvent removed by rotatory evaporation. The residue is subjected to a flash column chromatography in 1% methanol in dichloromethane to afford product [10] as a white solid, 1.7 g (89% yield). MS: m/z 459 [M+Na]⁺.

¹H NMR (400 MHz, CDCl₃) δ 7.80 (d, J=7.5 Hz, 2H), 7.68-7.55 (m, 2H), 7.43 (t, J=7.4 Hz, 2H), 7.34 (dd, J=15.9, 8.5 Hz, 2H), 4.70 (d, J=4.6 Hz, 1H), 4.56-4.40 (m, 3H), 4.28 (t, J=6.6 Hz, 1H), 2.85 (s, 4H), 2.37 (dd, J=12.3, 6.5 Hz, 1H), 1.08 (dd, J=11.0, 6.9 Hz, 6H).

### (S)-2-((S)-2-((((9H-Fluoren-9-yl)methoxy)carbonyl)amino)-3-methylbutanamido)-5-ureidopentanoic Acid [12]

Compound [10] (1.7 g, 3.9 mmol) in dimethoxyethane (10 mL) is added to a solution of L-citrulline [11] (700 mg, 4 mmol) dissolved in a mixture of tetrahydrofuran and aqueous sodium bicarbonate (344 mg, 4 mmol in 10 mL of water). The reaction is stirred at room temperature for 16 hours. A solution of citric acid 15% in water (50 mL) is added and the mixture extracted with 10% isopropyl alcohol in ethyl acetate (2×75 mL). The solvent is removed by rotatory evaporation. After addition of diethyl ether and irradiation with ultrasounds, the formation of a solid is obtained. Filtration followed by washing with diethyl ether gave [12] as a white solid, 870 mg (45%). MS: m/z 495 [M−H]⁻.

¹H NMR (400 MHz, DMSO) δ 8.19 (bm 3H), 7.87 (t, J=25.1 Hz, 2H), 7.77 (t, J=6.8 Hz, 2H), 7.49-7.23 (m, 4H), 5.99 (s, 1H), 5.43 (s, 2H), 4.45-4.08 (m, 4H), 3.97 (t, J=7.5 Hz, 1H), 2.99 (d, J=5.1 Hz, 2H), 2.02 (d, J=6.1 Hz, 1H), 1.74 (s, 1H), 1.61 (d, J=7.5 Hz, 1H), 1.44 (s, 2H), 0.91 (dd, J=12.5, 6.3 Hz, 6H).

¹³C NMR (101 MHz, DMSO) δ 173.4, 171.3, 169.8, 167.8, 158.8, 156.0, 143.8, 140.7, 127.6, 127.0, 65.7, 59.8, 51.9, 46.7, 30.5, 26.6, 19.18 (2C).

### (9H-Fluoren-9-yl)methyl ((S)-1-(((S)-1-((4-(hydroxymethyl)phenyl)amino)-1-oxo-5-ureidopentan-2-yl)amino)-3-methyl-1-oxobutan-2-yl)carbamate [14]

EEDQ (840 mg, 3.4 mmol) is added to a solution containing compound [12] (870 mg, 1.7 mmol) and p-aminobenzyl alcohol (227 mg, 1.8 mmol) in dichloromethane/methanol 2:1 (15 mL). The reaction is left in the dark at room temperature for 16 hours. The solvents are removed, and the resulting solid residue filtrated using diethyl ether to give product [14] as a white solid, 660 mg (65% yield). MS: m/z 624 [M+Na]⁺.

¹H NMR (400 MHz, DMSO) δ 10.00 (bs, 1H), 8.13 (m, 4H), 7.92 (d, J=7.3 Hz, 2H), 7.76 (d, J=7.6 Hz, 2H), 7.58 (d, J=8.1 Hz, 2H), 7.51-7.10 (m, 2H), 6.02 (s, 1H), 5.43 (m, 4H), 5.13 (s, 1H), 4.47 (s, 3H), 4.31 (m 4H), 3.13-2.74 (m, 2H), 2.03 (s, 1H), 1.83-1.55 (m, 2H), 1.43 (s, 2H), 0.90 (d, J=6.7 Hz, 6H).

¹³C NMR (101 MHz, DMSO) δ 171.2, 170.4, 158.93, 156.15, 144.6, 143.8, 140.7, 137.5, 127.6, 127.2 (2C) 125.3, 120.1 (2C), 118.9, 65.7, 62.6, 60.1, 53.0, 46.7, 31.0, 30.5, 26.7, 19.6, 18.7.

### (S)-2-((S)-2-Amino-3-methylbutanamido)-N-(4-(hydroxymethyl)phenyl)-5-ureidopentanamide [15]

Piperidine (2.2 mL) is added to a solution of compound [14] (660 mg, 1.09 mmol) in anhydrous dimethylformamide (5 mL) and the reaction left at room temperature for 3 h. The solvent is removed under vacuum and the residue treated with dichloromethane in order to obtain a solid that is filtered to give product [15], 240 mg (60% mg). MS: m/z 380 [M+H]⁺.

¹H NMR (400 MHz, DMSO) δ 10.11 (s, 1H), 8.25 (s, 1H), 7.58 (d, J=8.2 Hz, 2H), 7.27 (d, J=8.2 Hz, 2H), 6.10 (s, 1H), 5.46 (s, 2H), 4.48 (m, 3H), 3.23-3.09 (m, 2H), 3.11-2.88 (m, 2H), 1.99 (dd, J=12.2, 6.4 Hz, 2H), 1.99 (dd, J=12.2, 6.4 Hz, 1H), 1.81-1.55 (m, 2H), 1.55-1.28 (m, 2H), 1.04-0.68 (m, 6H).

¹³C NMR (101 MHz, DMSO) δ 173.3, 170.4, 158.9, 137.5, 126.9 (2C), 125.3, 119.9 (2C), 62.6, 59.3, 52.6, 31.1, 29.9, 26.6, 19.3, 17.0 (2C).

### Ethyl 7-(((S)-1-(((S)-1-((4-(hydroxymethyl)phenyl)amino)-1-oxo-5-ureidopentan-2-yl)amino)-3-methyl-1-oxobutan-2-yl)amino)-7-oxoheptanoate [17]

EEDQ (311 mg, 1.26 mmol) is added to a solution of compound [15] (240 mg, 0.63 mmol) and monoethyl pimelate [16] (0.123 mL, 0.69 mmol) in a mixture of dichloromethane/methanol 2:1 (20 mL). The reaction is left in the dark at room temperature for 12 hours. The solvents are removed and the residue purified by column chromatography 2-20% methanol in dichloromethane to give product [17] as a viscous solid, 170 mg (45% yield). MS: m/z 572 [M+Na]⁺.

¹H NMR (400 MHz, MeOD) δ 7.59 (d, J=8.3 Hz, 2H), 7.34 (d, J=8.3 Hz, 2H), 4.65-4.44 (m, 4H), 4.25 (d, J=7.4 Hz, 1H), 4.15 (dd, J=14.2, 7.1 Hz, 2H), 3.30-3.06 (m, 2H), 2.34 (dd, J=13.9, 6.7 Hz, 2H), 2.13 (d, J=6.9 Hz, 1H), 1.92 (dd, J=14.1, 6.2 Hz, 2H), 1.89-1.74 (m, 2H), 1.74-1.34 (m, 12H), 1.30 (dd, J=18.0, 10.9 Hz, 3H), 1.06-0.84 (m, 8H).

¹³C NMR (101 MHz, MeOD) δ 174.5, 172.1, 171.7, 170.4, 158.9, 136.5, 126.8 (2C), 124.1, 120.9 (2C), 62.9, 59.5, 58.6, 53.0, 38.4, 34.7, 33.0, 29.8, 29.2, 27.9, 27.2, 25.0, 23.6, 17.5, 16.9, 12.7.

### Ethyl 7-(((S)-1-(((S)-1-((4-(bromomethyl)phenyl)amino)-1-oxo-5-ureidopentan-2-yl)amino)-3-methyl-1-oxobutan-2-yl)amino)-7-oxoheptanoate [18]

Phosphorus tribromide (0.043 mL, 0.46 mmol) is added at 0° C. to a solution of compound [17] (170 mg, 0.31 mmol) in anhydrous tetrahydrofuran (5 mL). The mixture is kept at 0° C. for 3 hours. The reaction mixture is directly submitted to a silica column for the purification by flash chromatography 1-6% methanol in dichloromethane to give the product [18] as a white solid, 170 mg (90% yield). MS: m/z 613[M+H]⁺; 635 [M+Na]⁺.

¹H NMR (400 MHz, Acetone-d6) δ 9.85 (d, J=6.1 Hz, 1H), 7.85 (dd, J=25.2, 8.1 Hz, 2H), 7.39 (dt, J=44.7, 22.3 Hz, 2H), 4.90-4.54 (m, 5H), 4.19-3.97 (m, 2H), 3.58 (t, J=6.6 Hz, 1H), 3.36 (d, J=20.6 Hz, 2H), 2.41 (dd, J=21.3, 6.3 Hz, 2H), 2.33-2.16 (m, 3H), 2.14-2.02 (m, 2H), 2.04-1.93 (m, 2H), 1.93-1.73 (m, 2H), 1.71-1.46 (m, 2H), 1.48-1.13 (m, 6H), 1.11-0.77 (m, 8H).

### Ethyl 7-(((S)-3-methyl-1-oxo-1-(((S)-1-oxo-1-((4-((((S)-7-oxo-6-((R)-5-oxo-pyrrolidine-2-carboxamido)-7-(phenylamino)heptyl)thio)methyl)phenyl)amino)-5-ureidopentan-2-yl)amino)butan-2-yl)amino)-7-oxoheptanoate [19]

ST7612AA1 (125 mg, 0.31 mmol) is dissolved in degassed methanol (8 mL) in a 50 mL flask under N₂ atmosphere. A solution of sodium thiomethylate 1M in degassed methanol (22 mg, 0.31 mmol) is added at room temperature to the first solution. The stirring mixture is maintained at room temperature for 30 minutes. The solution is flushed with N₂ for 5-10 minutes to eliminate MeSH, then the solvent is removed by rotatory evaporation. Compound [18] (170 mg, 0.31 mmol) dissolved in a minimum amount of anhydrous dimethylformamide is added to the residue and the mixture kept at room temperature for 16 hours. The solvent is removed by rotatory evaporation and the crude purified by column chromatography, 1-40% methanol in dichloromethane to give [19] as an amorphous solid; 106 mg (40% yield). MS: m/z 896 [M+H]⁺: 918 [M+Na]⁺.

¹H NMR (400 MHz, MeOD) δ 7.60 (s, 4H), 7.43-7.18 (m, 4H), 7.13 (s, 1H), 4.55 (m, 4H), 4.40 (m, 2H), 4.13 (m, 2H), 3.66 (m, 6H), 3.19 (s, 4H), 2.75 (s, 2H), 2.37 (m, 6H), 2.11 (s, 2H), 1.97 (s, 1H), 1.82 (s, 2H), 1.64 (s, 7H), 1.42 (s, 4H), 1.27 (s, 4H), 1.01 (s, 6H).

### 7-(((S)-3-Methyl-1-oxo-1-(((S)-1-oxo-1-((4-((((S)-7-oxo-6-((R)-5-oxopyrrolidine-2-carboxamido)-7-(phenylamino)heptyl)thio)methyl)phenyl)amino)-5-ureidopentan-2-yl)amino)butan-2-yl)amino)-7-oxoheptanoic acid [20]

Lithium hydroxide monohydrate (42 mg, 1 mmol) is added to a solution of [19] (300 mg, 0.33 mmol) in a mixture of tetrahydrofuran/water/ethanol 1:1:1 (12 mL). The reaction is kept at room temperature for 6 hours, then it is diluted with ethyl acetate and washed with HCl 1N. The crude (180 mg) is directly used for the next step without any purification. MS: m/z 874 [M+Li]⁺; 890 [M+Na]⁺.

### 2,5-Dioxopyrrolidin-1-yl 7-(((S)-3-methyl-1-oxo-1-(((S)-1-oxo-1-((4-((((S)-7-oxo-6-((R)-5-oxopyrrolidine-2-carboxamido)-7-(phenylamino)heptyl)thio)methyl)phenyl)amino)-5-ureidopentan-2-yl)amino)butan-2-yl)amino)-7-oxoheptanoate (3), ST8132AA1

Dicyclohexylcarbodiimide (70 mg, 0.35 mmol) and N-hydroxysuccinimide (34 mg, 0.3 mmol) are added at room temperature to a stirrer solution of compound [20](180 mg, 0.20 mmol) in anhydrous dimethylformamide (3 mL). The mixture is kept at room temperature for 16 hours. The white solid formed in this reaction is filtrated to remove the dicyclohexylurea, the organic phase is washed with HCl 0.1N and water, then dried over anhydrous sodium sulfate and the solvent removed under vacuum. The resulting residue is subjected to flash column chromatography 2-20% methanol in dichloromethane to give the activated acid (3) as a white viscous solid, 109 mg (60% yield). MS: m/z 987 [M+Na]⁺.

¹H NMR (400 MHz, DMSO) δ 10.10 (s, 4H), 9.95 (s, 1H), 7.59 (m, 4H), 7.33 (t, J=7.5 Hz, 2H), 7.24 (d, J=7.8 Hz, 2H), 7.08 (t, J=7.2 Hz, 1H), 6.00 (s, 1H), 5.42 (s, 2H), 4.43 (d, J=14.3 Hz, 2H), 3.67 (s, 1H), 3.12-2.90 (m, 2H), 2.84 (s, 4H), 2.65 (m, 5H), 2.53 (s, 6H), 2.17 (m, 2H), 1.63 (m, 10H), 1.36 (s, 4H), 0.94-0.75 (m, 8H).

### Example 4: Synthesis and Characterization of Payload (4), ST8190AA1

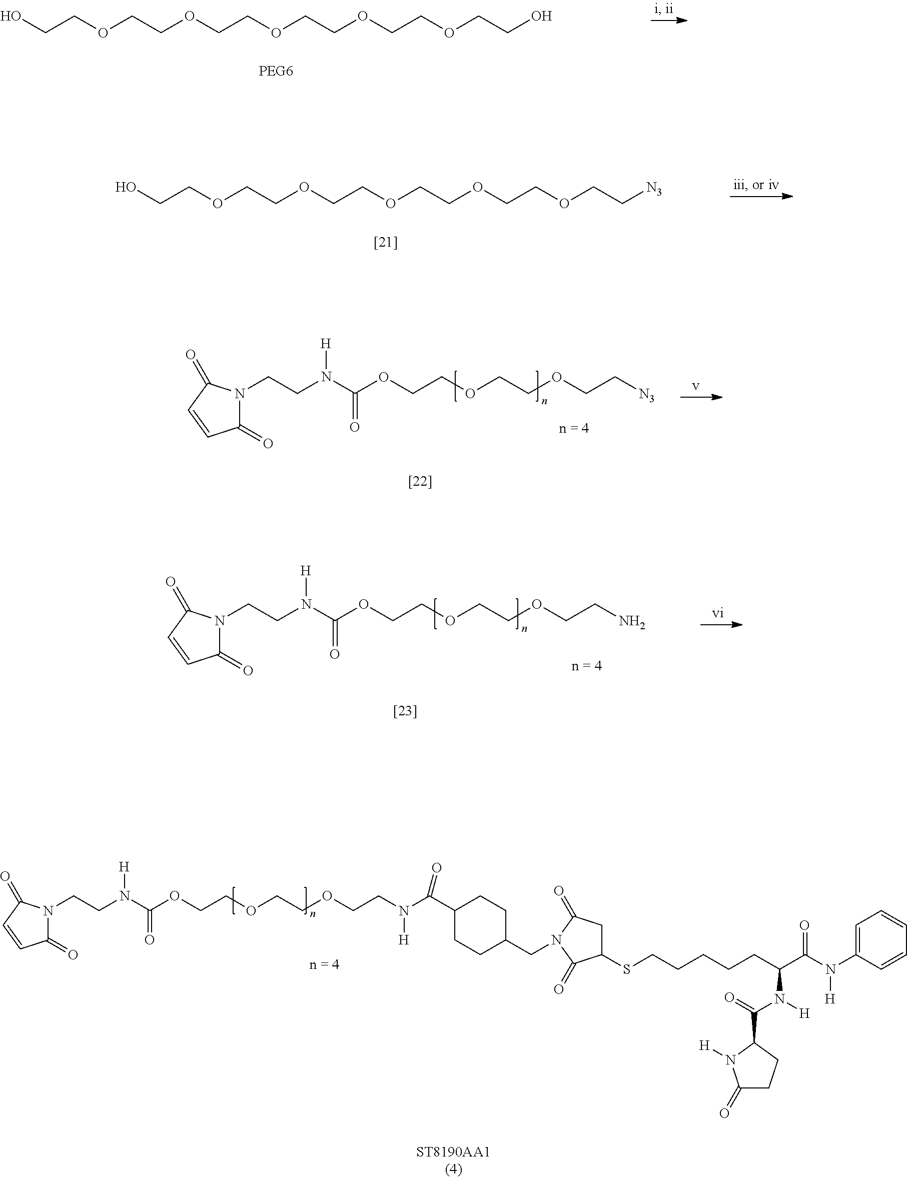

i. Tosyl-Cl, TEA, THF; ii. NaN₃, DMF; iii. p-nitrophenylchloroformate, DMAP, DCM; iv. N-(2-aminoethyl)maleimide trifluoroacetate, DBU, DCM; v. PPh₃, THF; vi. 4-((2,5-dioxo-3-(((6S)-7-oxo-6-(5-oxopyrrolidine-2-carboxamido)-7-(phenylamino)heptyl)thio)pyrrolidin-1-yl)methyl)cyclohexane-1-carboxylic acid HOBt, HBtU, DIPEA, THF.

### 2[-2-(2-{2-[2-(2-Hydroxyethoxy)-ethoxy]-ethoxy}-ethoxy)-ethoxy]-ethylazide [21]

To a solution of hexaethyleneglycol (50 g, 177 mmol) and triethylamine (14 mL, 100 mmol) in THF (250 mL) para-toluensulfonyl-chloride (13 g, 70 mmol) in THF (250 mL) is added and the solution is stirred overnight. The solution is then diluted with CH₂Cl₂ (200 mL) washed with 1N HCl (3×150 mL) and brine (150 mL) and dried over Na₂SO₄. After filtration and evaporation of the solvents in vacuo the residue is purified by column chromatography (silica, 2% methanol in CH₂Cl₂) to give the monotosyl derivative (24.4 g, 56 mmol, 80% compared to p-TsCl) as colourless oil: TLC (CH₂Cl₂:MeOH 95:5): Rf=0.32.

¹HNMR (400 MHz, CDCl3) δ=7.79 (d, J=8.0 Hz, 2H), 7.33 (d, J=8.0 Hz, 2H), 4.09-4.07 (m, 2H), 3.74-3.56 (m, 22H), 2.97 (br, 1H, OH), 2.44 (s, 3H); ¹³C NMR (100 MHz, CDCl3) δ=145.0, 133.3, 130.0, 128.2, 72.7, 70.9-70.5, 69.5, 68.9, 61.9, 21.8; MS: m/z 437 [M+H]⁺.

Then a mixture of the tosylderivative (20.4 g, 56 mmol) and NaN₃ (3.7 g, 57 mmol) in DMF (150 mL) is stirred at room temperature overnight. DMF is evaporated in vacuo and the residue dissolved in AcOEt, and filtered over Celite®. The AcOEt is evaporated under reduced pressure to yield pure [21] (14.6 g, 48 mmol, 85%): TLC (CH₂Cl₂:MeOH 94:6): Rf=0.38; ¹H NMR (400 MHz, CDCl₃) δ=3.72-3.57 (m, 22H), 3.38-3.36 (m, 2H), 2.80 (bs, 1H); ¹³C NMR (100 MHz, CDCl₃) δ=72.8, 70.7-70.1, 61.8, 50.7.

### 17-Azido-3,6,9,12,15-pentaoxaheptadecyl(2-(2,5-dioxo-2,5-dihydro-1H-pyrrol-1-yl)ethyl)carbamate [22]

To a solution of [21] (500 mg, 1.63 mmol) in CH₂Cl₂ (50 ml) and DMAP (1 g, 9 mmol), 4-nitrophenylchloroformiate (1.15 g, 5.7 mmol) is added at 0° C. The mixture is stirred at room temperature 2 h. The solution is then diluted with CH₂Cl₂ (50 mL) washed with 1N HCl (3×25 mL) and brine (50 mL) and dried over Na₂SO₄ dry. After filtration and evaporation of the solvents in vacuo the residue is purified by column chromatography (CH₂Cl₂:MeOH 95:5). A yellow solid is isolated (501 mg g, 61%): Rf=0.75; ¹H NMR (400 MHz, CDCl₃) δ=8.23 (dd, J=9.6, 2.8 Hz, 2H), 7.35 (dd, J=9.5, 2.9 Hz, 2H), 4.43-4.35 (m, 2H), 3.81-3.74 (m, 2H), 3.74-3.52 (m, 24H), 3.34 (dd, J=6.6, 3.5 Hz, 2H); MS: m/z 471 [M−H]⁻. The para-nitro derivative (501 mg, 1 mmol) and DBU (1.5 ml, 10 mmol) are dissolved in CH₂Cl₂ and N-(2-aminoethyl)maleimide trifluoroacetate (254 mg, 1 mmol) is added. The reaction mixture is stirred at room temperature overnight. The solution is then diluted with CH₂Cl₂ (25 mL) washed with 1N HCl (3×10 mL) and brine (20 mL) and dried over Na₂SO₄ dry. After filtration and evaporation of the solvents in vacuo the residue is purified by column chromatography (CH₂Cl₂:MeOH 95:5) obtaining a white solid (47 mg, 0.1 mmol, 10%). ¹H NMR (400 MHz, CDCl₃) δ: 7.80 (s, 1H), 3.92-3.50 (m, 24H), 3.38-3.36 (m, 4H); ¹³C NMR (100 MHz, CDCl₃) δ=171.2, 157.2, 135.6, 72.8, 70.7-70.1, 61.8, 50.7. MS: m/z 474 [M+H]⁺.

### 17-Amino-3,6,9,12,15-pentaoxaheptadecyl (2-(2,5-dioxo-2,5-dihydro-1H-pyrrol-1-yl)ethyl)carbamate [23]

A solution of [22] (47 mg, 0.1 mmol) in dry THF (10 mL) is cooled to 0° C. Triphenyl phosphine (53 g, 0.2 mmol) is added, and the mixture is stirred for 24 h at room temperature. H₂O (5 mL) is then added to hydrolyse the intermediate phosphorus adducts and the solution is stirred for another 24 h at room temperature. THF is evaporated and the solid residue is suspended in water (10 mL). The insoluble salts are filtered, and the filtrate washed with toluene (5×5 mL) and evaporated to yield [23] (44 mg, 0.1 mmol, 99%) as pale yellow oil, which is used in the next step without further purification. ¹H NMR (400 MHz, CDCl₃) δ=7.80 (s, 1H), 3.92-3.50 (m, 22H), 3.38-3.36 (m, 4H) 3.01-3.11 (m, 2H), 1.81 (bs, 2H); MS: m/z 448 [M+H]⁺.

### 1-((1S,4R)-4-((2,5-Dioxo-3-(((6S)-7-oxo-6-(5-oxopyrrolidine-2-carboxamido)-7-(phenylamino)heptyl)thio)pyrrolidin-1-yl)methyl)cyclohexyl)-1-oxo-5,8,11,14,17-pentaoxa-2-azanonadecan-19-yi (2-(2,5-dioxo-2,5-dihydro-1H-pyrrol-1-yl)ethyl)carbamate (4), ST8190AA1

4-((2,5-Dioxo-3-(((6S)-7-oxo-6-(5-oxopyrrolidine-2-carboxamido)-7-(phenylamino)heptyl)thio)pyrrolidin-1-yl)methyl)cyclohexane-1-carboxylic acid (60 mg, 0.1 mmol) is dissolved in dry THF (15 mL) and the reaction mixture cooled down to 0° C. HOBt (20 mg, 0.15 mmol), HBTU (57 mg, 0.15 mmol), [23] (44 mg, 0.1 mmol) and DIPEA (48 μL, 0.25 mmol) are added and the reaction mixture stirred at room temperature overnight. The solution is then diluted with AcOEt (15 mL) and extracted with H₂O (3×10 mL). The organic phases are dried over Na₂SO₄ dry and after filtration and evaporation of the solvents in vacuo the residue is purified by column chromatography (CH₂Cl₂:MeOH 98:2) obtaining ST8190AA1 as a colourless oil (31 mg, 0.03 mmol, 10%). MS: m/z 1031 [M+H]⁺.

### Example 5: Synthesis and Characterization of Payload (5), ST8189AA1

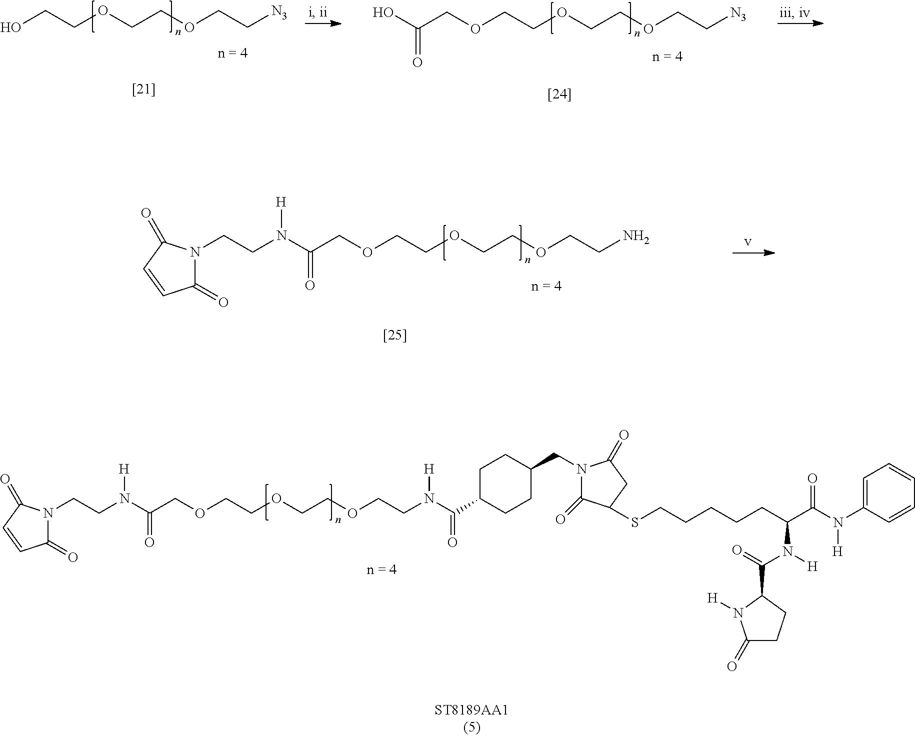

i. EtOCOCH₂Br, NaH, THF; ii. LiOH, THF, H₂O, EtOH; iii. N-(2-aminoethyl)maleimide trifluoroacetate, DBU, DCM; iv. PPh₃, THF; v. 4-((2,5-dioxo-3-(((6S)-7-oxo-6-(5-oxopyrrolidine-2-carboxamido)-7-(phenylamino)heptyl)thio)-pyrrolidin-1-yl)methyl)cyclohexane-1-carboxylic acid, HOBt, HBtU, DIPEA, THF.

### 20-Azido-3,6,9,12,15,18-hexaoxaicosanoic Acid [24]

NaH 60% wt in mineral oil (29 mg, 0.39 mmol) is suspended in dry THF (5 mL) and a solution of [21] (100 mg, 0.32 mmol) in dry THF (3 mL) is slowly added at 0° C. After stirring for 20 min at 0° C., a solution of ethyl-bromoacetate (53 μL, 0.48 mmol) in dry THF (3 mL) is added and the mixture stirred at room temperature over-night. H₂O (1 mL) is slowly added and the solvent evaporated under reduced pressure. The solid obtained is purified by flash chromatography (CH₂Cl₂:MeOH 95:5) giving a pale rose oil in quantitative yields (51 mg, 0.13 mmol). ¹H NMR (400 MHz, CDCl₃) δ=4.28 (s, 2H); 4.21 (q, 2H); 3.92-3.50 (m, 22H), 3.38-3.36 (m, 4H), 2.05-1.98 (m, 2H); 1.23 (t, 3H); MS: m/z 394 [M+H]⁺. The ethyl ester (51 mg, 0.13 mmol) is dissolved in a a 1:1:1 mixture of H₂O, THF and EtOH (15 mL) and LiOH monohydrate is added (16 mg, 0.39 mmol). The mixture is stirred at reflux for 2 h, then HCl added until pH 7. The solvents are evaporated under reduced pressure and the solid obtained washed with EtOH (2×10 mL). The solution is evaporated arising a colourless oil of [24] (47 mg, 0.13 mmol), directly used for the next step. MS: m/z 364 [M+H]⁺.

### 20-Amino-N-(2-(2,5-dioxo-2,5-dihydro-1H-pyrrol-1-yl)ethyl)-3,6,9,12,15,18-hexaoxaicosanamide [25]

To a solution of [24] (47 mg, 0.13 mmol) is dissolved in dry THF (15 mL) and the reaction mixture cooled down to 0° C. HOBt (26 mg, 0.2 mmol), HBtU (76 mg, 0.2 mmol), N-(2-aminoethyl)maleimide trifluoroacetate (33 mg, 0.13 mmol) and DIPEA (63 μL, 0.33 mmol) are added and the reaction mixture stirred at room temperature over-night. The solution is then diluted with AcOEt (15 mL) and extracted with H₂O (3×10 mL). The organic phases are dried over Na₂SO₄ dry and after filtration and evaporation of the solvents in vacuo the residue is purified by column chromatography (CH₂Cl₂:MeOH 98:2) obtaining a colourless oil (49 mg, 0.10 mmol). MS: m/z 488 [M+H]⁺.

The azido derivative obtained (49 mg, 0.10 mmol) is dissolved in dry THF (10 mL) at 0° C. Triphenyl phosphine (53 g, 0.2 mmol) is added and the mixture is stirred for 24 h at room temperature. H₂O (5 mL) is then added to hydrolyse the intermediate phosphorus adducts and the solution is stirred for another 24 h at room temperature. THF is evaporated and the solid residue is suspended in water (10 mL). The insoluble salts are filtered, and the filtrate washed with toluene (5×5 mL) and evaporated to yield [25] (46 mg, 0.1 mmol, 99%) as a pale yellow oil, which is used in the next step without further purification. ¹H NMR (400 MHz, CDCl₃) δ=8.03 (bs, 1H); 7.80 (s, 1H), 4.26 (s, 2H); 3.92-3.50 (m, 26H), 3.03-3.07 (m, 2H), 1.81 (bs, 2H); MS: m/z 462 [M+H]+.

### N-((2S)-7-((1-((4-((1-(2,5-Dioxo-2,5-dihydro-1H-pyrrol-1-yl)-4-oxo-6,9,12,15,18,21-hexaoxa-3-azatricosan-23-yl)carbamoyl)cyclohexyl)methyl)-2,5-dioxopyrrolidin-3-yl)thio)-1-oxo-1-(phenylamino)heptan-2-yl)-5-oxopyrrolidine-2-carboxamide (5), ST8189AA1

4-((2,5-Dioxo-3-(((6S)-7-oxo-6-(5-oxopyrrolidine-2-carboxamido)-7-(phenylamino) heptyl)thio)pyrrolidin-1-yl)methyl)cyclohexane-1-carboxylic acid (60 mg, 0.1 mmol) is dissolved in dry THF (15 mL) and the reaction mixture cooled down to 0° C. HOBt (20 mg, 0.15 mmol), HBtU (57 mg, 0.15 mmol), [25] (46 mg, 0.1 mmol) and DIPEA (48 μL, 0.25 mmol) are added and the reaction mixture stirred at room temperature over-night. The solution is then diluted with AcOEt (15 mL) and extracted with H₂O (3×10 mL). The organic phases are dried over Na₂SO₄ dry and after filtration and evaporation of the solvents in vacuo the residue is purified by column chromatography (CH₂Cl₂:MeOH 98:2) obtaining the payload ST8189AA1 as a colourless oil (21 mg, 0.02 mmol, 20%). MS: m/z 1045 [M+H]⁺.

### Example 6: Synthesis and Characterization of Payload (6), ST8191AA1

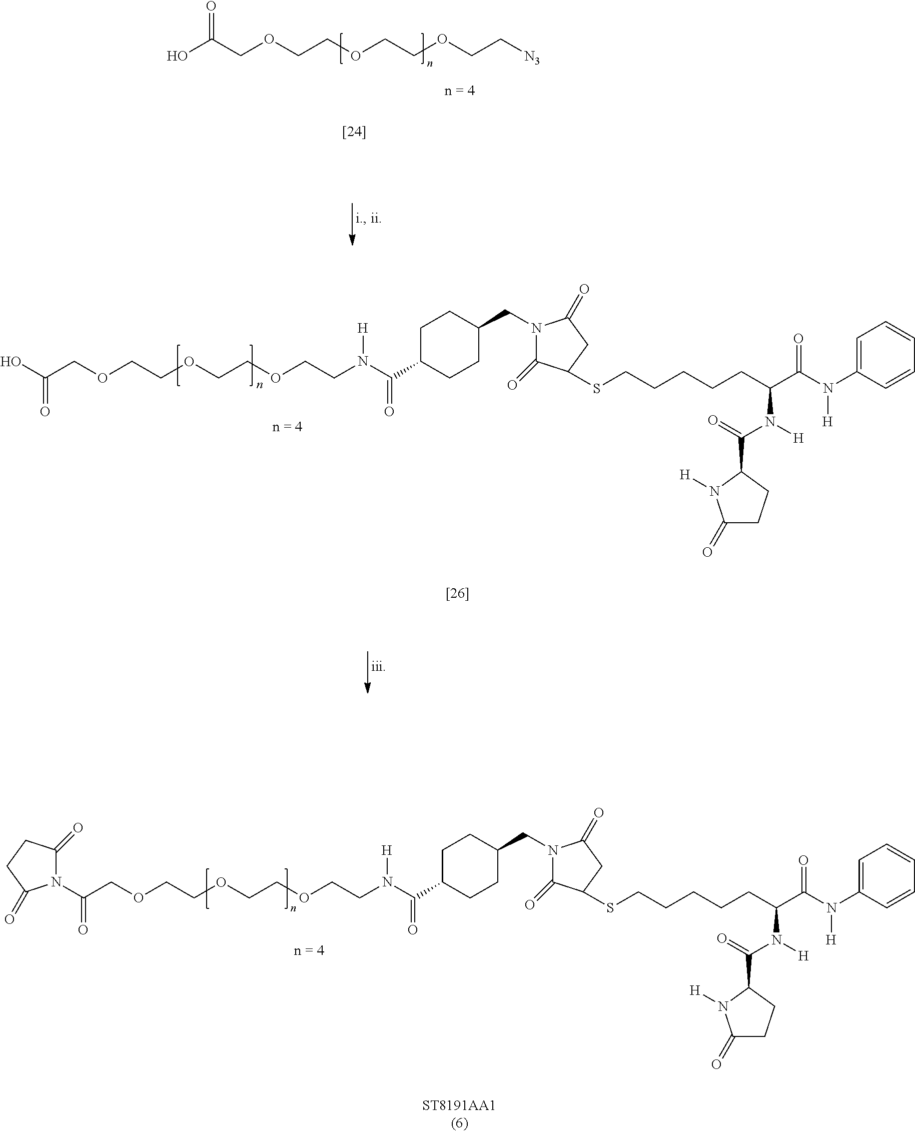

i.PPh₃, THF; ii. 4-((2,5-dioxo-3-(((6S)-7-oxo-6-(5-oxopyrrolidine-2-carboxamido)-7-(phenylamino)heptyl)thio)-pyrrolidin-1-yl)methyl)cyclohexane-1-carboxylic acid, DCC, NHS, DCM; iii. DCC, NHS, DCM.

### 1-(4-((2,5-Dioxo-3-(((6S)-7-oxo-6-(5-oxopyrrolidine-2-carboxamido)-7-(phenylamino)heptyl)thio)pyrrolidin-1-yl)methyl)cyclohexyl)-1-oxo-5,8,11,14,17,20-hexaoxa-2-azadocosan-22-oic Acid [26]

Azide [24] (49 mg, 0.10 mmol) is dissolved in dry THF (10 mL) at 0° C. Triphenyl phosphine (53 mg, 0.2 mmol) is added and the mixture stirred for 24 h at room temperature. H₂O (5 mL) is then added to hydrolyse the intermediate phosphorus adducts and the solution stirred for another 24 h at room temperature. THF is evaporated and the solid residue suspended in water (10 mL). The insoluble salts are filtered, and the filtrate washed with toluene (5×5 mL) and evaporated to yield 20-amino-3,6,9,12,15,18-hexaoxaicosanoic acid (46 mg, 0.1 mmol, 99%) as pale yellow oil, which is used in the next step without further purification. ¹H NMR (400 MHz, CD₃OD) δ=12.0 (bs, 1H); 4.26 (s, 2H); 3.92-3.50 (m, 22H), 3.03-3.07 (m, 2H), 1.81 (bs, 2H); MS: m/z 340 [M+H]⁺. 4-((2,5-Dioxo-3-(((6S)-7-oxo-6-(5-oxopyrrolidine-2-carboxamido)-7-(phenylamino)heptyl)thio)-pyrrolidin-1-yl)methyl)cyclohexane-1-carboxylic acid, (80 mg, 0.11 mmol), in dry CH₂Cl₂ (5 mL), is added, at room temperature to a solution containing dicyclohexyl-carbodiimide (0.11 mmol) and N-hydroxysuccinimide (0.1 mmol), in dry CH₂Cl₂ and the mixture stirred at room temperature for 3 hours. The white solid is filtered with dichloromethane to remove the dicyclohexylurea, the organic phase is washed with HCl 0.1N and H₂O, then dried over dry sodium sulfate and the solvent removed under reduced pressure. The resulting residue is purified by flash column chromatography to give the activated acid as a white waxy material (yield 90%) that is dissolved into dimethoxyethane (5 mL) and treated with 20-amino-3,6,9,12,15,18-hexaoxaicosanoic acid (46 mg, 0.1 mmol) dissolved in a mixture of tetrahydrofuran and aqueous sodium bicarbonate (15 mg, 0.15 mmol in 2 mL of water). The reaction is stirred at room temperature for 16 hours. A solution of citric acid 15% in water (2.5 mL) is added and the mixture extracted with 10% isopropyl alcohol in ethyl acetate (2×5 mL). The solvent is removed by rotatory evaporation. After addition of diethyl ether and irradiation with ultrasounds, the formation of a solid is obtained. Filtration followed by washing with diethyl ether gave [26] as a white solid, 65 mg (70%). MS: m/z 918 [M−H]⁻.

### 2,5-Dioxopyrrolidin-1-yl 1-(4-((2,5-dioxo-3-(((6S)-7-oxo-6-(5-oxopyrrolidine-2-carboxamido)-7-(phenylamino)heptyl)thio)pyrrolidin-1-yl)methyl)cyclohexyl)-1-oxo-5,8,11,14,17,20-hexaoxa-2-azadocosan-22-oate (6), ST8191AA1

Compound [26] (65 mg, 0.07 mmol) in dry CH₂Cl₂ (5 mL) is added at room temperature to a solution of dicyclohexyl-carbodiimide (0.1 mmol) and N-hydroxysuccinimide (0.07 mmol), and the mixture stirred at room temperature for 3 hours. The white solid is filtered with dichloromethane to remove the dicyclohexylurea, the organic phase is washed with HCl 0.1N and H₂O, then dried over dry sodium sulfate and the solvent removed under reduced pressure. The resulting residue is purified by flash column chromatography to give the activated acid as a white solid 61 mg (85%). MS: m/z 1020 [M+H]⁺.

### Example 7: Synthesis and Characterization of Payload (9), ST8217A1

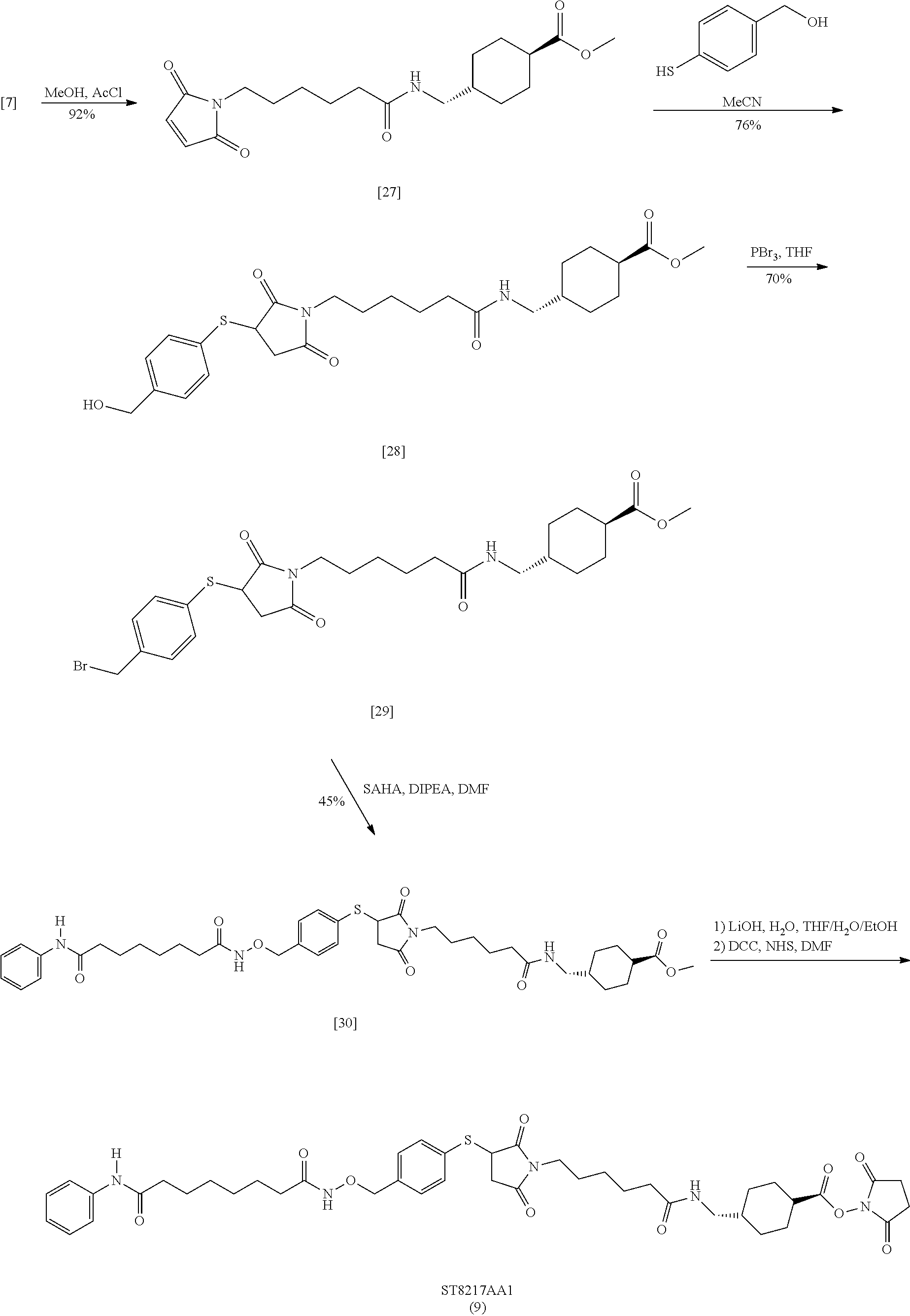

### Methyl (1R,4R)-4-((6-(2,5-dioxo-2,5-dihydro-1H-pyrrol-1-yl)hexanamido)methyl)cyclohexane-1-carboxylate [27]

Acetyl chloride (1.01 mL, 14.26 mmol) is added to a solution of acid [7] (1.00 g, 2.85 mmol) in MeOH (50 mL) in a round bottom flask under N₂. The resulting solution is stirred at room temperature for 3 h, and then the solution is concentrated in vacuo. The residue is dissolved in CH₂Cl₂ (30 mL) and washed with a saturated solution of aqueous sodium bicarbonate (3×15 mL) and brine (2×15 mL). The organic phase is dried over sodium sulfate, filtered and concentrated in vacuo to provide 950 mg (2.61 mmol) of compound [27] as a white solid (yield 92%). ¹H NMR (400 MHz, CDCl₃, δ ppm, J Hz): δ 6.65 (s, 2H), 3.62 (s, 3H), 3.50-3.46 (m, 2H), 3.07 (t, J=6.4, 2H), 2.23-2.11 (m, 3H), 1.97 (dd, J=13.6, 2.8, 2H), 1.78 (dd, J=12.8, 2.0, 2H), 1.66-1.55 (m, 4H), 1.44-1.26 (m, 5H), 0.99-0.92 (m, 2H). MS: m/z 365 [M+1]⁺; 387 [M+23]⁺. TLC R_{f}: 0.75 (CH₂Cl₂:MeOH 9:1).

### Methyl (1R,4R)-4-((6-(3-((4-(hydroxymethyl)phenyl)thio)-2,5-dioxopyrrolidin-1-yl)hexanamido)methyl)cyclohexane-1-carboxylate [28]

(4-Mercaptophenyl)methanol [36] (548 mg, 3.91 mmol) is added to a solution of compound [27] (950 mg, 2.61 mmol) in CH₃CN (20 mL) in a round bottom flask under an atmosphere of N₂. The resulting solution is stirred at room temperature for 3 h, and then the solution concentrated in vacuo. The crude reaction mixture is purified by silica gel flash chromatography (petroleum ether:EtOAc 1:4) to provide 1024 mg (2.03 mmol) of compound [28] as a pale yellow viscous oil (yield 78%).

¹H NMR (400 MHz, CDCl₃, δ ppm, J Hz): δ 7.46 (d, J=7.6, 2H), 7.31 (d, J=8.0, 2H), 5.81 (bs, 1H), 4.67 (s, 2H), 3.91-3.89 (m, 1H), 3.63 (s, 3H), 3.38-3.32 (m, 2H), 3.16-3.03 (m, 4H), 2.82 (bs, 1H), 2.21-1.96 (m, 5H), 1.77 (d, J=12.8, 2H), 1.50-1.23 (m, 9H), 1.02-0.92 (m, 2H). MS: m/z 527 [M+23]⁺; 543 [M+39]⁺. TLC R_{f}: 0.3 (EtOAc).

### Methyl (1R,4R)-4-((6-(3-((4-(bromomethyl)phenyl)thio)-2,5-dioxopyrrolidin-1-yl)hexanamido)methyl)cyclohexane-1-carboxylate [29]

PBr₃ (28 μL, 0.30 mmol) is added at 0° C. to a solution of alcohol [28] (100 mg, 0.20 mmol) in THF dry (5 mL) in a round bottom flask under an atmosphere of N₂. The resulting solution is stirred at 0° C. for 2 h and then allowed to warm to room temperature; after the addition of CH₂Cl₂ (1 mL) the solution turns orange and is concentrated in vacuo. The crude reaction mixture is purified by silica gel flash chromatography (EtOAc 100%) to provide 78 mg (0.14 mmol) of compound [29] as a bright orange oil (yield 70%). ¹H NMR (400 MHz, CDCl₃, δ ppm, J Hz): δ 7.56 (d, J=8, 2H), 7.31 (d, J=8.0, 2H), 4.66 (s, 2H), 3.92-3.89 (m, 1H), 3.60 (s, 3H), 3.37-3.32 (m, 2H), 3.16-3.05 (m, 4H), 2.21-1.99 (m, 5H), 1.77 (d, J=12.4, 2H), 1.50-1.28 (m, 9H), 1.01-0.92 (m, 2H). MS: m/z 589,591 [M+23]+. TLC R_{f}: 0.6 (EtOAc).

### p-{[N-8-Anilino-8-oxooctanoyl(aminooxy)]methyl}phenylthio)-2,5-dioxo-1-pyrrolidinyl]hexanoylamino}methyl)cyclohexanecarboxylate [30]

In a vial under N₂ atmosphere, bromide [29] (78 mg, 0.14 mmol), SAHA (50 mg, 0.18 mmol) and 1 mL of DMF dry are mixed at room temperature. Freshly distilled DIPEA (45 mg, 0.36 mmol) is added dropwise and the solution is stirred at room temperature for 12 h. The solvent is then removed via rotator evaporation and high vacuum. The residue is purified by flash column chromatography with a gradient 0-20% methanol in dichloromethane to provide the product [30] as a white solid 48 mg, 45% of yield. MS: m/z 751.4 [M+H]+. 2,5-Dioxo-1-pyrrolidinyl 4-({6-[3-(p-{[N-8-anilino-8-oxooctanoyl(aminooxy)]methyl}phenylthio)-2,5-dioxo-1-pyrrolidinyl]hexanoylamino}methyl)cyclohexanecarboxylate (9), ST8217AA1 Lithium hydroxide monohydrate (8 mg, 0.18 mmol) is added to a solution of [30](48 mg, 0.063 mmol) in a mixture of tetrahydrofuran/water/ethanol 1:1:1 (6 mL). The reaction is kept at room temperature for 2 hours, then it is diluted with ethyl acetate and washed with HCl 1N. The crude (25 mg) is directly used for the next step without any purification. Dicyclocarbodiimide (11 mg, 0.05 mmol) and N-hydroxysuccinimide (6 mg, 0.045 mmol) are added at room temperature to a stirrer solution of the crude product previously obtained (25 mg, 0.034 mmol) in DMF dry (0.80 mL). The mixture is kept at room temperature for 16 hours. The white solid formed in this reaction is filtered with dichloromethane to remove the dicylohexylurea, the organic phase is washed with HCl 0.1N and water, then dried over anhydrous sodium sulfate and the solvent removed by rotatory evaporation. The resulting residue is subjected to flash column chromatography in gradient 0-2% methanol in dichloromethane to affords the activated acid ST8217AA1 as a white viscous solid; MS: m/z 856.4 [M+Na]+.

### Example 8: Synthesis and Characterization of Payload (10), ST8201AA1

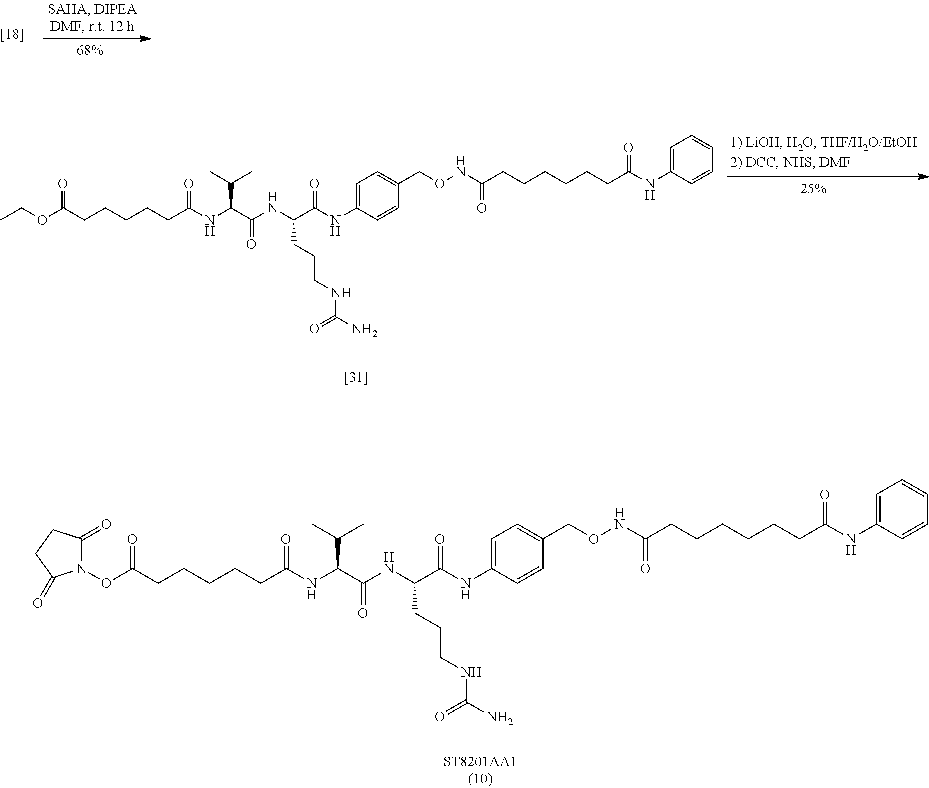 [SMILES: CCOC(=O)CCCCCC(=O)N[C@H](C(=O)N[C@@H](CCCNC(N)=O)C(=O)Nc1ccc(CONC(=O)CCCCCCC(=O)Nc2ccccc2)cc1)C(C)C ; CC(C)[C@H](NC(=O)CCCCCC(=O)ON1C(=O)CCC1=O)C(=O)N[C@@H](CCCNC(N)=O)C(=O)Nc1ccc(CONC(=O)CCCCCCC(=O)Nc2ccccc2)cc1]

### Ethyl 7-(((S)-3-methyl-1-oxo-1-(((S)-1-oxo-1-((4-(((8-oxo-8-(phenylamino)octanamido)oxy)methyl)phenyl)amino)-5-ureidopentan-2-yl)amino)butan-2-yl)amino)-7-oxoheptanoate [31]

In a vial under N₂ atmosphere, bromide [18] (60 mg, 0.09 mmol), SAHA (28 mg, 0.10 mmol) and 1 mL of DMF dry are mixed at room temperature. Freshly distilled DIPEA (25 mg, 0.20 mmol) is added dropwise and the solution stirred at room temperature for 12 h. The solvent is then removed via rotator evaporation and high vacuum. The residue is purified by flash column chromatography with a gradient 0-20% methanol in dichloromethane to provide the product [31] as a white solid 48 mg, 68% of yield. MS: m/z 795.9 [M+H]+, 818.0 [M+Na]⁺. ¹H NMR (400 MHz, DMSO) δ 7.63-7.598 (m, 2H), 7.57 (d, J=8 Hz, 2H), 7.39 (d, J=8.4 Hz, 2H), 7.313 (t, J=8 Hz, 2H), 7.09 (t, J=8; 1H), 4.89 (s, 2H), 4.58 (s, 2H), 4.20 (d, J=7.2, 1H), 4.14 (t, J=7.2 Hz, 2H), 4.10 (t, J=7.4 Hz, 1H), 3.56-3.45 (m, 1H), 3.34 (d, J=7.8 Hz, 2H), 2.41-2.37 (m, 1H), 2.34-2.30 (m, 2H), 2.13-2.06 (m, 2H), 1.79-1.60 (m, 4H), 1.42-1.36 (m, 4H), 1.28-1.24 (m, 3H), 1.01-0.97 (m, 6H).

### 2,5-Dioxopyrrolidin-1-yl 7-(((S)-3-methyl-1-oxo-1-(((S)-1-oxo-1-((4-((((S)-7-oxo-6-((R)-5-oxopyrrolidine-2-carboxamido)-7-(phenylamino)heptyl)thio)methyl)phenyl)amino)-5-ureidopentan-2-yl)amino)butan-2-yl)amino)-7-oxoheptanoate (10), ST8201AA1

Lithium hydroxide monohydrate (8 mg, 0.18 mmol) is added to a solution of 40 (48 mg, 0.06 mmol) in a mixture of tetrahydrofuran/water/ethanol 1:1:1 (6 mL). The reaction is kept at room temperature for 6 hours, then diluted with ethyl acetate and washed with HCl 1N. The crude (25 mg) is used for the next step without any purification. MS: m/z 765.7 [M−H]⁻. Dicyclocarbodiimide (11 mg, 0.05 mmol) and N-hydroxysuccinimide (6 mg, 0.045 mmol) are added at room temperature to a stirrer solution of the crude product previously obtained (25 mg, 0.03 mmol) in DMF dry (0.80 mL). The mixture is kept at room temperature for 16 hours. The white solid formed in this reaction is filtered with dichloromethane to remove the dicylohexylurea, the organic phase washed with HCl 0.1N and water, then dried over anhydrous sodium sulfate and the solvent removed by rotatory evaporation. The resulting residue is subjected to flash column chromatography in gradient 0-2% methanol in dichloromethane to give the activated acid ST8201AA1 as a white viscous solid; MS: m/z 887.2 [M+Na]⁺.

### Example 9: Synthesis and Characterization of Payload (22), ST8227AA1

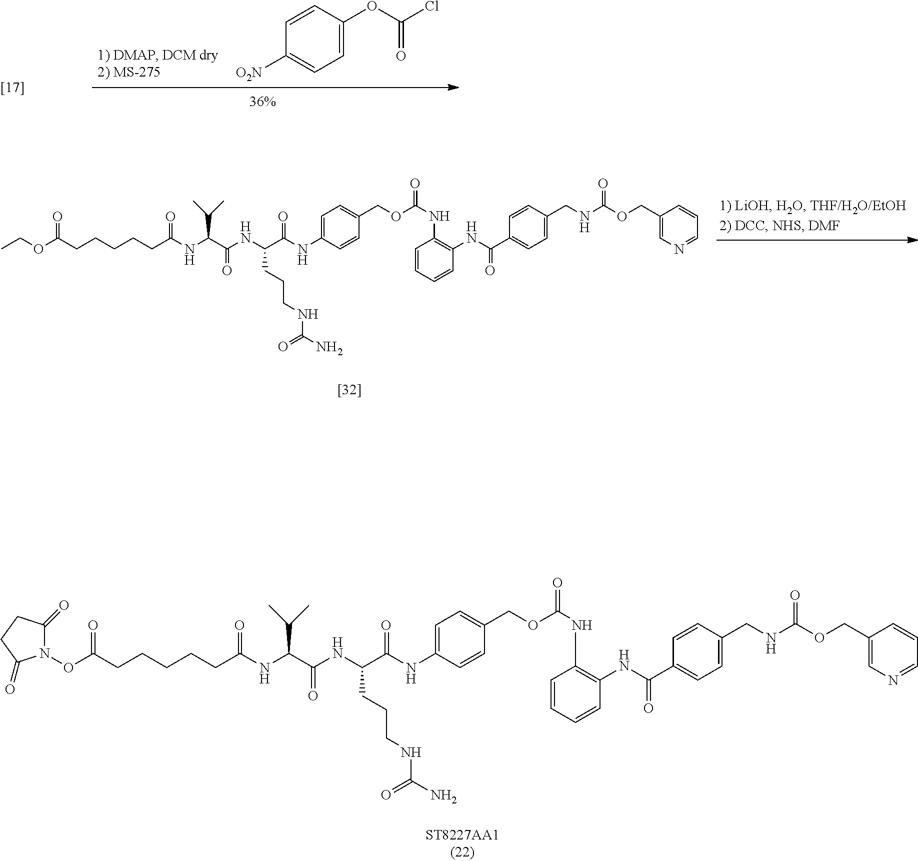

### Synthesis and Characterization of Payload (22), ST8227AA1

In a vial cooled to 0° C., under N₂ atmosphere compound [17] (50 mg, 0.09 mmol) and 4-nitrophenylchloroformate (62 mg, 0.3 mmol), 3 mL of DCM dry (with 1 drop of DMF dry) and 4-dimethylaminopyridine (60 mg, 0.49 mmol) are mixed together. Then the mixture is stirred at room temperature and monitored by TLC. When the reaction conversion of [17] is completed, MS275 (40 mg, 0.10 mmol) is added and the reaction stirred for additional 12 h at room temperature. The mixture is then diluted with dichloromethane and washed with HCl 1N, dried over anhydrous sodium sulfate and the solvent removed by rotatory evaporation. The crude ester is dissolved in a1:1:1 mixture of H₂O, THF and EtOH (5 mL) and LiOH monohydrate is added (8.2 mg, 0.2 mmol). The mixture is stirred at reflux for 2 h, then HCl added until pH 7. The solvents are evaporated under reduced pressure and the solid obtained washed with EtOH (2×10 mL). The solution is evaporated to give colourless oil. The crude ester is dissolved in a 1:1:1:1 mixture of H₂O, THF and EtOH (5 mL) and LiOH monohydrate added (8.2 mg, 0.2 mmol). The mixture is stirred at reflux for 2 h, then HCl added until pH 7. The solvents are evaporated under reduced pressure and the solid obtained washed with EtOH (2×2 mL). The residue is purified by flash column chromatography in gradient 0-20% methanol in dichloromethane to give product ST8227AA1 as a solid (12 mg, 13% yield). MS: m/z 1044.3 [M+Na]⁺.

### Example 10. Synthesis and Characterization of Payload (7), ST8197AA1

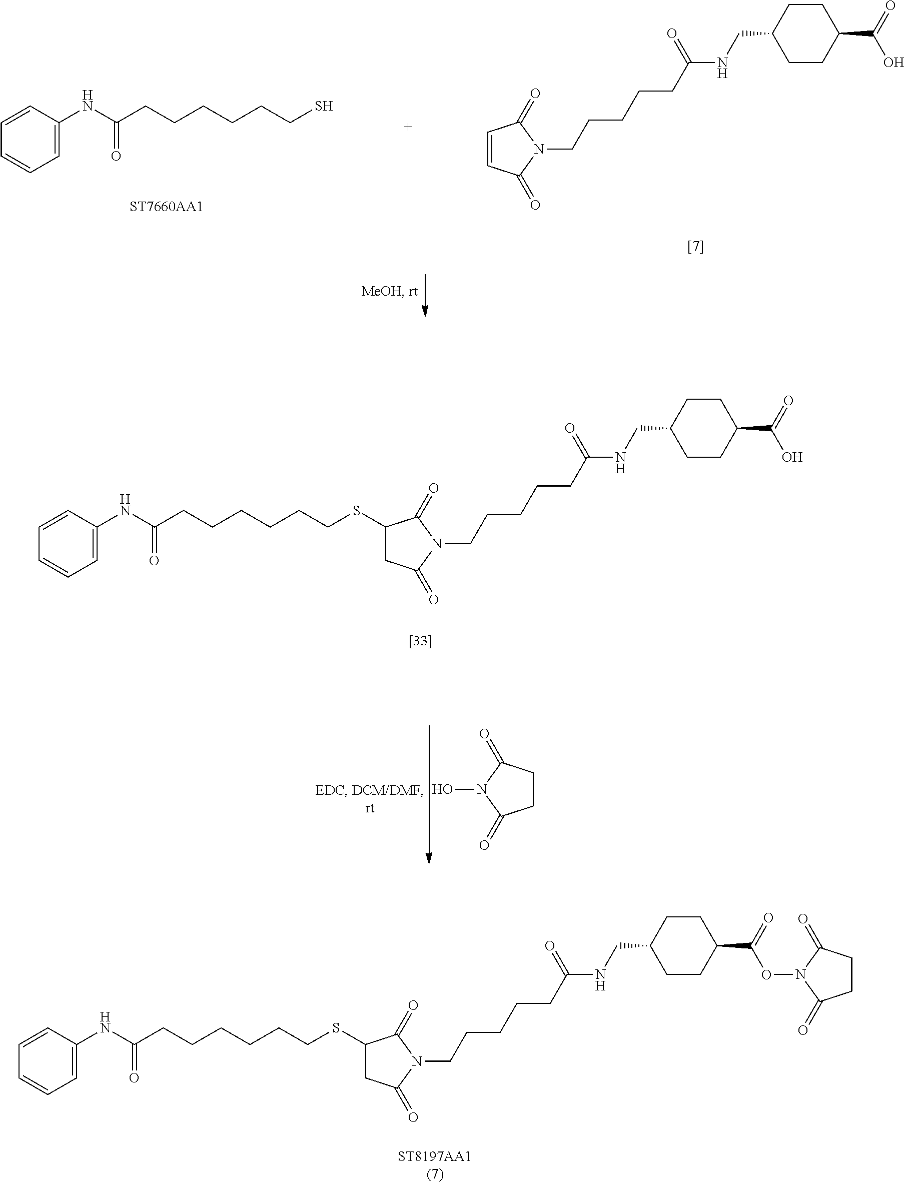 [SMILES: O=C(CCCCCCS)Nc1ccccc1 ; O=C(CCCCCN1C(=O)C=CC1=O)NC[C@H]1CC[C@H](C(=O)O)CC1 ; O=C(CCCCCN1C(=O)CC(SCCCCCCC(=O)Nc2ccccc2)C1=O)NC[C@H]1CC[C@H](C(=O)O)CC1 ; O=C1CCC(=O)N1O ; O=C(CCCCCN1C(=O)CC(SCCCCCCC(=O)Nc2ccccc2)C1=O)NC[C@H]1CC[C@H](C(=O)ON2C(=O)CCC2=O)CC1]

N-Phenyl-7-sulfanyl-heptanamide (ST7660AA1) (60 mg, 0.25 mmol) is suspended in degassed methanol (1 mL); compound [7] (84 mg, 0.24 mmol) is added at room temperature to the stirring mixture and after some minutes it becomes a clear solution. The solution is kept under stirring at room temperature for 20 hours till TLC monitoring shows complete conversion of [7]. The solvent is then removed by rotatory evaporation and the raw material is purified by column chromatography in gradient 2-10% methanol in dichloromethane. The compound [33] is obtained as a white solid, 115 mg (81% yield). MS: m/z 610 [M+Na]⁺.

¹H NMR (500 Mhz, dmso-d6) δ 11.97 (bs, 1H), 9.85 (s, 1H), 7.72 (t, J=5.6 Hz, 1H), 7.59 (d, J=7.8 Hz, 2H), 7.28 (t, J=7.8 Hz, 2H), 7.02 (t, J=7.3 Hz, 1H), 3.93 (dd, J=9.0, 3.7 Hz, 1H), 3.35 (t, J=6.8 Hz, 2H), 3.18 (dd, J=18.3, 9.0, 1H), 2.88 (t, J=6.3 Hz, 2H), 2.79-2.63 (m, 2H), 2.30 (t, J=7.3 Hz, 2H), 2.14-2.05 (m, 1H), 2.04 (t, J=7.5 Hz, 2H), 1.92-1.84 (m, 2H), 1.74-1.67 (m, 2H), 1.63-1.15 (m, 18H), 0.94-0.83 (m, 2H).

Compound [33] (110 mg, 0.19 mmol) is suspended in anhydrous dichloromethane (5.5 mL) containing 0.12 mL of dry dimethylformamide. N-hydroxysuccinimide (33 mg, 0.28 mmol) and N-(3-Dimethylaminopropyl)-N′-ethyl-carbodiimide hydrochloride (61 mg, 0.32 mmol) is added at room temperature and the reaction mixture stirred at room temperature for 24 hours. The mixture is diluted with dichloromethane (70 mL) and washed with water (40 mL) and brine (30 mL). The organic phase is dried over Na₂SO₄, filtered and concentrated by rotary evaporation. The crude is purified by flash chromatography in 2-6% methanol in dichloromethane. The product ST8197AA1 is obtained as a white solid, 97 mg (76% yield). MS: m/z 707 [M+Na]⁺.

¹H NMR (500 Mhz, dmso-d6) δ 9.83 (s, 1H), 7.73 (t, J=5.4 Hz, 1H), 7.59 (d, J=7.8 Hz, 2H), 7.28 (t, J=7.3 Hz, 2H), 7.02 (t, J=7.3 Hz, 1H), 3.94 (dd, J=8.8, 3.4 Hz, 1H), 3.36 (t, J=6.8 Hz, 2H), 3.18 (dd, J=18.3, 8.8, 1H), 2.91 (t, J=6.0 Hz, 2H), 2.81 (s, 4H), 2.80-2.62 (m, 3H), 2.30 (t, J=7.3 Hz, 2H), 2.18-1.97 (m, 4H), 1.80-1.72 (m, 2H), 1.64-1.27 (m, 16H), 1.27-1.15 (m, 2H), 1.06-0.94 (m, 2H).

¹³C NMR (500 Mhz, dmso-d6) δ 177.2, 175.6, 172.3, 171.6, 171.4, 170.7, 139.8, 129.1, 123.4, 119.5, 44.8, 39.6, 38.5, 37.1, 36.8, 36.3, 35.6, 30.8, 29.3 (4C), 29.0, 28.7, 28.6, 28.4, 27.3, 26.3, 25.9, 25.4, 25.3.

### Example 11: Synthesis and Characterization of Payloads (11) and 12, ST8215AA1 and ST8216AA1. Respectively

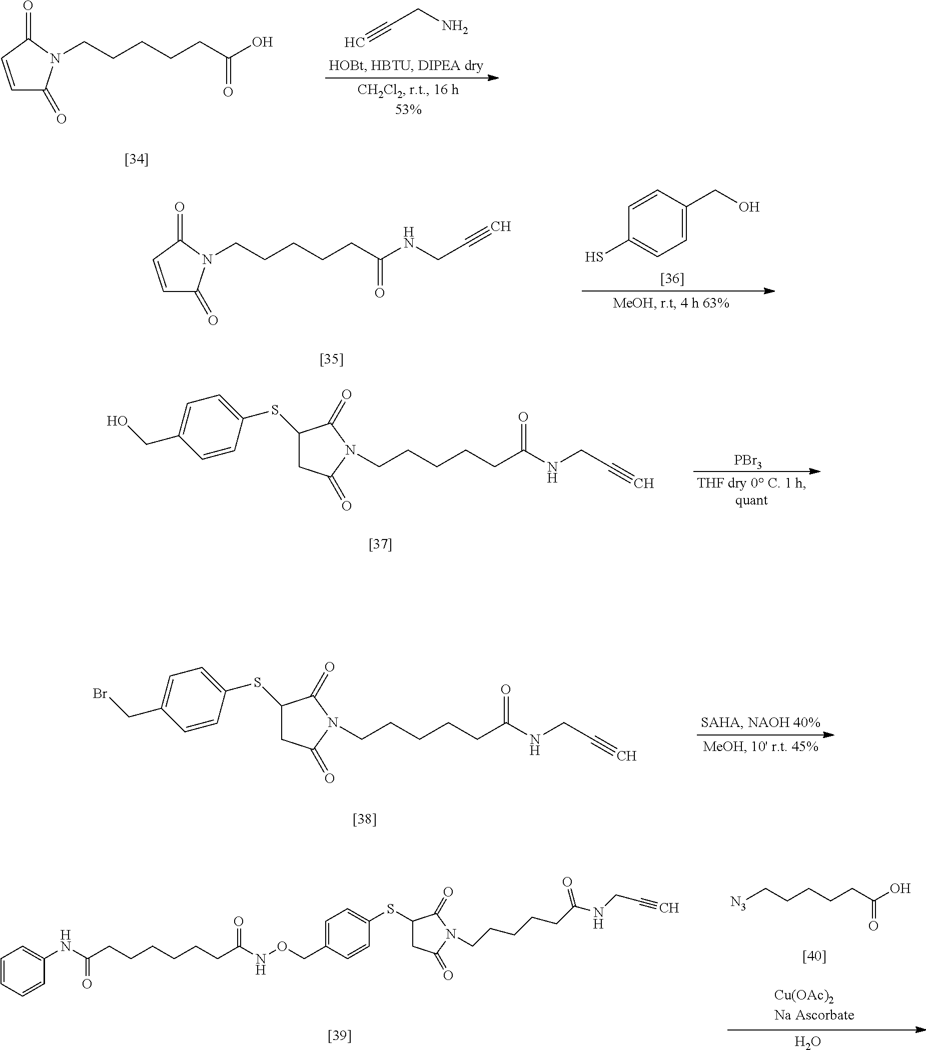

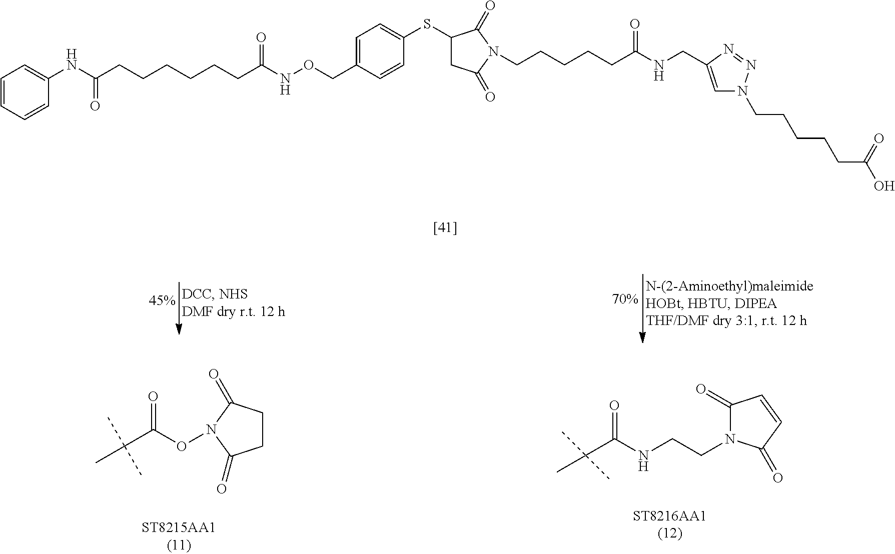

### 6-(2,5-Dioxo-2,5-dihydro-1H-pyrrol-1-yl)-N-(prop-2-yn-1-yl)hexanamide [35]

In a flask containing a solution of compound [34] (500 mg, 2.37 mmol) in dry CH₂CL₂ (25 mL), under N₂ atmosphere, propargylamine (162 μL, 2.37 mmol), HOBt H₂O (435 mg, 2.84 mmol), HBTU (1077 mg, 2.84 mmol) and DIPEA (1.24 mL, 7.10 mmol) are added at 0° C. and the mixture stirred at room temperature for 16 h. After reaction completion, the mixture is diluted with dichloromethane and washed with water, HCl 1N and brine. The organic layer is collected, dried over anhydrous sodium sulfate and the solvent removed by rotatory evaporation. The resulting residue is purified by flash column chromatography with a medium pressure system Sepacore® Buchi (silica gel; gradient petroleum ether/ethyl acetate B % 0-65 in 4 minutes, 65-65 in 7′, 65-100 in 2′) to give compound [35] (312 mg, yield 53%) as a white solid. MS: m/z 270.8 [M+Na]⁺¹H NMR (400 MHz, CDCl₃) δ 6.64 (s, 2H), 5.95 (s, 1H), 3.98 (dd, J=4, 4 Hz, 2H), 3.45 (t, J=7.2 Hz, 2H), 2.16 ((dt, J=15.0, 4.9 Hz, 2H), 1.68-1.47 (m, 4H), 1.31-1.19 (m, 2H).

### (4-Mercaptophenyl)methanol [36]

To a stirrer solution of 4-mercaptobenzoic acid (2.0 g, 13 mmol) in THF dry (25 mL) in a three-neck flask under N₂ atmosphere, LiAlH₄ 1M in THF (39 mL, 39 mmol) is added at 0° C. in 30′. The mixture is left to stir at rt for 12 hours. Then, the reaction is cooled at 0° C., quenched with 3 mL of water and acidified to pH 2 with HCl 1 M. Ethyl acetate is added and the organic layer washed with H₂O and brine, dried over anhydrous sodium sulfate, filtered and the solvent removed by rotatory evaporation. The resulting residue is purified by flash column chromatography with a medium pressure system Sepacore® Buchi (silica gel; gradient petroleum ether/ethyl acetate B % 0-25 in 3 minutes, 25-25 in 6′, 25-100 in 3′) to give alcohol [36] (1.2 mg, yield 66%) as a yellow solid. MS: m/z 141.2 [M+H]⁺¹H NMR (400 MHz, CDCl₃) δ 7.25-7.06 (m, 4H), 4.43 (s, 2H), 3.72 (s, 1H), 3.48 (s, 1H).

### 6-(3-((4-(Hydroxymethyl)phenyl)thio)-2,5-dioxopyrroidin-1-yl)-N-(prop-2-yn-1-yl)hexanamide [37]

To a stirrer solution of maleimide [35] (200 mg, 0.8 mmol) in dry MeCN (8 mL), thiol [36] (135 mg, 0.96 mmol) is added and the reaction mixture stirred under N₂ at room temperature for 4 hours. After reaction completion, the solvent is removed by rotatory evaporation and the residue purified with flash column chromatography with a medium pressure system Sepacore® Buchi (silica gel; gradient petroleum ether/ethyl acetate B % 0-80 in 5 minutes, 80-90 in 8′,90-100 in 1′) to give [37](134 mg, yield 43%) as a yellow oil. MS: m/z 410.8 [M+Na]⁺, 426.9[M+K]⁺; ¹H NMR (400 MHz, MeOD) δ 7.58 (dd, J=16.5, 8.1 Hz, 2H), 7.44 (dd, J=14.6, 8.1 Hz, 2H), 4.68 (s, 2H), 4.24 (dd, J=9.1, 3.7 Hz, 1H), 4.01 (d, J=4 Hz, 2H), 3.42 (dd, J=15.1, 7.9 Hz, 2H), 3.29 (dd, J=18.7, 9.1 Hz, 1H), 2.72 (dd, J=18.7, 3.7 Hz, 1H), 2.23 (t, J=7.5 Hz, 3H), 1.68-1.60 (m, 3H), 1.53-1.44 (m 3H), 1.31-1.16 (m 2H).

### 6-(3-((4-(Bromomethyl)phenyl)thio)-2,5-dioxopyrrolidin-1-yl)-N-(prop-2-yn-1-yl)hexanamide [38]

PBr₃ (76 μL, 0.81 mmol) is added at 0° C. to a solution of compound [37] (210 mg, 0.54 mmol) in dry THF (3 mL) and the mixture kept at 0° C. for 3 h. After evaporation of the solvent the crude is purified by flash chromatography in gradient 0-60% ethyl acetate in petroleum ether to give the product [38] (245 mg, yield >99%) as an orange solid. MS: m/z 472.8 [M+Na]⁺; 488.9 [M+K]⁺; ¹H NMR (400 MHz, CDCl₃) δ 7.49 (d, J=8.1 Hz, 2H), 7.36 (d, J=8.1 Hz, 2H), 4.46 (s, 2H), 4.04 (s, 2H), 3.58-3.34 (m, 4H), 3.16 (dd, J=18.7, 9.2 Hz, 1H), 2.69 (dd, J=18.8, 4.0 Hz, 1H), 2.30-2.12 (m, 3H), 1.74-1.58 (m, 3H), 1.56-1.43 (m, 3H), 1.27 (dd, J=16.0, 8 Hz, 2H).

### N1-((4-((2,5-Dioxo-1-(6-oxo-6-(prop-2-yn-1-ylamino)hexyl)pyrrolidin-3-yl)thio)benzyl)oxy)-N8-phenyloctanediamide [39]

SAHA (96 mg, 0.36 mmol) is dissolved in 6 mL of MeOH and NaOH 40% solution (76 μL, 0.76 mmol) is added.—After 10 min, the solution is mixed, under N₂ atmosphere, to the flask containing bromide [38] (245 mg, 0.54 mmol). Immediately the solution turns purple, and after 5-10 minutes, a TLC shows the formation of the product [39]. The methanol is rapidly concentrated, and the reaction crude diluted with dichloromethane and washed with HCl 1N to neutralize the base. The organic layer is dried over anhydrous sodium sulfate, filtered and the solvent removed by rotatory evaporation. The residue is purified by flash column chromatography in gradient 0-80% ethyl acetate in petroleum ether to provide the product [39] (90 mg, yield 40%) as a white solid. MS: m/z 656.8 [M+Na]⁺. ¹H NMR (400 MHz, CDCl₃) δ 8.16 (s, 2H), 7.50 (d, J=4 Hz, 2H), 7.41 (d, J=4 Hz, 2H), 7.33-7.23 (m, 3H), 7.03 (d, J=4 Hz, 2H), 4.84 (s, 2H), 3.95 (s, 2H), 3.40-3.31 (m, 2H), 3.10 (dd, J=20, 8 Hz, 1H), 2.64 (dd, J=18.8, 4.0 Hz, 1H), 2.29 (s, 2H), 2.19 (s, 2H), 2.11 (s, 2H), 1.64-1.57 (m, 6H), 1.29-1.22 (m, 8H), 1.10 (s, 2H), 0.83 (d, J=8.4 Hz, 1H). ¹³C NMR (100 MHz, CDCl₃) δ 175.02 (s), 174.58 (d, J=89.2 Hz), 172.58 (s), 171.55 (s), 137.86 (s), 137.84 (s), 134.37-127.29 (m), 127.98-127.29 (m), 123.66 (s), 119.52 (s), 79.30 (s), 71.03 (s), 43.28 (s), 36.62 (dd, J=207.0, 92.0 Hz), 35.71 (s), 35.59 (d, J=25.1 Hz), 35.46 (s), 29.24 (s), 28.20 (dd, J=148.7, 99.9 Hz), 26.74-26.34 (m), 26.34-24.48 (m), 24.49 (s), 24.49 (s).

### 6-Azidohexanoic Acid [40]

6-Bromohexanoic acid (200 mg, 1.02 mmol) and sodium azide (333 mg, 5.12 mmol) in 5 mL of dimethylformamide are heated at 100° C. for 16 hours in sealed vial. After cooling, the reaction is diluted with ethyl acetate and washed with KHSO₄ 1M, H₂O and brine. The organic layer is collected and dried over anhydrous sodium sulfate, and the solvent removed by rotatory evaporation. Product [40] (158 mg, 99%) is obtained pure as a brown oil, MS: m/z 155.8 [M−H]⁻; ¹H NMR (400 MHz, CDCl₃) δ 10.49 (s, 1H), 3.21-3.18 (m, 2H), 2.28 (t, J=7.4 Hz, 2H), 1.84-1.45 (m, 4H), 1.38-1.31 (m, 2H).

### 6-(4-((6-(2,5-dioxo-3-((4-(((8-oxo-8-(phenylamino)octanamido)oxy)methyl) phenyl)thio)pyrrolidin-1-yl)hexanamido)methyl)-1H-1,2,3-triazol-1-yl)hexanoic Acid [41]

A stirrer solution of compound [40] (17 mg, 0.11 mmol) and compound [39] (90 mg, 0.14 mmol) in dry DMF (11 mL) is degassed at room temperature by argon/vacuum cycles (3×). To this solution, a freshly prepared aqueous mixture (5.5 mL) of Cu(OAc)₂ (7 mg, 0.03 mmol) and sodium ascorbate (13 mg, 0.07 mmol), previously degassed by argon/vacuum cycles, is added. The reaction mixture is degassed again and left to stir under Argon at room temperature for 72 hours. After reaction completion, the crude is concentrated, and the solvent removed by rotatory evaporation. The resulting residue is subjected to flash column chromatography in gradient 0-16% methanol in dichloromethane to provide the triazole [441] (55 mg, yield 62%) as an orange solid; MS: m/z 813.8 [M+Na]⁺¹H NMR (400 MHz, MeOD) δ 7.86 (s, 2H), 7.59-7.46 (m, 4H), 7.37 (d, J=7.2 Hz, 2H), 7.26 (d, J=7.2 Hz, 3H), 7.04 (d, J=7.1 Hz, 1H), 4.37 (s, 2H), 4.21 (s, 2H), 3.33 (dd, J=12.9, 4.9 Hz, 4H), 3.18 (dd, J=20, 8 Hz, 1H), 2.60 (dd, J=18.8, 4.0 Hz, 1H), 2.42-2.12 (m, 5H), 2.02 (s, 2H), 1.88 (s, 2H), 1.76-1.47 (m, 8H), 1.49-1.23 (m, 10H), 1.17 (s. 2H), 0.86 (s, 1H). ¹³C NMR (100 MHz, MeOD) δ 140.29-139.14 (m), 139.14-134.26 (m), 132.86 (s), 139.14-108.94 (m), 107.34 (s), 76.31 (s), 35.28 (s), 28.04 (s), 25.16 (d, J=61.4 Hz), 24.57 (s).

### 2,5-Dioxopyrrolidin-1-yl 6-(4-((6-(2,5-dioxo-3-((4-(((8-oxo-8-(phenylamino) octanamido)oxy)methyl)phenyl)thio)pyrrolidin-1-yl)hexanamido)methyl)-1H-1,2,3-triazol-1-yl)hexanoate (11), ST8215AA1

Dicyclohexylcarbodiimide (21 mg, 0.10 mmol) and N-hydroxysuccinimide (10 mg, 0.09 mmol) are added at room temperature to a stirrer solution of compound [41] (55 mg, 0.06 mmol) in anhydrous dimethylformamide (0.80 mL). The mixture is kept at room temperature for 16 hours. The white solid formed in this reaction is filtrated with dichloromethane and the organic phase washed with water, then dried over anhydrous sodium sulfate, filtered and the solvent removed by rotatory evaporation. The resulting residue is subjected to flash column chromatography in gradient 0-8% methanol in dichloromethane to afford the activated acid ST8215AA1 as a white viscous solid (24 mg, 45%); MS: m/z 910.8 [M+Na]⁺; ¹H NMR (400 MHz, CDCl₃) δ 8.07 (s, 2H), 7.52 (s, 2H), 7.44 (s, 2H), 7.32 (s, 2H), 7.23 (s, 3H), 7.04 (s, 1H), 4.87 (s, 2H), 4.45 (s, 2H), 4.31 (s, 2H), 3.32 (s, 2H), 3.13 (s, 1H), 2.79 (s, 4H), 2.65 (s, 2H), 2.55 (s, 2H), 2.29 (s, 2H), 2.12 (s, 2H), 1.89 (s, 2H), 1.78-1.48 (m, 8H), 1.47-1.12 (m, 10H), 1.07 (s, 2H), 0.83 (s, 1H). ¹³C NMR (100 MHz, CDCl₃) δ 134.50 (s), 129.24 (s), 128.49 (s), 124.47-123.71 (m), 121.49 (d, J=420.4 Hz), 49.89 (s), 43.30 (s), 38.48 (s), 36.87 (s), 35.65 (s), 34.39 (s), 32.54 (s), 30.25 (s), 29.25 (s), 28.04 (s), 26.23 (d, J=107.2 Hz), 24.84 (s), 25.38-23.33 (m), 24.55 (s), 24.96-23.33 (m), 23.47 (s).

### N1-((4-((1-(6-(((1-(6-((2-(2,5-Dioxo-2,5-dihydro-1H-pyrrol-1-yl)ethyl)amino)-6-oxohexyl)-1H-1,2,3-triazol-4-yl)methyl)amino)-6-oxohexyl)-2,5-dioxopyrrolidin-3-yl)thio)benzyl)oxy)-N8-phenyloctanediamide (12), ST8216AA1

To a stirrer solution of [41] (15 mg, 0.02 mmol) in a mixture of dry THF/DMF 3:1 (1.33 mL) under N₂ atmosphere, N-(2-aminoethyl)maleimide (3.3 mg, 0.02 mmol), HOBt H₂O (4 mg, 0.03 mmol), HBTU (11 mg, 0.03 mmol) and DIPEA (7 μL, 0.05 mmol) are added at 0° C. After the addition, the mixture is kept at room temperature for 12 hours, then diluted with dichloromethane and washed with water. The organic phase is dried over anhydrous sodium sulfate, filtered and the solvent removed by rotatory evaporation. The resulting residue is subjected to flash column chromatography in gradient 0-8% methanol in dichloromethane to give product ST8216AA1 (13 mg, yield 72%) as a white viscous solid. MS: m/z 936.6 [M+Na]⁺; ¹H NMR (400 MHz, MeOD) δ 7.86 (s, 3H), 7.60-7.42 (m, 5H), 7.38-7.35 (m, 2H), 7.25 (t, J=7.9 Hz, 2H), 7.03 (t, J=7.4 Hz, 1H), 6.77 (s, 2H), 4.86 (s, 2H), 4.35 (dd, J=15.7, 8.7 Hz, 4H), 3.69 (t, J=4 Hz, 2H), 3.56 (t, J=4 Hz, 2H), 3.43-3.28 (m, 5H), 3.24-3.13 (m, 2H), 2.32 (t, J=8 Hz, 2H), 2.15 (dd, J=14.9, 7.5 Hz, 2H), 2.04 (dd, J=13.3, 6.0 Hz, 2H), 1.92-1.77 (m, 2H), 1.77-1.50 (m, 8H), 1.50-1.12 (m 10H), 0.86 (s, 1H).

### Example 12: A General Synthetic Procedure for Panobinostat- and Dacinostat-Based Payloads

Synthesis and Characterization of Dacinostat-Based Payload (15), ST8233AA1

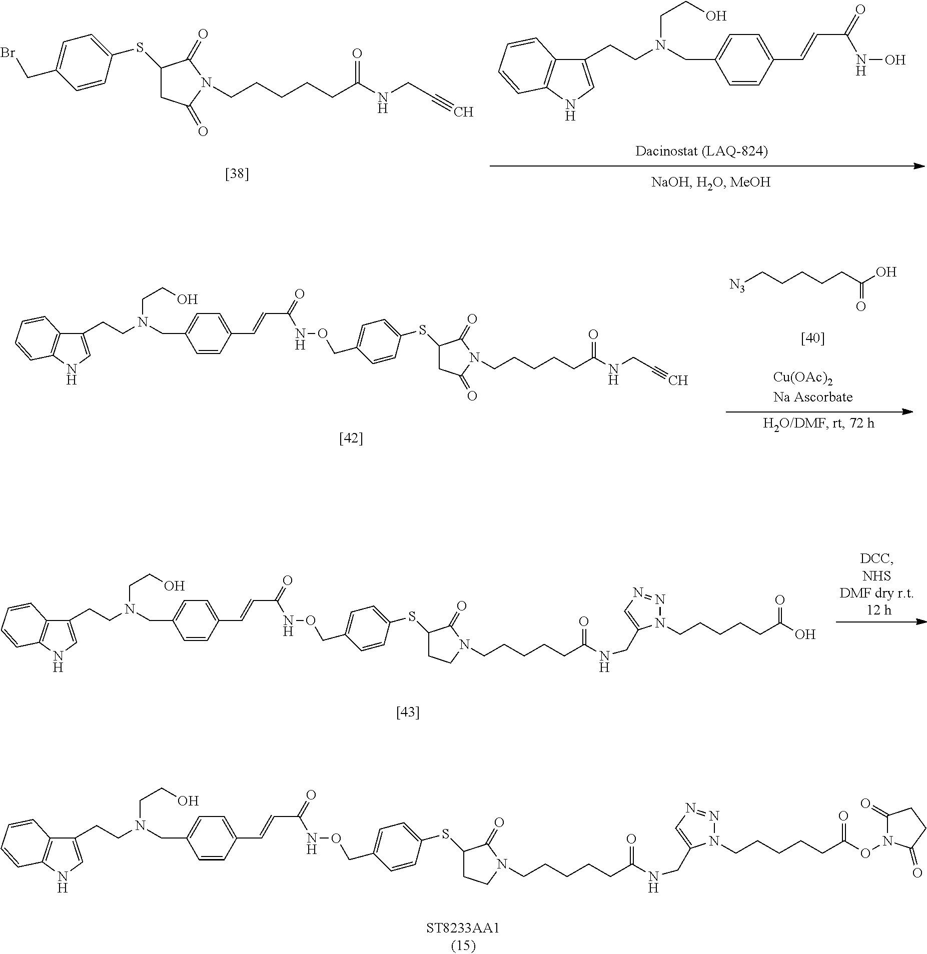 [SMILES: C#CCNC(=O)CCCCCN1C(=O)CC(Sc2ccc(CBr)cc2)C1=O ; O=C(/C=C/c1ccc(CN(CCO)CCc2c[nH]c3ccccc23)cc1)NO ; C#CCNC(=O)CCCCCN1C(=O)CC(Sc2ccc(CONC(=O)/C=C/c3ccc(CN(CCO)CCc4c[nH]c5ccccc45)cc3)cc2)C1=O ; [N-]=[N+]=NCCCCCC(=O)O ; O=C(O)CCCCCn1nncc1CNC(=O)CCCCCN1CCC(Sc2ccc(CONC(=O)/C=C/c3ccc(CN(CCO)CCc4c[nH]c5ccccc45)cc3)cc2)C1=O ; O=C(/C=C/c1ccc(CN(CCO)CCc2c[nH]c3ccccc23)cc1)NOCc1ccc(SC2CCN(CCCCCC(=O)NCc3cnnn3CCCCCC(=O)ON3C(=O)CCC3=O)C2=O)cc1]

### 3-(p-{[N-(E)-3-[p-({(2-Hydroxyethyl)[2-(1H-indol-3-yl)ethyl]amino}methyl) phenyl]acryloyl(aminooxy)]methyl}phenylthio)-1-[6-oxo-6-(2-propynylamino) hexyl]-2,5-pyrrolidinedione [42]

Dacinostat (100 mg, 0.29 mmol) is dissolved in 6 mL of MeOH containing NaOH (40% in water, 40 μL, 0.4 mmol). After stirring 15 min at rt, the solution is added to a vial containing bromide [38] (181 mg, 0.40 mmol). After 5-10 minutes of stirring at rt, a TLC analysis showed the formation of the product. Methanol is rapidly evaporated, and the reaction crude diluted with dichloromethane and washed with HCl 1N to neutralize the base, dried over anhydrous sodium sulfate, filtered and the solvent removed by rotatory evaporation. The residue is purified by flash column chromatography in gradient 0-80% ethyl acetate in petroleum ether to provide the product [42] (95 mg, yield 44%) as a white solid. MS: m/z 772.6 [M+Na]⁺. ¹H NMR (400 MHz, CDCl₃) δ, 7.83-7.72 (m, 2H), 7.65-7.30 (m, 10H), 7.25-7.07 (m, 2H), 7.13-7.05 (m, 1H), 6.81 (m, 1H), 6.64 (t-like, 1H), 4.80 (m, 1H), 4.69-4.54 (m, 2H), 4.34-4.18 (m, 2H), 4.05 (m, 1H), 3.91-3.45 (m, 7H), 3.32-2.77 (m, 8H), 2.66 (m, 1H), 2.36-2.12 (m, 4H), 1.76-1.26 (m, 8H).

### 6-[4-({6-[3-(p-{[N-(E)-3-[p-({(2-Hydroxyethyl)[2-(1H-indol-3-yl)ethyl]amino}methyl)phenyl]acryloyl(aminooxy)]methyl}phenylthio)-2,5-dioxo-1-pyrrolidinyl]hexanoylamino}methyl)-1H-1,2,3-triazol-1-yl]hexanoic acid [43]

A stirrer solution of compound [42] (40 mg, 0.05 mmol) and 6-azidohexanoic acid (16 mg, 0.10 mmol) in dry DMF (10 mL) is degassed at room temperature by argon/vacuum cycles (3×). To this solution a freshly prepared aqueous mixture (5.5 mL) of Cu(OAc)₂ (7 mg, 0.03 mmol) and sodium ascorbate (13 mg, 0.07 mmol), previously degassed by argon/vacuum cycles (3×) is added. The reaction mixture is degassed again and left to stir under Argon at room temperature for 72 hours. After reaction completion the crude is concentrated, and the solvent removed by rotatory evaporation. The resulting residue is purified by flash column chromatography in gradient 0-16% methanol in dichloromethane to provide the triazole [43](35 mg, yield 77%) as a solid. MS: m/z 930.7 [M+Na]⁺.

### Synthesis of payload (15), ST8233AA1

Dicyclohexylcarbodiimide (16 mg, 0.08 mmol) and N-hydroxysuccinimide (7.7 mg, 0.07 mmol) are added at room temperature to a stirrer solution of compound [43] (35 mg, 0.04 mmol) in anhydrous DMF (0.80 mL). The mixture is kept at room temperature for 16 h. The white solid formed in this reaction is filtrated with dichloromethane and the organic phase washed with water, then dried over anhydrous sodium sulfate, filtered and the solvent removed by rotatory evaporation. The resulting residue is subjected to flash column chromatography in gradient 0-8% methanol in dichloromethane to give the activated acid ST8233AA1 as a white viscous solid (14 mg, 35%); MS: m/z 1026.4 [M+Na]⁺.

¹H NMR (400 MHz, DMSO-d₆) δ 9.94 (bs, 1H), 8.92 (bs, 1H), 7.90 (bs, 1H), 7.61-7.47 (m, 1H), 7.50-7.37 (m, 1H), 7.33 (m, 2H), 7.20 (m, 1H), 7.15-6.86 (m, 1H), 6.69 (m, 1H), 4.80 (m, 1H), 4.69-4.54 (m, 1H), 4.49-4.12 (m, 2H), 3.84 (m 1H), 3.69-3.52 (m, 1H), 3.43-2.87 (m, 3H), 2.88-2.35 (m, 3H), 2.35-2.08 (m, 1H), 2.02-1.23 (m, 5H).

### Example 13: Synthesis and Characterization of ADCs by Conjugation to ε-Amino Groups of Lysine Residues to N-Hydroxysuccinimide (NHS)

Here, by way of example, ADCs made of HDAC is conjugated to four different antibodies (Trastuzumab Herceptin® Roche; Cetuximab Erbitux® Merck; Bevacizumab Avastin® Genentech/Roche; Panitumumab Vectibix® Amgen) and two mouse anti-human CD4 antibodies commercially available, the clone SK3 (also known as Leu3a) and the clone RPA-T4. Specifically, the approved antibodies are Trastuzumab recognizing ErbB2, Cetuximab and Panitumumab recognizing ErbB1, Bevacizumab recognizing VEGFR.

Antibodies were buffer exchanged using a 10 kDa cut-off dialysis membrane to yield antibodies solution in PBS pH 7.4 and to remove interfering preservative. The concentration after dialysis was determined measuring the OD₂₈₀ and—the absorbance reading of the sample—was divided by 1.36.

A 10 mM of a stock solution contains an amine-reactive N-hydroxysuccinimide (NHS ester) payload was prepared in DMSO and a 20-fold molar excess of payload was added to each one of antibody solution.

Reactions were incubated at room temperature, with gentle continuous mixing and after 1 hour, they were quenched adding a 20 mM glycine aqueous solution.

The final products were then dialyzed in PBS overnight at 4° C. using a 10 kDa cut-off membrane in order to remove the excess of unreacted payload.

DARs (Drug-Antibody Ratio) were determined by MALDI mass spectrometry, using an Ultraflex III mass spectrometer (Bruker, GmbH), operating in positive linear mode.

Briefly, 100 μl of unconjugated antibodies and of obtained ADCs were desalted using PD spin trap G25 (GE Healthcare) eluting in water. A 10 mg/ml s-DHB MALDI matrix solution was prepared in 0.1% TFA dissolved in H₂O:ACN (50:50, v/v). 2 μl of samples solution (antibodies or ADCs) were deposited on MALDI target using a double layer sample deposition method. The mass spectra were acquired in a mass to charge range starting from 50 kDa to 180 kDa.

The mass difference between unconjugated and conjugated antibodies was used to determine the DAR.

The antibodies Trastuzumab, Cetuximab, Panitumumab, Bevacizumab as well as the two anti-human CD4 antibodies were used for the conjugation reactions under the experimental conditions described above. ADCs with DAR ranging from 3 to 9 were obtained.

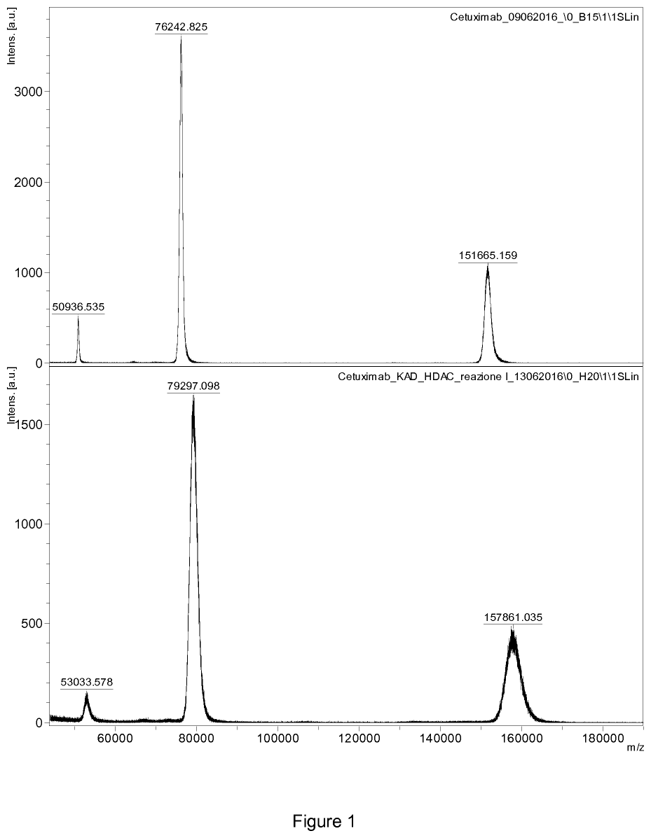

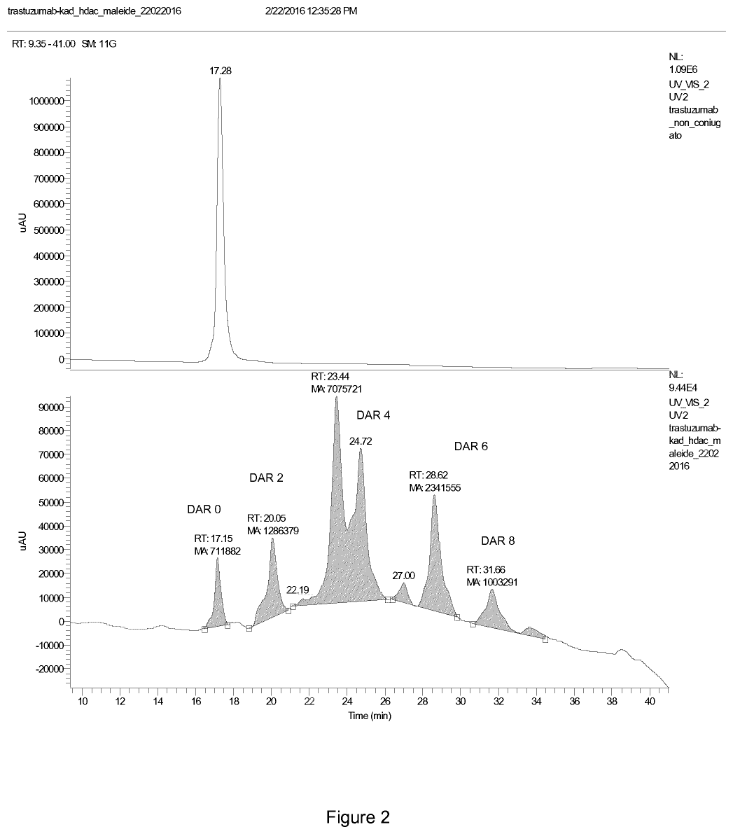

All synthesized ADCs were characterized via MALDI mass spectra. An example of lysine-based ADC and an example of cysteine-based ADC were reported in FIG. 1-2.

Here, just as example, the MALDI mass spectra of an ADC—(24), ST8154AA1—is reported (FIG. 1).

### Example 14: Synthesis and Characterization of ADCs Prepared by Conjugation at the Cysteine Residue with Maleimide-Containing Payloads

Antibodies were buffer exchanged using a 10 kDa cut-off dialysis membrane to yield antibodies solution in PBS pH 7.4 and to remove interfering preservative. The concentration after dialysis was determined measuring the OD₂₈₀ and the absorbance reading of the sample was divided by 1.36.

Mild reduction of interchain disulfides in antibodies yields unpaired cysteines in reduced form, suitable for conjugation with conventional maleimide linkers to afford homogeneous ADCs with 2-6 drugs/antibody.

For this purpose, 1 mM TCEP-HCl stock solution was prepared in water and 5-fold molar excess was added to the antibody solutions. The reactions were maintained at 37° C. for 2 hours with gentle continuous mixing. The reactions was cooled.

Since TCEP is a thiol-free compound, removing the excess of the reducing was not necessary and a 20-fold molar excess of a 10 mM maleimide-based payload stock solution prepared in DMSO, was added to each one of reduced antibody solutions.

Reactions were incubated at room temperature, with gentle continuous mixing and after 1 hour they were quenched adding a 20 mM cysteine aqueous solution.

The final products were then dialyzed extensively in PBS overnight at 4° C. using a 10 kDa cut-off membrane in order to remove the excess of unreacted payload.

DARs (Drug-Antibody Ratio) were determined by hydrophobic chromatography, using an MabPac HIC-Butyl column 100 mm×4.6 mm, 5 μm (Thermo Fisher Scientific). The mobile phase A was 1.5 ammonium sulfate, 50 mM sodium phosphate, pH 7 and isopropanol (95:5; v/v), and the mobile phase B was 50 mM sodium phosphate, pH 7 and isopropanol (80:20; v/v).

The mobile phase A was maintained at 100% for 1 minute after the injection and then the mobile phase B was increased to 100% in 30 minutes and hold for 5 minutes.

The UV profiles were registered at 220 and 280 nm. All antibodies, such as Trastuzumab and Cetuximab, according to the present invention, were prepared by a process which involve conjugation reactions under the experimental conditions described above. ADCs with an average DAR ranging from 3.5 to 4.6 were obtained.

Here, just as example, HIC chromatogram of an ADC—(28), ST8176AA1—is reported (FIG. 2). The DAR were calculated considering the peak areas and applying the following formula:

$$DAR = \frac{{\sum }_{0}^{B} nAreaDARn }{{\sum }_{0}^{B} AreaDARn}$$

### Example 15: Determination of ADCs Binding to ErbB1 and ErbB2 Receptors on Tumor Cells

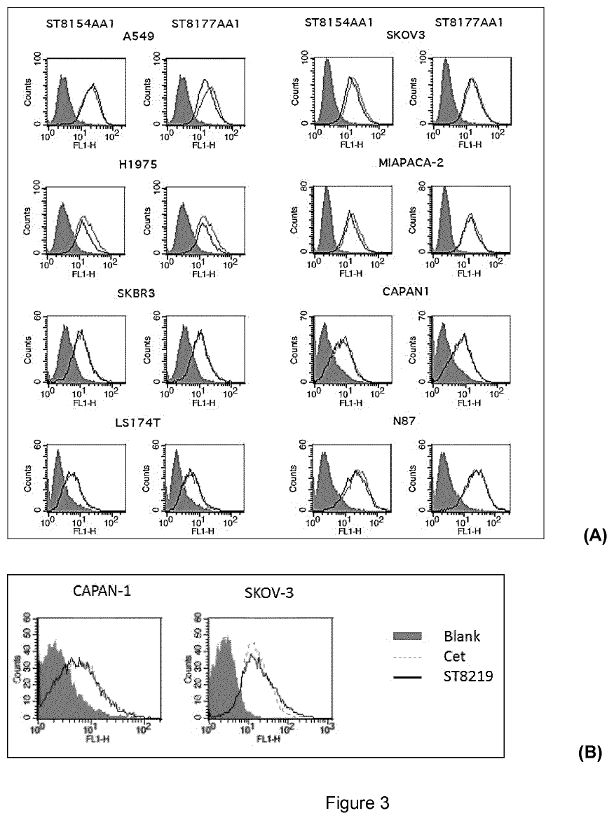

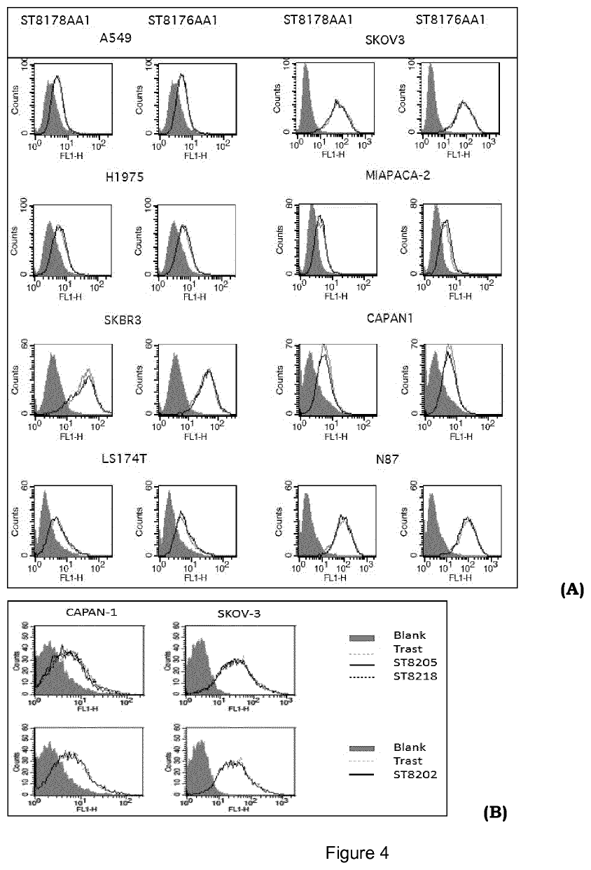

Binding of the ADCs to ErbB1- and ErbB2-expressing tumor cells was confirmed by FACS analysis. Various tumor cell lines with different levels of EGFR and ErbB2 expression, including lung (A549, H1975), breast (SKBR3), colon (LS174T), ovarian (SKOV3), pancreas (CAPAN1 and MIAPACA-2) and stomach (N87) carcinoma cell lines, were used in the experiments. Cell pellets were incubated 1 hour, at 4° C., with 10 μg/mL antibodies or ADCs in PBS and then, after two washings in PBS, stained 1 hour, at 4° C., with mouse anti-human FITC-conjugated IgGs (BD Pharmingen). After two further washings, propidium iodide (PI) was added and cell-associated fluorescence analysis was performed by means of a FACScalibur (Becton Dickinson). All the compounds showed to bind to receptors in a comparable manner to that of the free antibodies, Cetuximab and Trastuzumab (see Table 3 and FIGS. 3 and 4).

TABLE 3
ADC binding to ErbB1 and ErbB2 receptors and internalization

| | | | | | | Binding | |
|---|---|---|---|---|---|---|---|
| | | | | | | (FACS | Internalization |
| I.D. | Code number | Drug | Payload | mAb | DAR | analysis) | (HCS Imaging) |
| (24) | ST8154AA1 | ST7464AA1 | ST8128AA1 | Cet | 5.0-9.0 | ++ | ++ |
| (25) | ST8155AA1 | ST7464AA1 | ST8132AA1 | Cet | 6.1-6.2 | ++ | ++ |
| (26) | ST8177AA1 | ST7464AA1 | ST8152AA1 | Cet | 3.2-4.6 | ++ | ++ |
| (27) | ST8178AA1 | ST7464AA1 | ST8128AA1 | Tras | 7.9 | ++ | ++ |
| (28) | ST8176AA1 | ST7464AA1 | ST8152AA1 | Tras | 4.1-4.5 | ++ | ++ |
| (29) | ST8179AA1 | ST7464AA1 | ST8132AA1 | Tras | 6.3 | ++ | ++ |
| (30) | ST8205AA1 | ST7660AA1 | ST8197AA1 | Tras | 6.2 | ++ | ++ |
| (36) | ST8218AA1 | Vorinostat | ST8217AA1 | Tras | 5.0 | ++ | ++ |
| (37) | ST8219AA1 | Vorinostat | ST8217AA1 | Cet | 4.0 | ++ | ++ |
| | | | | Cet | | ++ | ++ |
| | | | | Tras | | ++ | ++ |

++ is a score representing a high extent of binding or internalization.
Cet = Cetuximab; Tras = Trastuzumab

### 16: Determination of Internalization by Tumour Cells

Ability of ADCs to internalize within tumour cells, following their binding to cognate receptors, was assessed by means of HCS fluorescence imaging, through Operetta system (Perkin Elmer). Several tumour cell types, including lung (A549, H1975), breast (SKBR3), colon (LS174T), ovarian (SKOV3), pancreas (CAPAN1) and stomach (N87) carcinoma cell lines, were tested. Cells were seeded in 96-well microtiter plates (0.5-1×10⁴ well) and then incubated with 5 μg/mL antibodies in culture medium, for 3 hours at 37° C. Following two washings, cells were then fixed with 4% formaldehyde in PBS, permeabilized with PBS 0.2% Tween-20 (PBS-T) and blocked with 2% BSA in PBS-T, and ultimately stained with FITC-conjugated mouse anti-human IgG (BD Pharmingen). Fluorescence was acquired and analyzed by means of the High Content Screening (HCS) system Operetta (Perkin Elmer). Cells were counterstained with Draq5 dye (Cell Signaling).

All ADCs bound to their cognate receptors and internalized by the target cells in a comparable manner to that of their native counterpart antibodies, Cetuximab and Trastuzumab (see Table 3).

### Example 17: Determination of the Binding of ADCs on Receptors by ELISA and Biacore

Immunoreactivity of ADCs was tested by antigen-specific ELISA. Briefly, Immuno MAXISORP 96-well plates (Nunc) were coated overnight at 4° C. with 50 ng/well of recombinant human EGF-R/ErbB1 Fc chimera (R&D) or recombinant Human ErbB2/HER2 protein (Sino Biological Inc.). After washing with PBS/0.1% Tween (PT) solution, plates were blocked 2 hours at room temperature (RT) with PT solution containing 1% BSA (PTB), and then incubated with serial dilutions of antibodies, 1 h at room temperature. After additional washings, anti-human K light chain horseradish peroxidase (HRP)-conjugated antibody (Sigma Aldrich), diluted 1:1,000 in blocking solution, was added 1 h at room temperature. Following four washings with PT solution, 200 μL/well TMB substrate (Sigma Aldrich) were added and plates were incubated 30 min at 37° C. The reaction was blocked by adding 100 μL/well of 0.5M H₂SO₄ solution, and optical density at 450 nm measured by ELISA spectrophotometer.

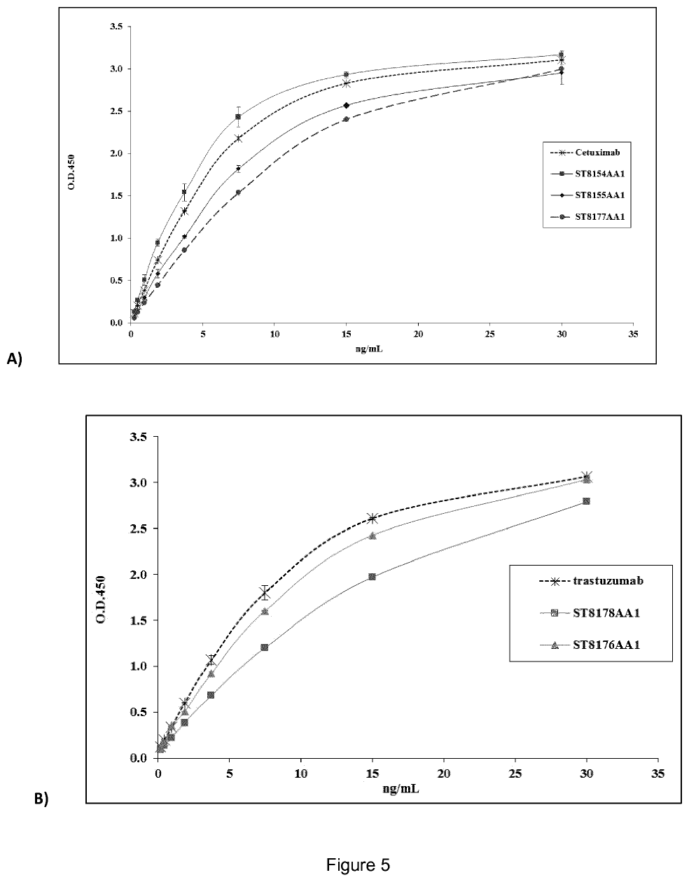

All tested ADCs showed to react with their specific receptor with potency comparable to that of native antibodies Cetuximab and Trastuzumab (FIGS. 5A and 5B).

The binding affinity of cetuximab and its corresponding ADCs, ST8155AA1 and ST8154AA1, to EGFR/ErbB1 Fc chimera protein, was measured by means of surface plasmon resonance (SPR) analysis on a Biacore T200 biosensor (GE). Briefly, EGFR/ErbB1 Fc chimera protein was coupled to a research-grade carboxymethylated dextran sensor chip (CM5, Biacore) using the amine coupling kit sup-plied by the manufacturer. Kinetic analyses were performed employing the single cycle kinetics assay, in order to avoid the extensive use of the regeneration procedure that is detrimental to the ligand (regeneration turned out to be necessary, since the antibodies did not dissociate at the end of each cycle). Antibodies and ADCs were injected at a concentration range of 6.25-100 nM in PBS running buffer at pH 7.2. The compounds were injected from low to high concentration with 180 s contact time and 600 s dissociation time in between. Injections were performed at 25° C. with a flow rate of 30 μL/min. Sensor chip surface was regenerated with 10 mM glycine/HCl pH 2.5 for 12-20 s. All the experiments were performed in duplicate. The reported kon and koff values are the average of values arising from the two experiments. KD=koff/kon (see Table 4).

TABLE 4
Biacore analysis for ST8154AA1 (24) and ST8155AA1 (25).

| Cetuximab | Kon (1/Ms) | Koff(l/s) | KD(M) |
|---|---|---|---|
| | 1.59 × 10⁵ | 4.54 × 10⁻⁴ | 2.85 × 10⁻⁹ |
| ST8154AA1 | 1.77 × 10⁵ | 4.24 × 10⁻⁴ | 2.40 × 10⁻⁹ |
| ST8155AA1 | 6.82 × 10⁴ | 3.51 × 10⁻⁴ | 5.15 × 10⁻⁹ |

### Example 18: Determination of Integrity of ADC after Nebulization Process

Nebulization has been recently shown to be a promising delivery method for mAbs in respiratory diseases, representing a non-invasive method suitable for targeting drugs directly to the lungs, limiting the exposure of secondary organs.

According to this premise, the present inventors sought to assess by HPLC analysis the recovery and integrity of ADCs following nebulization. Briefly, 300 μg/mL solutions (in PBS) of ADCs and their parental antibodies, cetuximab and trastuzumab, were nebulized for 5 minutes through a conventional jet nebuliser (AirFamily system, Pic indolor). Nebulized drugs were then collected by conveying the mist in falcon tubes and 100 μL of condensed solution was analysed by SEC-HPLC (TSKgel G3000 SWXL column, TOSOH Bioscience) in comparison to pre-nebulized samples.

Percentage of recovery after nebulization was calculated by measuring the area of each relative peak with respect to pre-nebulized samples, and ranged from 50% to 30% and from 40% to 20% for ADCs in the case of cetuximab-based and trastuzumab-based ADCs, respectively. Integrity and aggregation incidence were also assessed for each nebulized ADC, according to the profile of each chromatogram, and compared to those of not-nebulized samples (see Table 5).

TABLE 5
Characterization of ADCs, by HPLC, following nebulization

| | | | | | Recover of |
|---|---|---|---|---|---|
| | | | | | principal |
| Code number | | Integrity (%) | Aggregates (%) | Degrades (%) | peak (%) |
| Cetuximab | Pre neb | 100 | 0 | 0 | 72 |
| | neb | 100 | 0 | 0 | |
| ST8154AA1 | Pre neb | 98 | 2 | 0 | 47 |
| (24) | neb | 76 | 24 | 0 | |
| ST8155AA1 | Pre neb | 95 | 4 | 1 | 33 |
| (25) | neb | 69 | 30 | 1 | |
| ST8177AA1 | Pre neb | 84 | 1 | 15 | 30 |
| (26) | neb | 82 | 5 | 13 | |
| Trastuzumab | Pre neb | 100 | 0 | 0 | 58 |
| | neb | 100 | 0 | 0 | |
| ST8178AA1 | Pre neb | 98 | 2 | 0 | 20 |
| (27) | neb | 85 | 15 | 0 | |
| ST8176AA1 | Pre neb | 83 | 5 | 12 | 21 |
| (28) | neb | 76 | 11 | 13 | |
| ST8179AA1 | Pre neb | 97 | 3 | 0 | 27 |
| (29) | neb | 76 | 24 | 0 | |

### Example 19: Determination of the Capacity of ADCs to Inhibit Tumor Cell Proliferation

The effect of ADCs on cell proliferation was evaluated on two lung adenocarcinoma cell lines (NCI-H1975 and Calu-3). More in details, NCI-H1975 tumor cells are characterized by overexpression of double-mutant (L858R, T790M) ErbB1 gene, whereas Calu-3 express the wild type form of EGFR but mutant K-Ras (G13D) gene, as well as mutant TP53 and CDKN2A genes.

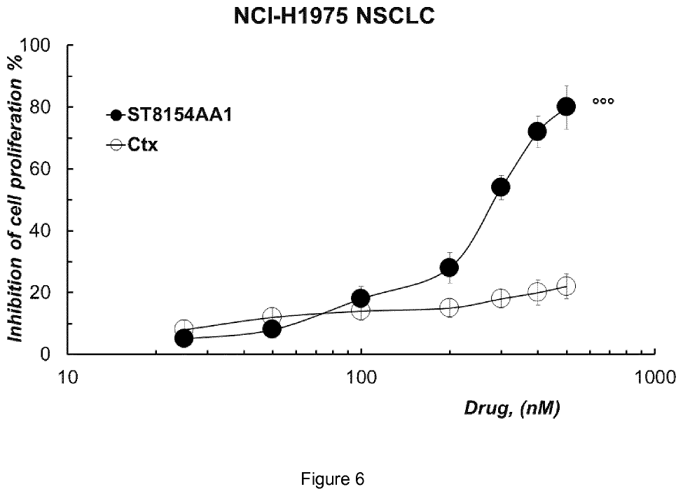

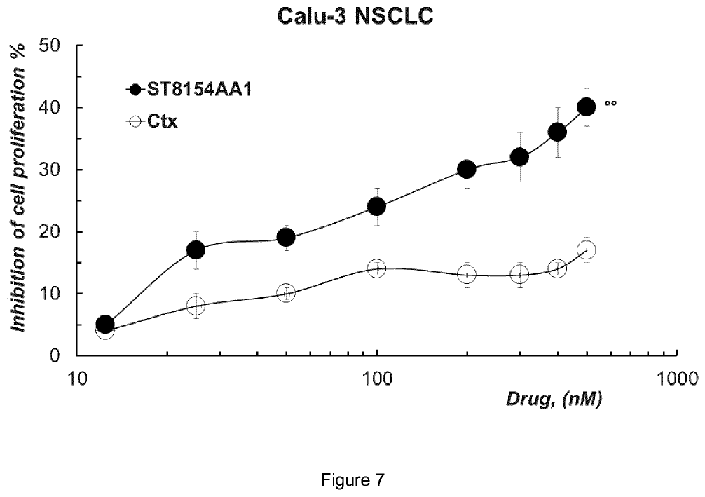

Cells were seeded (at 3.000-5.000 cells/well) into 96-well plates in complete culture medium and then incubated for 6 days, in quadruplicate, with scalar concentrations of ADCs, ranging from 500 to 6.25 nM. Inhibition of cell proliferation was measured by CellTiter-Glo Luminescent Cell Viability Assay (Promega), through a Veritas luminometer (Promega). Data were expressed as the average (±SD) of percentage inhibition of two independent experiments. The IC₅₀ values were ultimately calculated by using the GraphPad Prism 5.0 software. Results showed that ADC ST8154AA1 significantly inhibited tumor cell proliferation of both cell lines (with IC₅₀ values of 250 nM and 450 nM, on NCI-H1975 and Calu-3 cells, respectively), as compared to cetuximab alone that, instead, was not effective (IC50>500 nM) (FIGS. 6-7).

### Example 20: Determination of Activity of ADCs on Acetylation of Histone H3 and α-Tubulin in Tumor Cells

The effect of ADCs on acetylation of typical HDAC substrates, i.e. histone H3 and α-tubulin protein, was assessed by means of HCS fluorescence imaging on different tumor cells. Several tumor cell lines expressing various levels of EGFR and HER2 receptors, including lung (A549, H1975), breast (SKBR3), colon (LS174T), ovarian (SKOV3), pancreas (CAPAN1 and MIAPACA-2) and stomach (N87) carcinoma cell lines, were tested. Cells were seeded in 96-well microtiter plates (0.5-1×10⁴/well) in complete culture medium and, the day after, incubated with 5 μg/mL antibodies or ADCs, for 3 or 24 hours at 37° C. Following two washings with PBS, cells were fixed with 4% formaldehyde in PBS, permeabilized with PBS 0.2% Tween-20 (PBS-T) and blocked with 2% BSA in PBS-T. Mouse anti-acetylated-α-tubulin IgG (clone 6-11B-1, from Sigma Aldrich) or rabbit anti-acetylated-histone H3 IgG (from Active Motif) were then added in PBS-T, and cells were incubated 1 hour at room temperature. After two washings with PBS, cells were ultimately stained for 1 additional hour with FITC-conjugated goat anti-mouse or anti-rabbit IgG (BD Pharmingen), according to the primary antibody used. Fluorescence was acquired and analyzed by means of the High Content Screening (HCS) system Operetta (Perkin Elmer). Cells were counterstained with Draq5 dye (Cell Signaling).

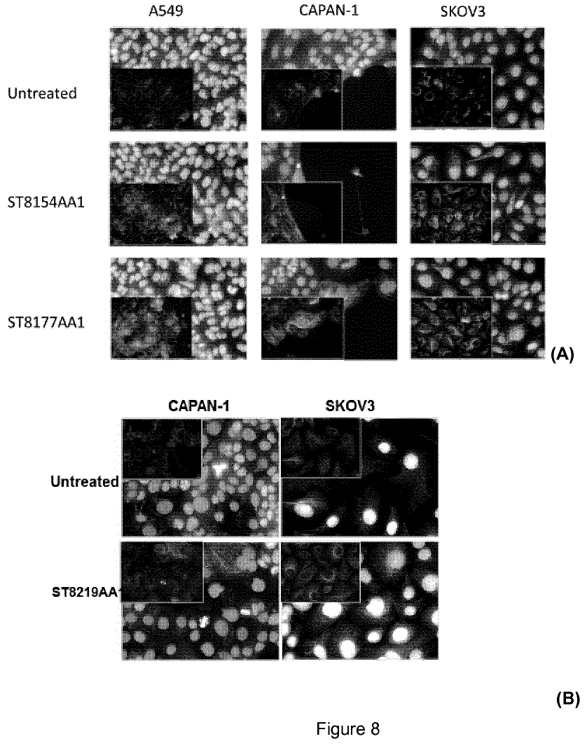

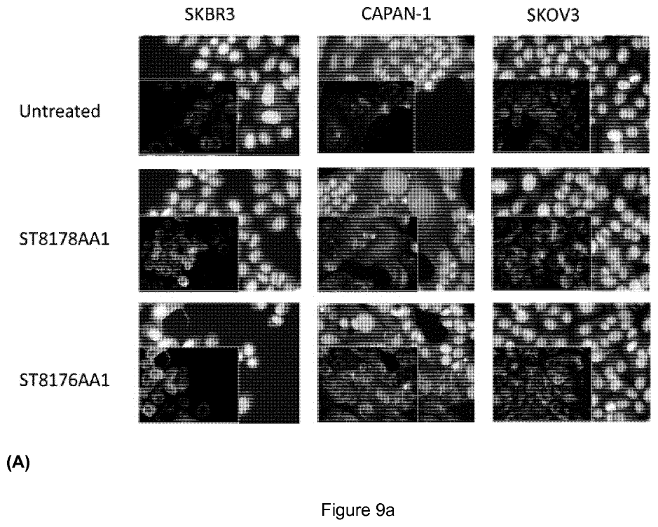

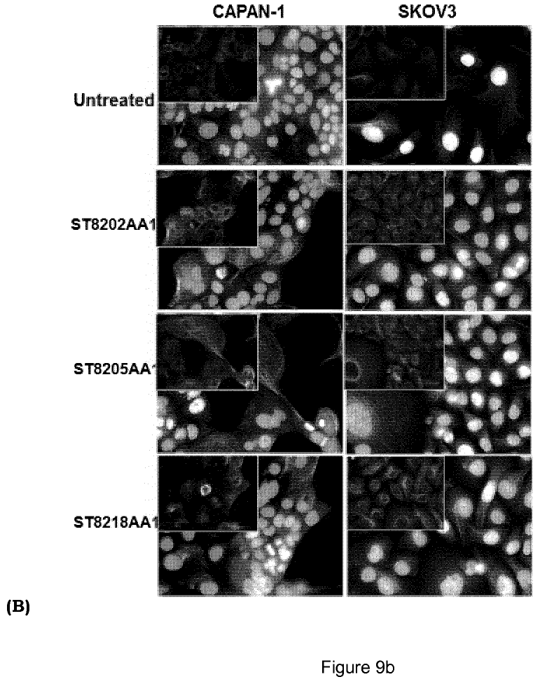

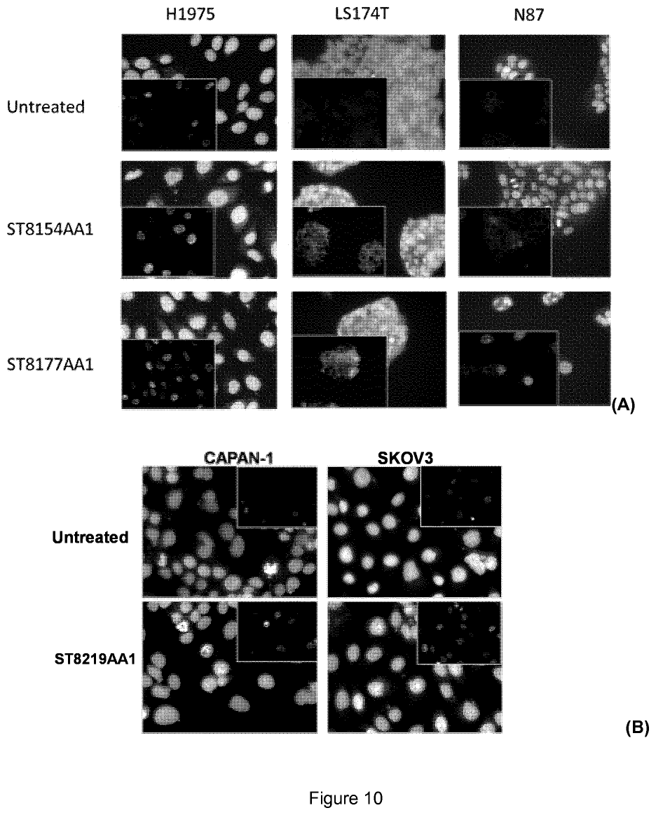

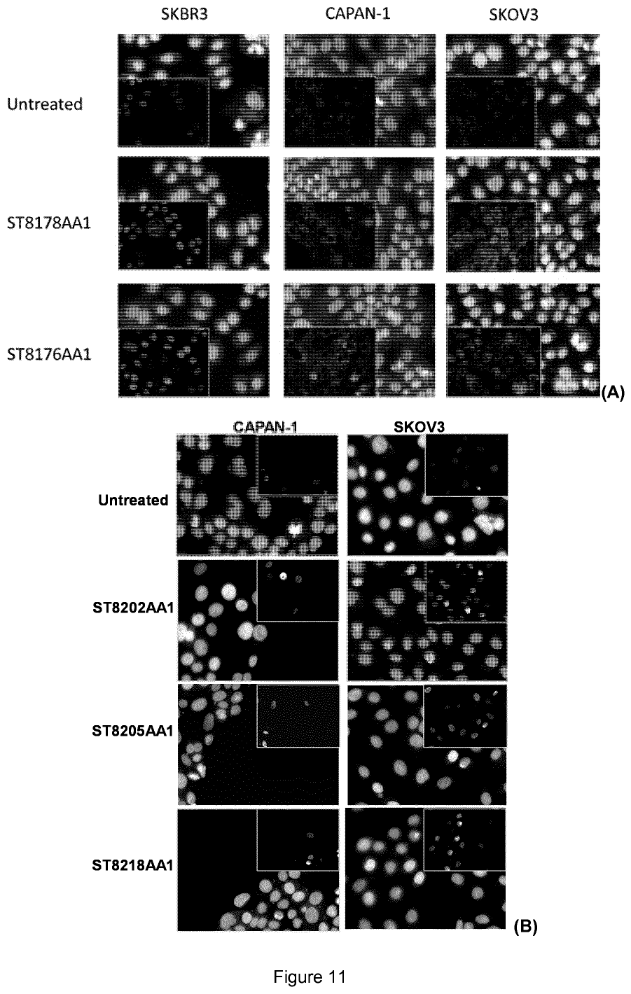

All tested ADCs induced a relevant increase in the acetylation level of both α-tubulin and histone H3 in all tested tumor cell lines, as a result of direct enzymatic inhibition of HDAC6 and class I HDACs, respectively (FIGS. 8-11). This result was coherent with the ability of ADCs to be internalized and degraded to release the ST7464AA1 moiety.

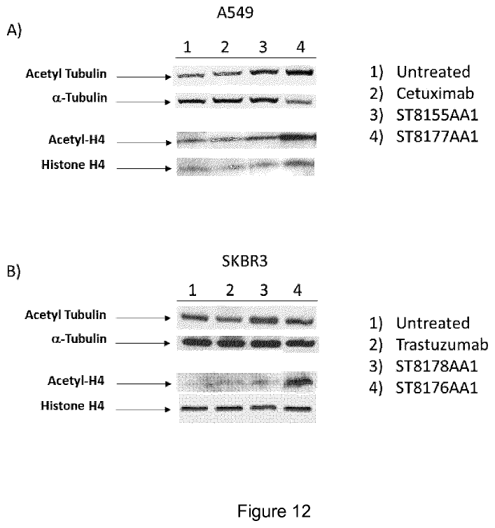

Increased acetylation of α-tubulin and histone H4 was also observed by means of Western Blot analysis on protein lysates of different tumor cells. Briefly, the day before the experiment tumor cells were seeded into 6-well dishes in complete culture medium. Cells were then treated, for 3 hours at 37° C., with 20 μg/mL test antibodies. After treatment, cells were washed twice with ice-cold PBS and then whole cell lysate was prepared by incubation, 10 min on ice, with 1× Lysis Buffer (Cell Signaling) supplemented with protease inhibitors. Cell lysates were subjected to sonication prior to centrifugation at 14.000×g, for 10 min at 4° C., to remove cell debris. The protein content was determined by the classical colorimetric Bradford method (Coomassie Bradford protein assay kit; Pierce), according to the manufacturer's instruction. To assess the extent of acetylation, equal amounts of proteins for each sample were resolved by SDS-PAGE and transferred onto a nitrocellulose membrane (Hybond-C extra, Amersham-GE Healthcare). Molecular weights were estimated based upon the relative migration with molecular weight protein markers (Prestained Kaleidoscope Standards; Bio-Rad). Nonspecific binding sites were then blocked by incubation of the membranes with 5% non-fat dry milk in TBS, overnight at 4° C. Specific primary antibodies (rabbit anti-acetyl-histone H4 antibody, from Santa Cruz; mouse anti-histone H4 monoclonal antibody, from Cell Signaling; mouse anti-acetylated α-tubulin monoclonal antibody, from Sigma Aldrich, rabbit anti-α-tubulin antibody, from abcam), were added to the membranes at the optimal dilution in 5% nonfat dry milk/TBST overnight at 4° C. Following four washes in TBST, membranes were incubated for 1 h with HRP-conjugated secondary antibodies in 5% non-fat dry milk/TBST. Immunoreactive bands were finally visualized by enhanced chemiluminescence with the ECLplus Western blotting detection reagent (GE Healthcare), and analyzed by a phosphoimaging system (STORM, Molecular Dynamics). Representative blots are shown in FIG. 12.

### Example 21: Determination of In Vivo Antitumor Effect of ST8154AA1 and ST8155AA1 Given Ip Against NCI-H1975 Non-Small Cell Lung Cancer

Nude Nu/Nu female mice (from Charles River, Italy) were given a single subcutaneous injection of 5×10⁶ NCI-H1975 non-small cell lung carcinoma cells suspended in 100 μL cell culture medium RPMI supplemented with 10% FBS. Cetuximab and ST8154AA1 and ST8155AA1 were given intraperitoneally every 4 days for 4 days (q4dx4) at a dose of 50 mg/kg, whereas ST7612AA1 was administered intraperitoneally at 120 mg/kg according to the schedule q4dx4. Vehicle group was treated by PBS. Tumor growth was measured with a caliper and tumor volume was calculated using the formula length (mm)×width 2 (mm)/2. The tumor inhibition was calculated according to the equation: % TVI=100−(mean tumor volume of treated group/mean tumor volume of control group)×100. Toxicity was evaluated on the basis of the body weight reduction.

Animals were euthanized by CO₂ inhalation when the tumors reached a volume around 1200 mm³.

All the procedures adopted for housing and handling of animals were in strict compliance with Italian and European guidelines for Laboratory Animal Welfare.

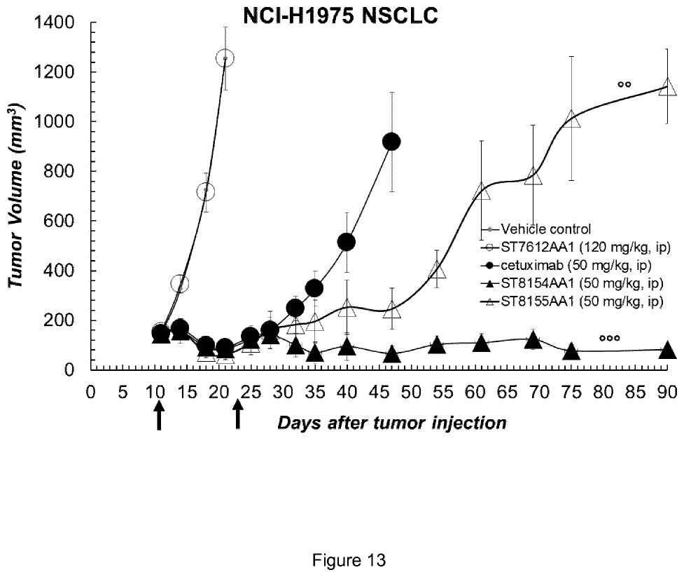

Results showed that the ADCs were significantly more efficacious than Cetuximab (P<0.001 and P<0.01). In particular, ST8155AA1 was more able to inhibit the Tumor Volume in comparison with Cetuximab of 77% (FIG. 13, Table 6), whereas ST8154AA1 was able to reduce the tumor growth of 95% with respect to Cetuximab (FIG. 13, Table 7). ST7612AA1, prodrug of the loaded toxin ST7464, when administered alone at 120 mg/kg, ip, q4dx4, was completely inactive, because the optimal schedule of an HDAC inhibitor is every day (qdx5/w). (FIG. 13).

Interestingly, the loaded dose of the drug ST8154AA1 and ST8155AA1 on mAb on mAb Cetuximab was around of 0.1 mg/kg.

TABLE 6
Antitumor efficacy of ST8155AA1 vs
Cetuximab against NCI-H1975 NSCLC.

| | | | Tumor or Metastases |
|---|---|---|---|
| | | | Inhibition (%) of |
| | | | ST8155AA1 vs Cetuximab |
| Tumor | Tumor | | at day 47 |
| histotype | implantation | Drug delivery | P value vs Cetuximab |
| NCI-H1975 | s.c. | ip, q4dx4 | 77, P < 0.01 |
| NSCLC | | | |

TABLE 7
Antitumor and antimetastatic efficacy of ST8154AA1
vs Cetuximab on different tumor models

| | | | Tumor or Metastases Inhibition |
|---|---|---|---|
| | | | (%) of ST8154AA1 and |
| Tumor | Tumor | | Cetuximab vs vehicle |
| histotype | implantation | Drug delivery | P value vs Cetuximab |
| NCI-H1975 | s.c. | i.p., q4dx4 | 95 (vs Cetuximab because |
| NSCLC | | | at day 90), P < 0.001 |
| A549 NSCLC | s.c. | i.p., q4dx4 | 60 vs 34, P < 0.05 |
| A549 NSCLC | i.v. | Aerosol q7dx4 | 90 vs 50, P < 0.01 |
| CAPAN-1 | Orthotopic | i.p., q4dx4 | 84 vs 49 and CR 6/10 vs |
| pancreas ca | in pancreas | | 2/10, P < 0.05 |
| PDX pancreas | s.c. | i.p., q4dx5 | 50 vs 25, P < 0.05 |
| ca. PA5363 | | | |

### Example 22: Determination of In Vivo Antitumor Effect of ST8154AA1 Given Ip Against A549 Non-Small Cell Lung Cancer

Nude mice were given a subcutaneous injection of 5×10⁶ A549 non-small cell lung carcinoma cells suspended in 100 μL cell culture medium RPMI supplemented with 10% FBS. Cetuximab and ST8154AA1 were administered intraperitoneally every 4 days for 4 days (q4dx4) at a dose of 50 mg/kg. Tumor measurements and data as in Example 21.

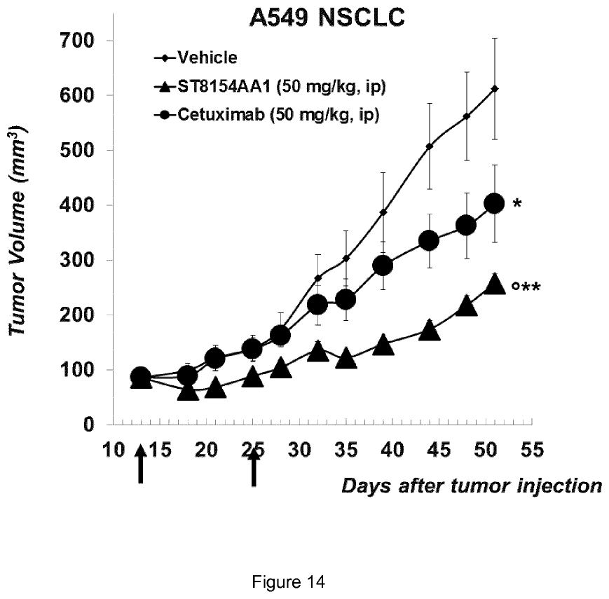

Treatment showed that ST8154AA1 was significantly more efficacious than Cetuximab (P<0.05) (FIG. 14, Table 7).

### Example 23: Determination of In Vivo Antitumor Effect of ST8154AA1 Delivered by Aerosol Against Metastatic A549 Non-Small Cell Lung Cancer

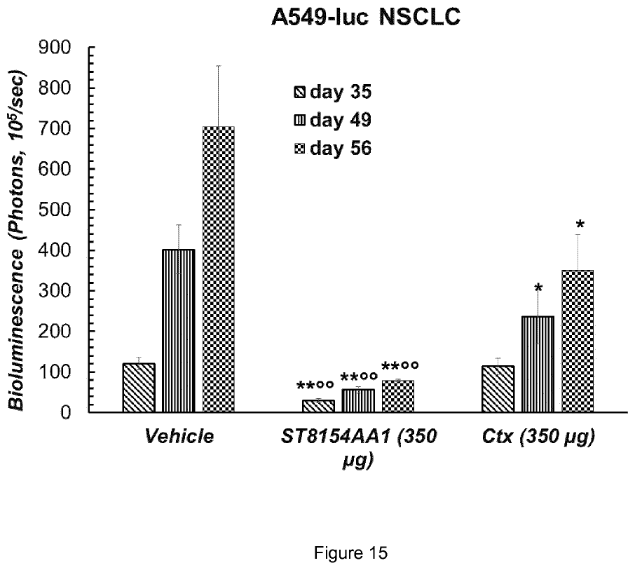

Metastatic lung cancer was established by injecting 5×10⁶ A549-luc-C8 (A549luc) cells into the tail vein of immunodeficient SCID/beige mice. After 1 week the mice were randomized and treated by whole body aerosol (by means of the AirFamily system, Pic indolor) with ADC or Cetuximab (3.5 mL of 100 μg/mL solution). Treatments were repeated according to the schedule q7dx4. Tumor bioluminescence imaging (BLI) was recorded at different time points by Xenogen IVIS Imaging System 200 (Perkin Elmer), 15 min after i.p. injection of luciferin (150 μg/mouse). The evaluation of bioluminescence showed that ADC was able to significantly inhibit tumor metastases with a higher potency in comparison with Cetuximab and at different times of tumor collection (P<0.01 and P<0.05) (FIG. 15, Table 7).

### Example 24: Determination of In Vivo Antitumor Effect of ST8154AA1 Delivered Ip Against CAPAN-1 Pancreas Cancer

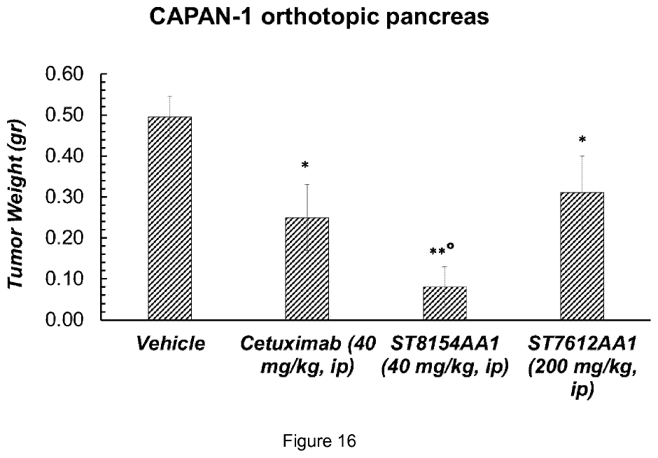

Comparison among ST8154AA1 (40 mg/kg, ip, q4dx4), ST7612AA1 (200 mg/kg, ip, q4dx4) and Cetuximab (40 mg/kg, ip, q4dx4) was evaluated in a CAPAN-1 orthotopic pancreatic tumor mouse model. Capan-1 (1×10⁶) cells were directly injected into pancreas of experimental groups. Treatments with ST8154AA1, ST7612AA1 and Cetuximab started 6 days after tumor inoculation and, 90 days after tumor injection mice were sacrificed to analyze the pancreas tumor weight. ST8154AA1 showed to inhibit the tumor growth of 84% with 6 out 10 mice with complete responses, while Cetuximab gave 49% of tumor growth inhibition with 2 out 10 mice with complete responses. ST7612AA1 alone showed a lower activity on tumor growth (32%), because q4dx4 is not the optimal schedule for am HDAC inhibitor. Tumor weight was evaluated 90 days after tumor injection and expressed as mean and SEM, P<0.05 vs Ctx) (FIG. 16, Table 7).

### Example 25: Determination of In Vivo Antitumor Effect of ST8154AA1 Delivered Ip Against a PDX (Patient-Derived Xenograft) Pancreas Cancer

NOD-SCID mice (from Jackson Laboratories) were given a single subcutaneous injection of cells of the patient PA5363 P2 at 51000 cells/100 μL re-suspended in an equal volume of Cultrex 10× spheroid phormation ECM.

Cetuximab and ST8154AA1 were administered intraperitoneally every 4 days for 5 days (q4dx5) at a dose of 40 mg/10 mL/kg. Tumor growth measurements and data as in Example 20.

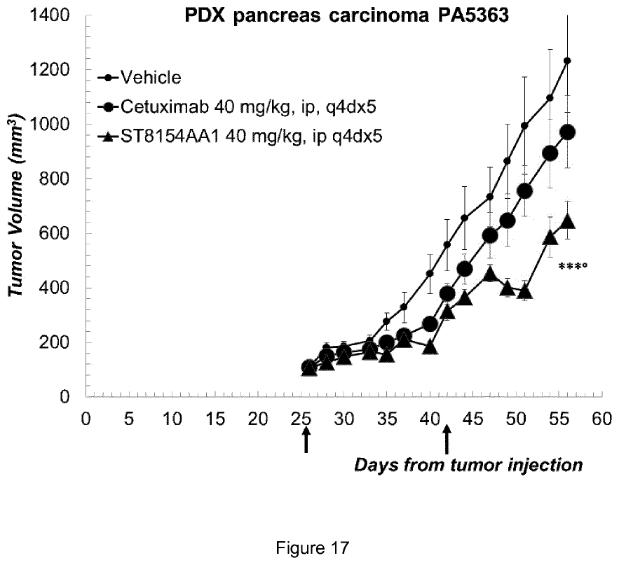

Treatment showed that the ST8154AA1 was significantly more efficacious than Cetuximab (P<0.05) (FIG. 17, Table 7).

### Example 26. Determination of In Vivo Antitumor Effect of ST8178AA1 and ST8176AA1 Given Ip Against SKOV-3 Ovarian Cancer

Nude Nu/Nu female mice (from Charles River, Italy) were given a single subcutaneous injection of 5×10⁶ SKOV-3 ovarian carcinoma cells suspended in 100 μL cell culture medium RPMI supplemented with 10% FBS.

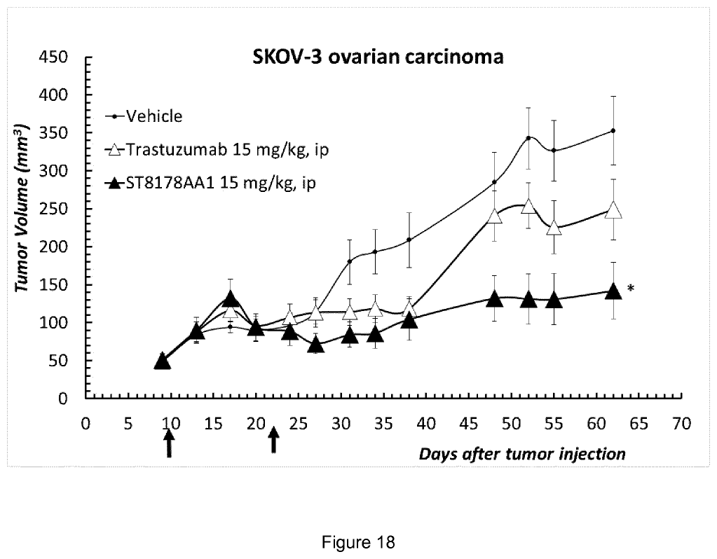

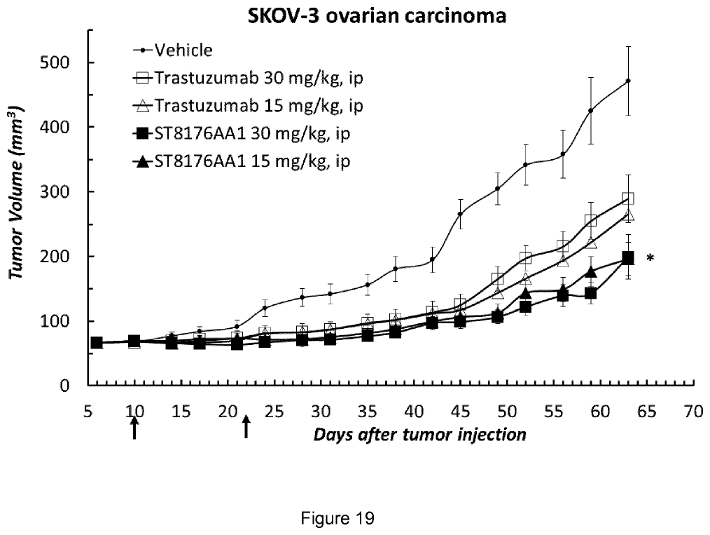

Mice for each experimental group were treated ip every four days, for 4 treatments (q4dx4), at a dose of 15 mg/10 mL/kg. Results showed that ST8178AA1 and ST8176AA1 were significantly more efficacious than Trastuzumab (P<0.05) (FIGS. 18-19, Tables 8 and 9). Tumor measurements and data as in Example 20. These data suggest a double effect of the ADC in comparison with Trastuzumab on the tumor growth.

TABLE 8
Antitumor efficacy of ST8178AA1 vs Trastuzumab
against SKOV-3 ovarian carcinoma

| | | | Tumor inhibition (%) |
|---|---|---|---|
| | | | of ST8176 and |
| Tumor | Tumor | | Trastuzumab vs vehicle |
| histotype | implantation | Drug delivery | P value vs Trastuzumab |
| SKOV-3 | s.c. | i.p., q4dx4 | 65 vs 40, P < 0.05 |
| ovarian ca. | | | |

### Example 27. Determination of In Vivo Antitumor Effect of ST8176AA1 Given Ip Against an Orthotopically Implanted SKOV-3 Ovarian Cancer

Nude Nu/Nu female mice (from Charles River, Italy) were given a single intraperitoneally injection of 10×10⁶ SKOV-3 ovarian carcinoma cells suspended in 200 μL cell culture medium RPMI supplemented with 10% FBS.

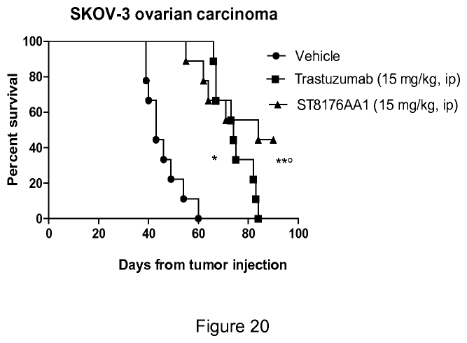

Mice were injected i.p. with either ST8176AA1, Trastuzumab (4 doses of 15 mg/kg once every 4 days) or vehicle (PBS) starting 3 days after tumor injection. Either Trastuzumab or ST8176AA1 were efficacious but the ADC also showed 4 out 9 mice cured after 90 days from tumor implantation (FIG. 20, Table 9).

TABLE 9
Antitumor and antimetastatic efficacy of ST8176AA1
vs Trastuzumab on different tumor models

| | | | Tumor inhibition (%) |
|---|---|---|---|
| | | | of ST8176 and |
| Tumor | Tumor | | Trastuzumab vs vehicle |
| histotype | implantation | Drug delivery | P value vs Trastuzumab |
| SKOV-3 | s.c. | i.p., q4dx4 | 65 vs 40, P < 0.05 |
| ovarian ca. | | | |
| SKOV-3 | orthotopic | i.p., q4dx4 | Increase in lifespan |
| ovarian ca. | | | 95 vs 72, P < 0.05 |
| LS174-T | s.c. | i.p., q4dx4 | 25 vs 0, P < 0.05 |
| colon ca. | | | |
| LS174-T | orthotopic | i.p., q4dx4 | Increase in lifespan |
| colon ca. | | | 35 vs 0, P < 0.05 |
| PDX pancreas | s.c. | i.p., q4dx5 | 35 vs 0, P < 0.05 |
| ca. PA5363 | | | |

### Example 28: Determination of In Vivo Anti-Tumor Efficacy of ST8176AA1 Given Ip Against an Intraperitoneal LS174-T Colon Cancer

The ADCs were also evaluated against an intraperitoneal tumor such as colon carcinoma, an aggressive tumor xenograft model.

The aim of this investigation was to demonstrate that ADCs may be also used by a local administration to treat diseases like peritoneal carcinomatosis. To demonstrate such activity LS-174T colon cancer cells were injected ip. The cells were collected and washed two times with PBS. Ten million cells were suspended in 0.2 mL of EMEM medium containing 20% of Matrigel™ and injected in the peritoneum of each mouse. All the treatments with the ADC were performed by i.p. injection at a volume of 200 μL 3 days post tumor injection, according to the schedule q4dx4.

Mice were monitored for mortality daily, while weight was recorded two times per week. Animals showing signs of discomfort, distress or in moribund condition were examined by the staff veterinarian or authorized personnel and, when necessary, humanely sacrificed to minimize undue pain or suffering.

Mortality data were processed according to the most appropriate statistical analysis to determine increase life span among the treatments and to produce a Kaplan-Mayer plot. All the statistical analysis was performed using the software GraphPad-Prism6.

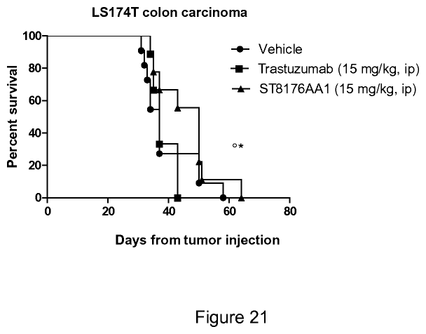

The results showed a median survival time (MST) of 37 days in both vehicle- and Trastuzumab-treated groups. By contrast the ADC ST8176AA1 revealed to significantly increase the median survival time to 50 days (P<0.05) (FIG. 21, Table 9).

Currently, the traditional treatment of peritoneal cancer consisting of systemic chemotherapy, with or without palliative surgery, shows poor effects in terms of outcome and presents pharmacological limitations in terms of poor drug distribution in the peritoneal cavity and penetration into peritoneal nodules (Oyais A 2014 Zentralbl Chir. 2014 141: 421-4).

By contrast, a recent innovative method named PIPAC (pressurized intraperitoneal aerosol chemotherapy) of local delivery has showed to enhance the efficacy of intraperitoneal chemotherapy (Solass W 2014 Ann Surg Oncol 21:553-9) and this procedure resulted to be safe, with no post-treatment renal or hepatic toxicity (Blanco 2014 20:2311-6; Solass 2013 Ann Surg Oncol 20: 3504-11).

Because the novel ADCs show an antitumor activity by aerosol and intraperitoneally on a local tumor, these data encourage to PIPAC use of the ADCs herein described in addition to the standard parenteral administration.

PIPAC (pressurized intraperitoneal aerosol chemotherapy) is an innovative method of local delivery that enhances the efficacy of intraperitoneal chemotherapy (Solass W 2014 Ann Surg Oncol 21:553-9).

This procedure resulted to be safe, with no post-treatment renal or hepatic toxicity (Blanco 2014 20:2311-6; Solass et al 2013 Ann Surg Oncol 20: 3504-11).

Differently, traditional treatment of peritoneal cancer consists of systemic chemotherapy, with or without palliative surgery, with poor effects in terms of outcome. Another problem is the pharmacological limitation in terms of poor drug distribution in the peritoneal cavity and penetration into peritoneal nodules (Oyais A et al 2014 Zentralbl Chir. 2014 141: 421-4). Based on biochemical analysis confirming stability of ADCs herein described upon nebulization and efficacy data on intraperitoneal delivery, the PIPAC use of ADCs is also justified.

### Example 29: Determination of In Vivo Antitumor Effect of ST8176AA1 Given Ip Against LS-174T Colon Cancer

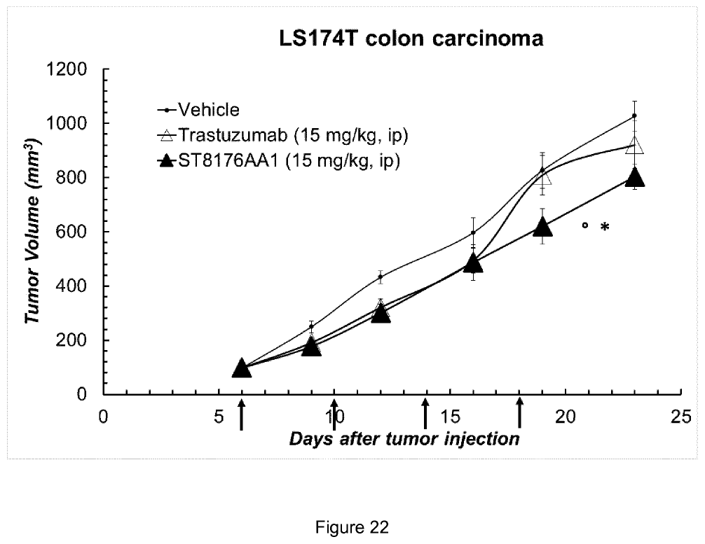

Tumors were allowed to develop in Nu/Nu mice for 6 days after s.c. injection of 5×10⁶ LS174T colon carcinoma cells. Lesion development and response to antibody treatment was monitored using a digital caliper. Mice were injected i.p. with either ST8176AA1, Trastuzumab (4 doses of 15 mg/kg once every 4 days) or vehicle (PBS). The ADC ST8176AA1 revealed to significantly inhibit the tumor growth of colon cancer (P<0.05 vs Trastuzumab) (FIG. 22, Table 9).

### Example 30: Dermination of In Vivo Antitumor Activity of ST8176AA1 Delivered Ip Against a PDX Pancreas Cancer

NOD-SCID mice (from Jackson Laboratories) were given a single subcutaneous injection of cells of the patient PA5363 P2 at 77000 cells/100 μL resuspended in an equal volume of Cultrex 10× spheroid phormation ECM.

Trastuzumab and ST8176AA1 were administered intraperitoneally every 4 days for 5 days (q4dx5) at a dose of 15 mg/ /kg in experimental groups of 10 mice for each group. Tumor measurements and data as in Example 20.

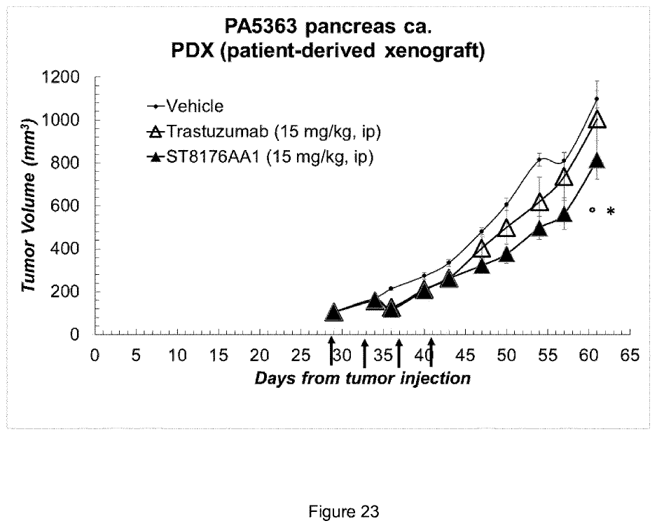

Treatment showed that the ST8176AA1 was significantly more efficacious than trastuzumab (P<0.05) and vs vehicle-treated group (P<0.05) (FIG. 23, Table 9).

### Example 31: Determination of In Vivo Antitumor Effect of the Combination of Cetuximab with a Low Dose of ST7612AA1, Corresponding to the Loaded Dose to mAb, Against A549 Lung Cancer

The anti-tumor effect of the ADC ST8154AA1 in comparison with the equimolar combination or Cetuximab and ST7612AA1 was also assessed against a model of A549 non-small cell lung carcinoma. Optimal doses of ST7612AA1 or Cetuximab were also investigated.

The human A549 lung carcinoma cells were cultured in their appropriate complete medium. On the day of tumor injection, cells were harvested from subconfluent cultures by trypsinization, washed with PBS, suspended in PBS and subcutaneously injected into the right flank of immunodeficient mice (5×10⁶/100 μL). Treatments started when tumor size was around 80 mm³ and mice were randomized into the experimental groups (10 mice/group):

The administration of ST8154AA1 q4dx4, ip, induced tumor growth inhibition, as evaluated by calibration during the study and post-mortem tumor weight, while, the equimolar mixture of the ADC components, Cetuximab (40 mg/kg, ip, q4dx4) and HDACi (0.1 mg/kg, ip, q4dx4), was not effective (Table 10).

In this experiment, cetuximab alone (40 mg/kg, ip, q4dx4) did not show any anti-tumor activity.

The association of optimal doses of cetuximab (40 mg/kg, ip, q4dx4) with ST7612AA1 (40 mg/kg, po, qdx5/wx3w) showed a comparable antitumor effect to that of ST7612AA1 alone (40 mg/kg, po qdx5/wx3w).

ST7612AA1 at 40 mg/kg, po qdx5/wx3w revealed a similar antitumor activity to that of the ADC ST8154AA1, whereas ST7612AA1 at 0.1 mg/kg, ip q4dx4 was not effective (Table 10).

TABLE 10
Antitumor activity of the combination of ST7612AA1
with cetuximab against A549 NSCLC.

| | | TVI at day 50, | TVI at day 56, |
|---|---|---|---|
| | Dose (mg/kg), | P value vs | P value vs |
| Group | route, schedule | vehicle | vehicle |
| Vehicle | 0 | — | — |
| Cetuximab | 40, ip, q4dx4 | 0, P = 0.91 | 0, P = 0.79 |
| ST7612AA1 | 40, po, qdx5/wx3w | 33, P = 0.007 | 35, P = 0.0009 |
| Cet + | 40 ip + 40 po | 20, P = 0.11 | 27, P = 0.07 |
| ST7612AA1 | | | |
| ST7612AA1 | 0.1, ip, q4dx4 | 0, P = 0.95 | 0, P = 0.88 |
| Cet + | 40 ip + 0.1 ip | 9, P = 0.22 | 8, P = 0.55 |
| ST7612AA1 | | | |
| ST8154AA1 | 40, q4dx4 | 21, P = 0.05 | 20, P = 0.04 |

1. An antibody-drug-conjugate of formula (I)
D-(CU)ₘ-(S1)ₙ-L-(S2)ₒ-(CG)ₚ-Ab (Formula I)
or a pharmaceutically acceptable salt thereof, wherein
D is a histone deacetylase inhibitor drug, selected from thiol-based histone deacetylase inhibitors, hydroxamic acid-based histone deacetylase inhibitors or benzamid-based histone deacetylase inhibitors,
CU is a connecting unit, which may be absent or which is selected from 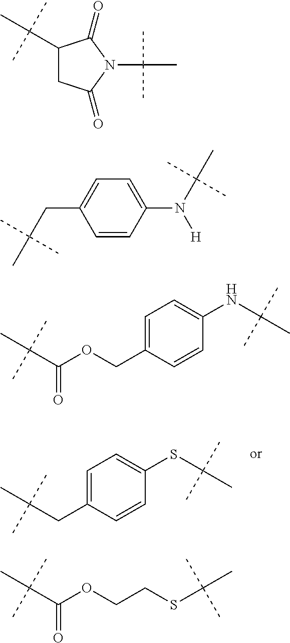
S1 is a spacer and may be absent or is 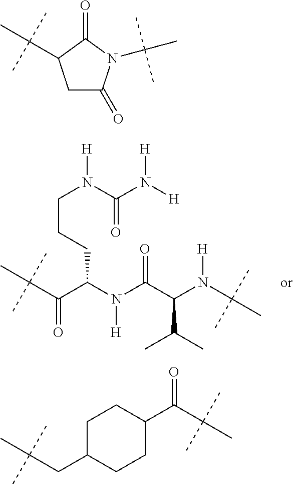
L is a linker, which is selected from (CH₂)q-CO, NH—(CH₂)r-(PEG)s-(CH₂)w-CO, NH—CO—(CH₂)r-(PEG)s-X—(CH₂)w-CO, wherein X may be absent, NH or O, q is an integer of 2 to 8, r may be absent or an integer of 1 to 4, s may be absent or an integer of 1 to 6, and w may be absent or an integer of 1 to 2,
S2 is a spacer and may be absent or is 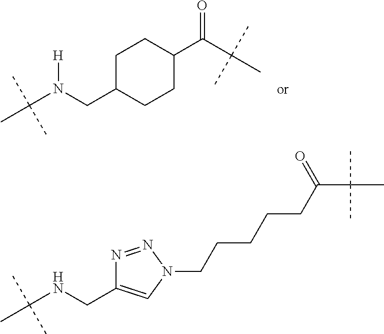
CG is a connecting group formed after conjugation to the cysteine thiol- or lysine amino-groups of the antibodies, which can be absent or one of following moieties: 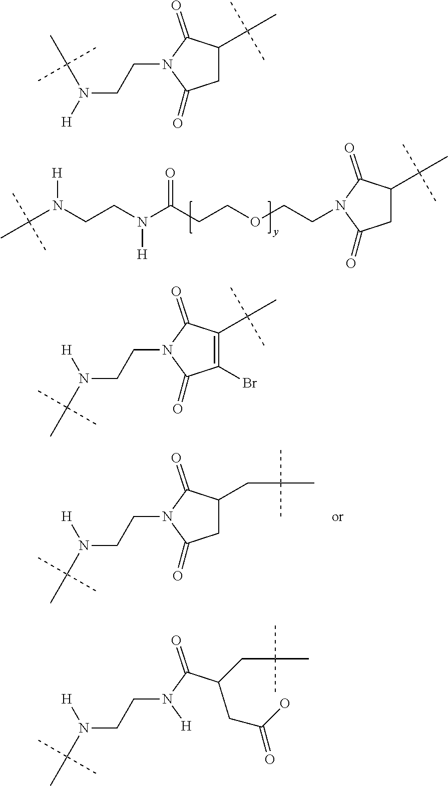
wherein y is an integer of 0 to 8
Ab is an antibody or an antigen binding fragment thereof, and
m, n, o and p represent integers of 0 or 1.

2. The antibody-drug conjugate according to claim 1, wherein the linker L is a moiety selected from 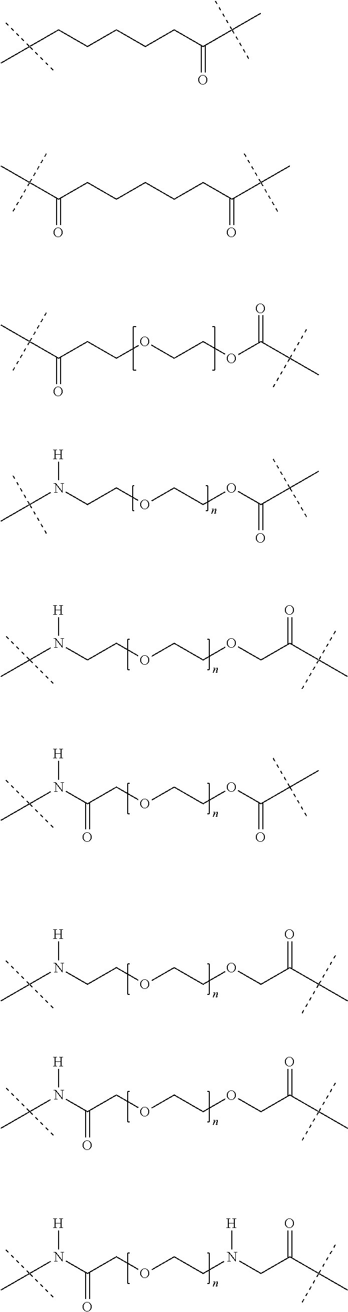 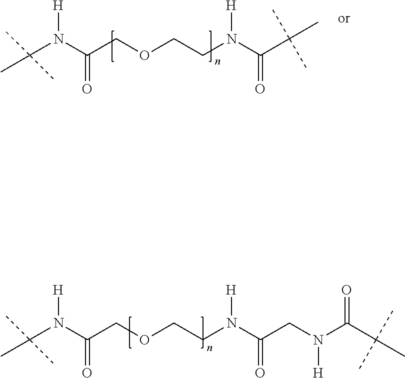
wherein n is an integer of 2 to 5.

3. The antibody-drug-conjugate according to claim 1, wherein the histone deacetylase inhibitor drug and the payload comprising the structure of D-(CU)ₘ-(S1)ₙ-L-(S2)ₒ-(CG)′ₚ- of Formula I is a compound selected from:

| I.D. | ST code | Chemical Structure | M.W. | Class |
|---|---|---|---|---|
| (1) | 8128AA1 | 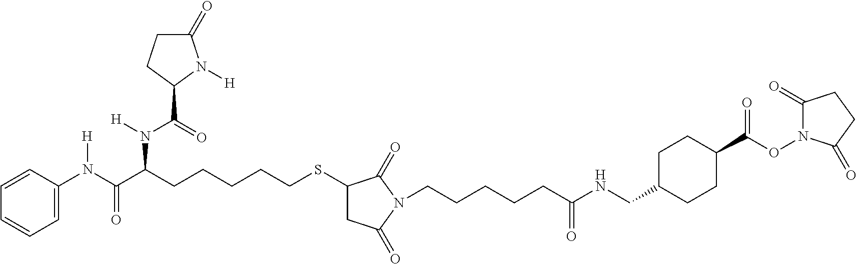 | 810.9 | NHS |
| (2) | 8152AA1 | 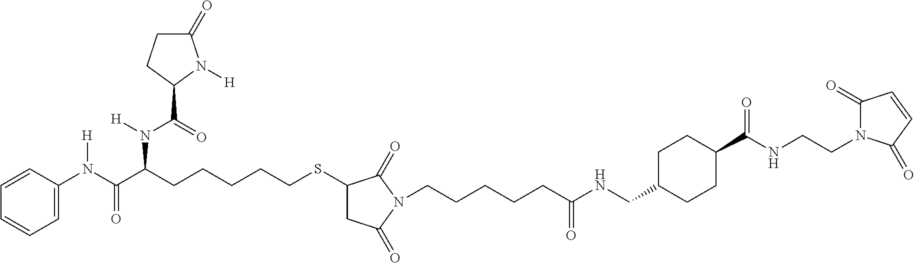 | 836.0 | Malei- mide |
| (3) | 8132AA1 | 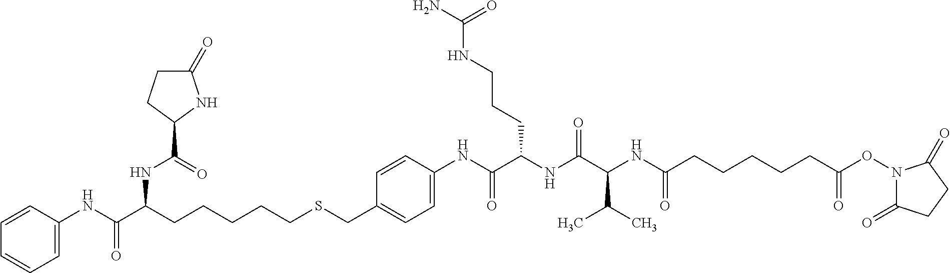 [SMILES: CC(C)[C@H](NC(=O)CCCCCC(=O)ON1C(=O)CCC1=O)C(=O)N[C@@H](CCCNC(N)=O)C(=O)Nc1ccc(CSCCCCC[C@H](NC(=O)[C@H]2CCC(=O)N2)C(=O)Nc2ccccc2)cc1] | 964.1 | NHS |
| (4) | 8190AA1 | 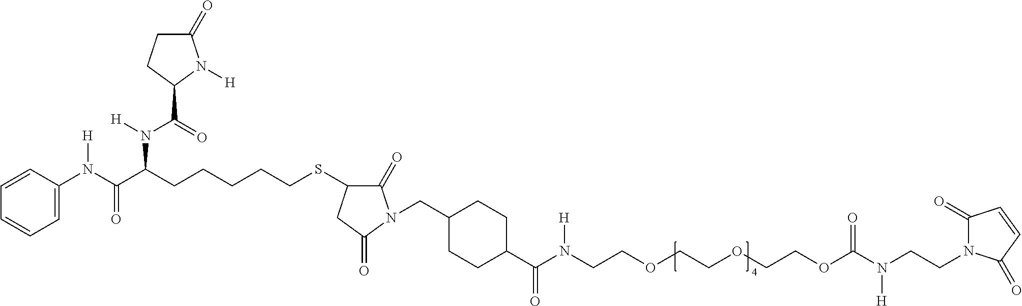 | 1030.2 | Malei- mide |
| (5) | 8189AA1 | 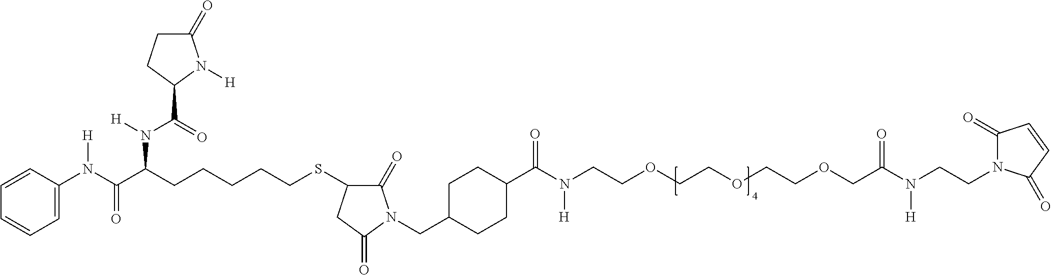 | 1044.2 | Malei- mide |
| (6) | 8191AA1 | 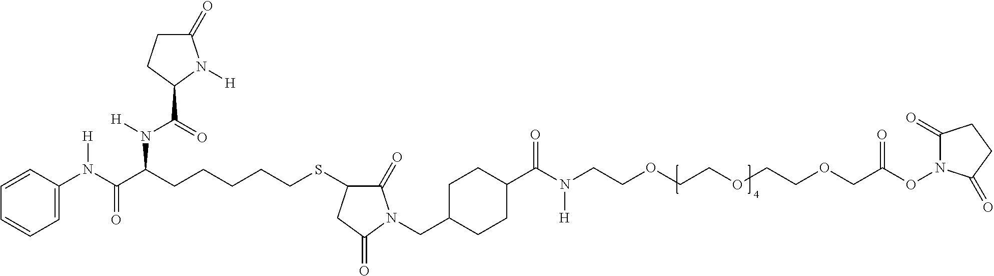 | 889.0 | NHS |
| (7) | 8197AA1 | 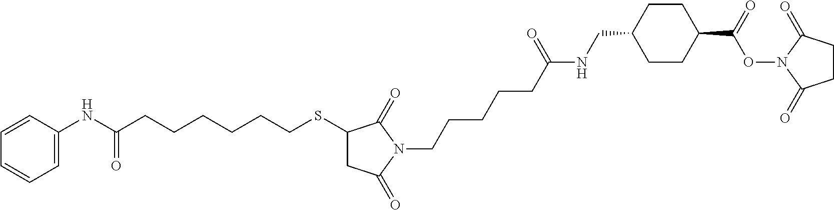 [SMILES: O=C(CCCCCN1C(=O)CC(SCCCCCCC(=O)Nc2ccccc2)C1=O)NC[C@H]1CC[C@H](C(=O)ON2C(=O)CCC2=O)CC1] | 684.8 | Malei- mide |
| (8) | 8235AA1 | 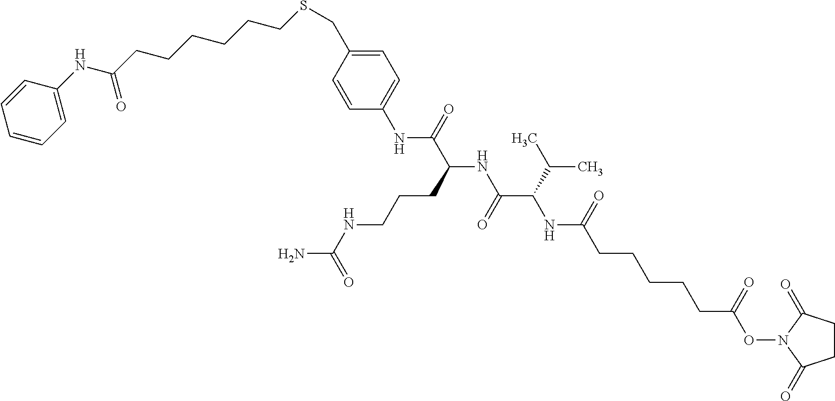 [SMILES: CC(C)[C@H](NC(=O)CCCCCC(=O)ON1C(=O)CCC1=O)C(=O)N[C@@H](CCCNC(N)=O)C(=O)Nc1ccc(CSCCCCCCC(=O)Nc2ccccc2)cc1] | 838.0 | NHS |
| (9) | 8217AA1 | 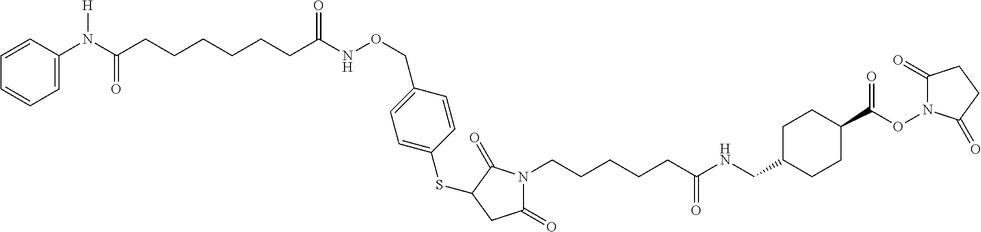 | 834.0 | NHS |
| (10) | 8201AA1 | 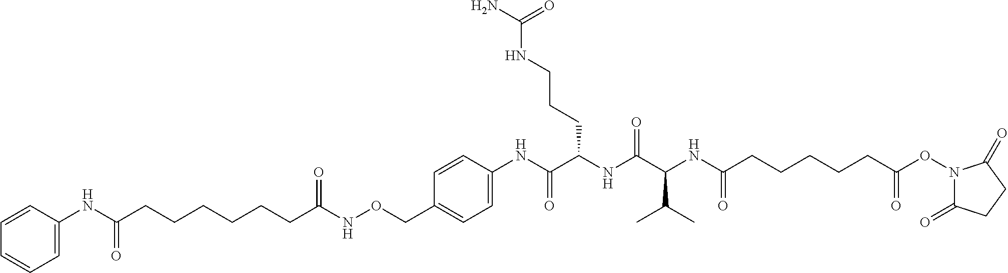 [SMILES: CC(C)[C@H](NC(=O)CCCCCC(=O)ON1C(=O)CCC1=O)C(=O)N[C@@H](CCCNC(N)=O)C(=O)Nc1ccc(CONC(=O)CCCCCCC(=O)Nc2ccccc2)cc1] | 865.0 | NHS |
| (11) | 8215AA1 | 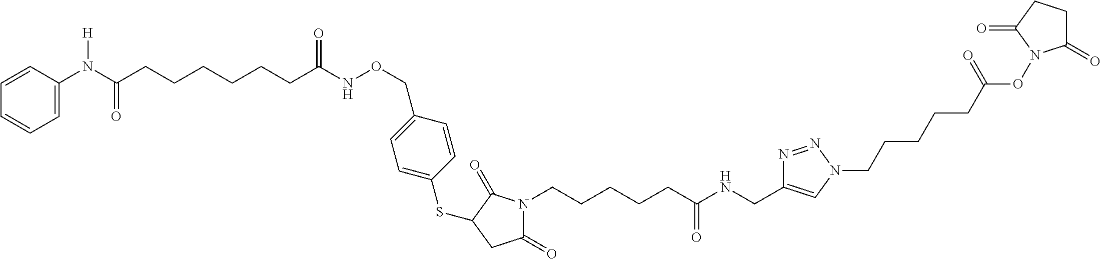 [SMILES: O=C(CCCCCN1C(=O)CC(Sc2ccc(CONC(=O)CCCCCCC(=O)Nc3ccccc3)cc2)C1=O)NCc1cn(CCCCCC(=O)ON2C(=O)CCC2=O)nn1] | 889.0 | NHS |
| (12) | 8216AA1 | 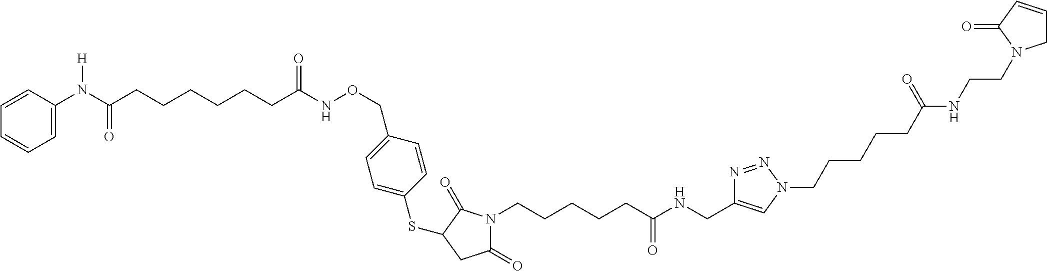 [SMILES: O=C(CCCCCn1cc(CNC(=O)CCCCCN2C(=O)CC(Sc3ccc(CONC(=O)CCCCCCC(=O)Nc4ccccc4)cc3)C2=O)nn1)NCCN1CC=CC1=O] | 914.1 | Malei- mide |
| (13) | 8236AA1 | 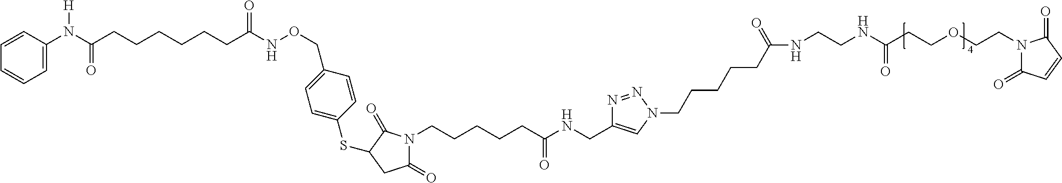 | 1161.4 | Malei- mide |
| (14) | 8232AA1 | 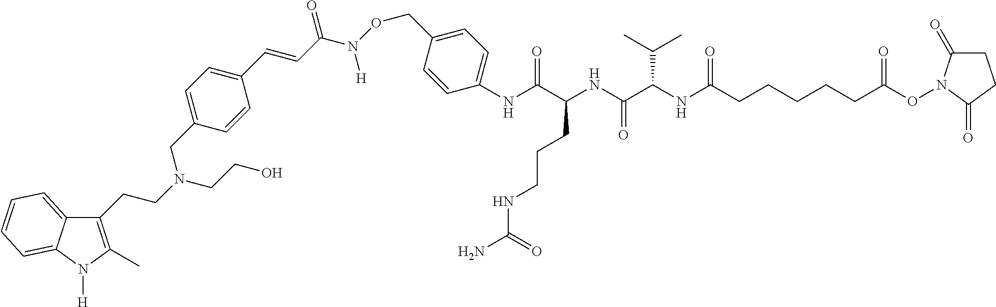 [SMILES: Cc1[nH]c2ccccc2c1CCN(CCO)Cc1ccc(/C=C/C(=O)NOCc2ccc(NC(=O)[C@H](CCCNC(N)=O)NC(=O)[C@@H](NC(=O)CCCCCC(=O)ON3C(=O)CCC3=O)C(C)C)cc2)cc1] | 994.1 | Malei- mide |
| (15) | 8233AA1 | 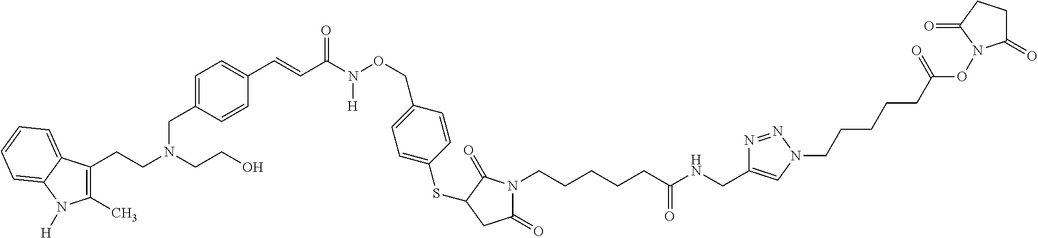 [SMILES: Cc1[nH]c2ccccc2c1CCN(CCO)Cc1ccc(/C=C/C(=O)NOCc2ccc(SC3CC(=O)N(CCCCCC(=O)NCc4cn(CCCCCC(=O)ON5C(=O)CCC5=O)nn4)C3=O)cc2)cc1] | 1018.2 | NHS |
| (16) | 8234AA1 | 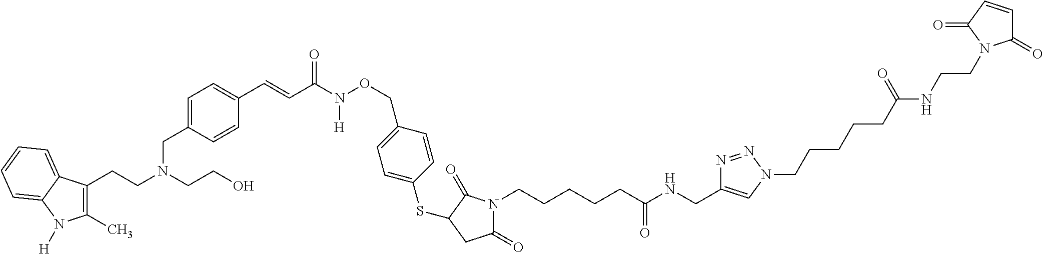 [SMILES: Cc1[nH]c2ccccc2c1CCN(CCO)Cc1ccc(/C=C/C(=O)NOCc2ccc(SC3CC(=O)N(CCCCCC(=O)NCc4cn(CCCCCC(=O)NCCN5C(=O)C=CC5=O)nn4)C3=O)cc2)cc1] | 1043.2 | Malei- mide |
| (17) | 8229AA1 | 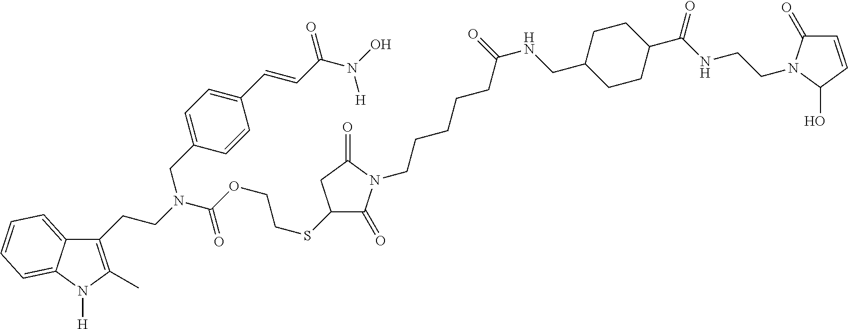 [SMILES: Cc1[nH]c2ccccc2c1CCN(Cc1ccc(/C=C/C(=O)NO)cc1)C(=O)OCCSC1CC(=O)N(CCCCCC(=O)NCC2CCC(C(=O)NCCN3C(=O)C=CC3O)CC2)C1=O] | 928.1 | Malei- mide |
| (18) | 8230AA1 | 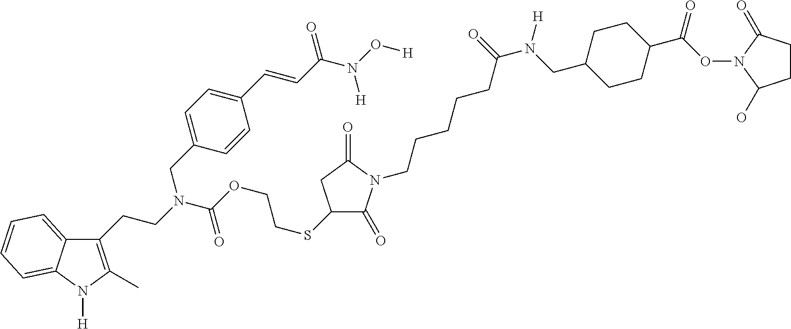 [SMILES: Cc1[nH]c2ccccc2c1CCN(Cc1ccc(/C=C/C(=O)NO)cc1)C(=O)OCCSC1CC(=O)N(CCCCCC(=O)NCC2CCC(C(=O)ON3C(=O)CCC3O)CC2)C1=O] | 903.0 | NHS |
| (19) | 8231AA1 | 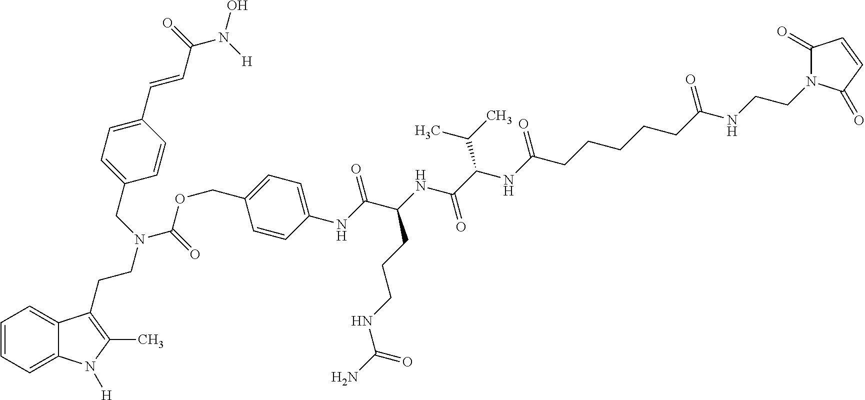 [SMILES: Cc1[nH]c2ccccc2c1CCN(Cc1ccc(/C=C/C(=O)NO)cc1)C(=O)OCc1ccc(NC(=O)[C@H](CCCNC(N)=O)NC(=O)[C@@H](NC(=O)CCCCCC(=O)NCCN2C(=O)C=CC2=O)C(C)C)cc1] | 1019.2 | Malei- mide |
| (20) | 8225AA1 | 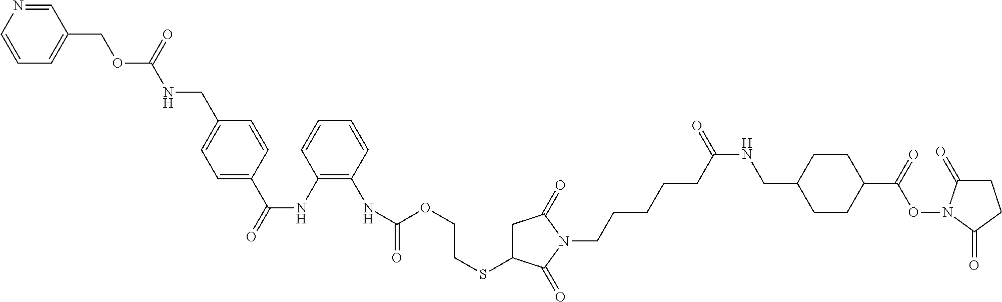 [SMILES: O=C(CCCCCN1C(=O)CC(SCCOC(=O)Nc2ccccc2NC(=O)c2ccc(CNC(=O)OCc3cccnc3)cc2)C1=O)NCC1CCC(C(=O)ON2C(=O)CCC2=O)CC1] | 928.0 | NHS |
| (21) | 8226AA1 | 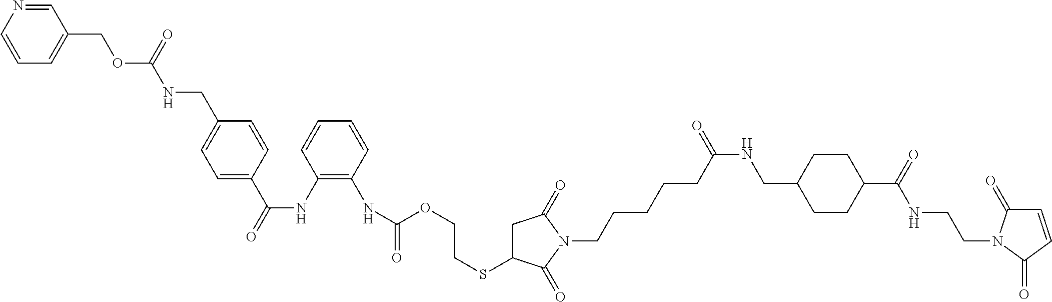 [SMILES: O=C(CCCCCN1C(=O)CC(SCCOC(=O)Nc2ccccc2NC(=O)c2ccc(CNC(=O)OCc3cccnc3)cc2)C1=O)NCC1CCC(C(=O)NCCN2C(=O)C=CC2=O)CC1] | 953.1 | Malei- mide |
| (22) | 8227AA1 | 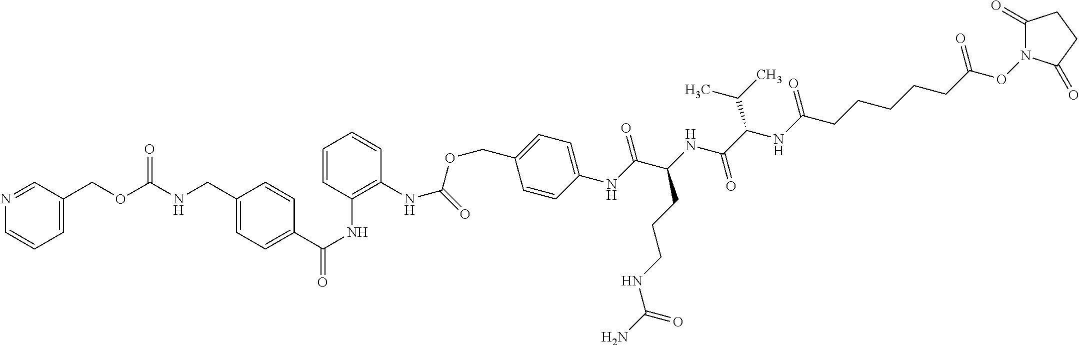 [SMILES: CC(C)[C@H](NC(=O)CCCCCC(=O)ON1C(=O)CCC1=O)C(=O)N[C@@H](CCCNC(N)=O)C(=O)Nc1ccc(COC(=O)Nc2ccccc2NC(=O)c2ccc(CNC(=O)OCc3cccnc3)cc2)cc1] | 1021.1 | NHS |
| (23) | 8228AA1 | 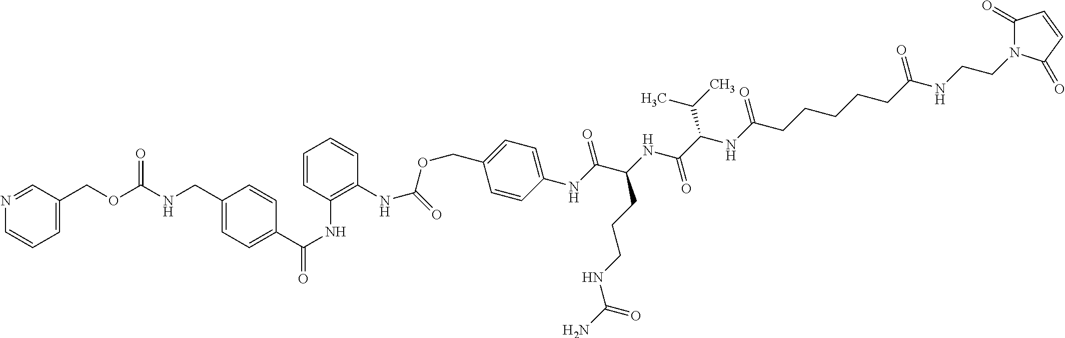 [SMILES: CC(C)[C@H](NC(=O)CCCCCC(=O)NCCN1C(=O)C=CC1=O)C(=O)N[C@@H](CCCNC(N)=O)C(=O)Nc1ccc(COC(=O)Nc2ccccc2NC(=O)c2ccc(CNC(=O)OCc3cccnc3)cc2)cc1] | 1046.1 | Malei- mide |

4. The antibody-drug conjugate according to claim 3, wherein the histone deacetylase inhibitor drug is a thiol-based inhibitor ST7464AA1 having the following formula 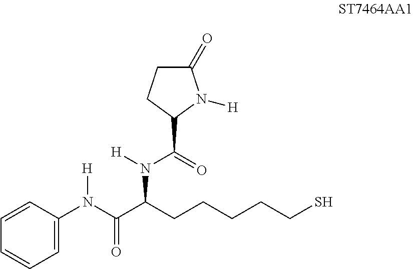
and the payload is a compound as evident from the following table:

| | | |
|---|---|---|
| 1 | 8128AA1 | 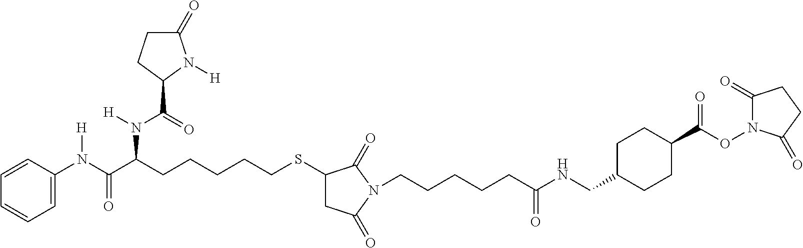 |
| 2 | 8152AA1 | 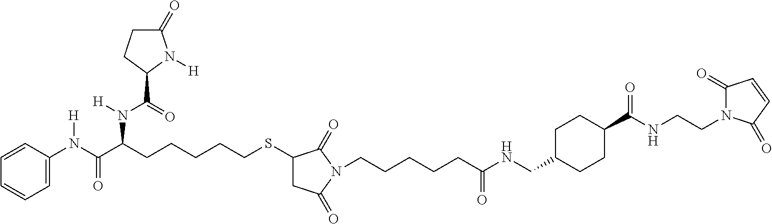 |
| 3 | 8132AA1 | 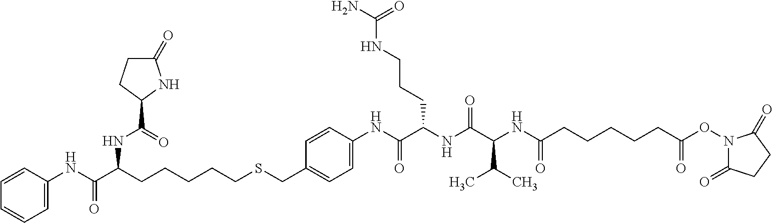 [SMILES: CC(C)[C@H](NC(=O)CCCCCC(=O)ON1C(=O)CCC1=O)C(=O)N[C@@H](CCCNC(N)=O)C(=O)Nc1ccc(CSCCCCC[C@H](NC(=O)[C@H]2CCC(=O)N2)C(=O)Nc2ccccc2)cc1] |
| 4 | 8190AA1 | 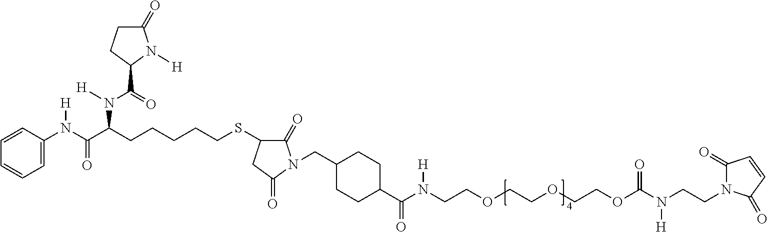 |

5. The antibody-drug-conjugate according to claim 1, wherein the connecting unit (CU) is selected from

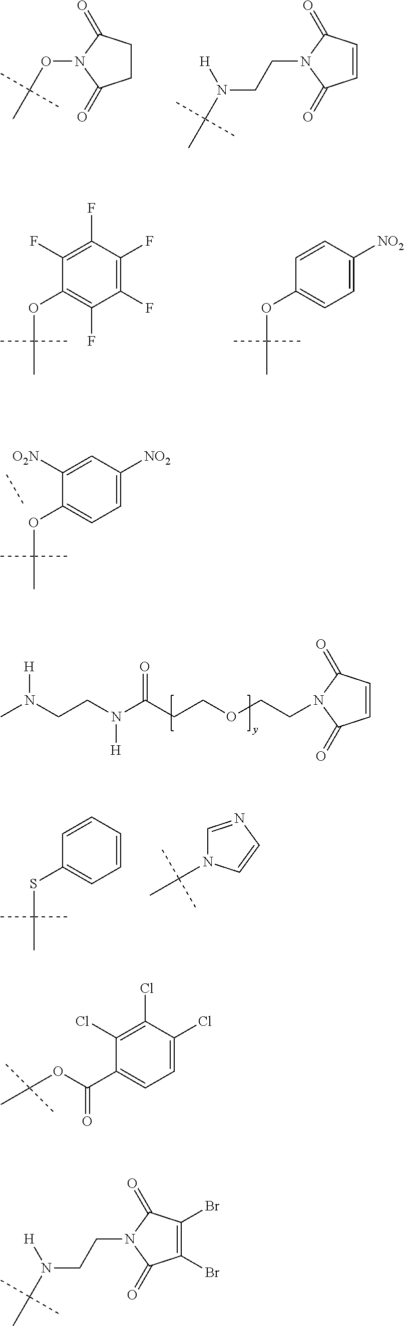

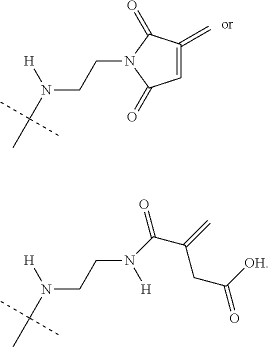

6. The antibody-drug conjugate according to one of claims 1 to 5, wherein the antibody is directed against a histone deacetylase inhibitor or against a receptor internalized by tumour cells to release a histone deacetylase inhibitor such as c-met or integrin receptors.

7. The antibody-drug-conjugate according to one of claims 1 to 6, wherein the antibody is an anti-EGFR family protein antibody.

8. The antibody-drug-conjugate according to one of claims 1 to 7, wherein the antibody is selected from Trastuzumab, Cetuximab, Bevacizumab, Panitumumab or related biosimilars.

9. An antibody-drug-conjugate according to claim 8 having a formula selected from

| I.D. ST code | Chemical Structure | SoC | DAR |
|---|---|---|---|
| (24) 8154AA1 | 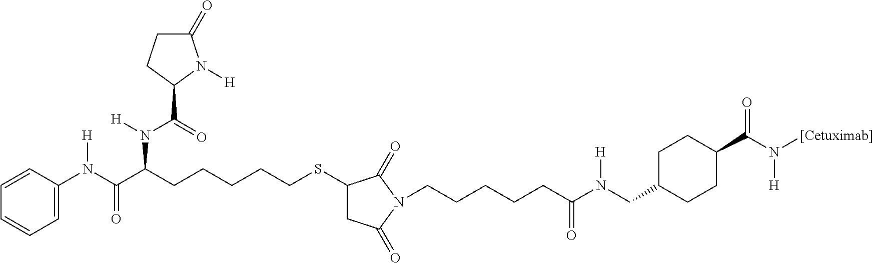 | Lys | 8.0 (±1) |
| (25) 8155AA1 | 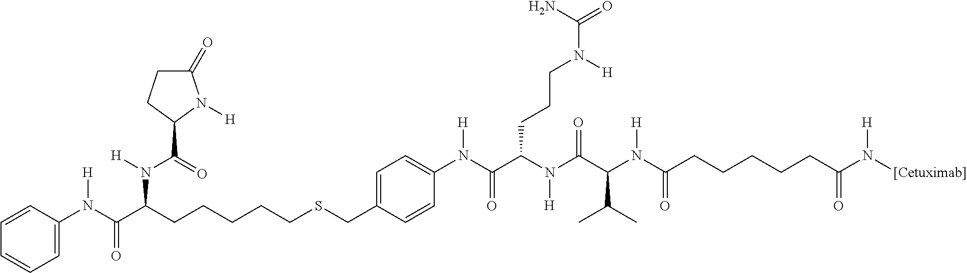 | Lys | 6.1 (±1) |
| (26) 8177AA1 | 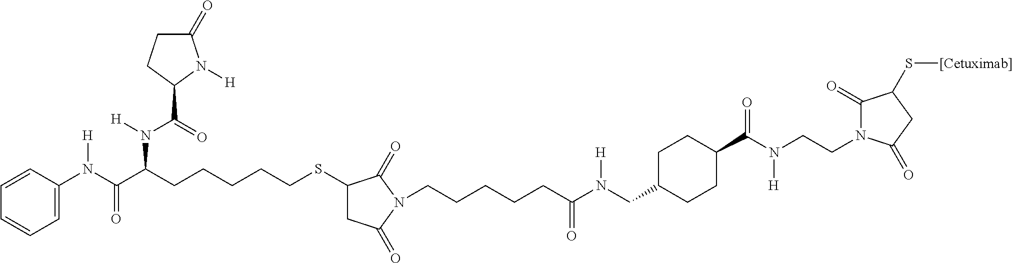 | Cys | 4.0 (±0.7) |
| (27) 8178AA1 | 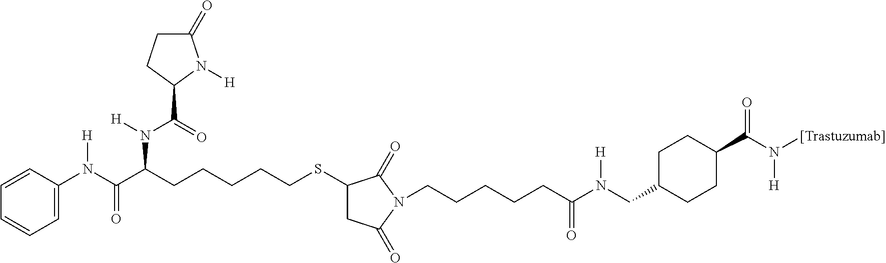 | Lys | 8.0 (±0.2) |
| (28) 8176AA1 | 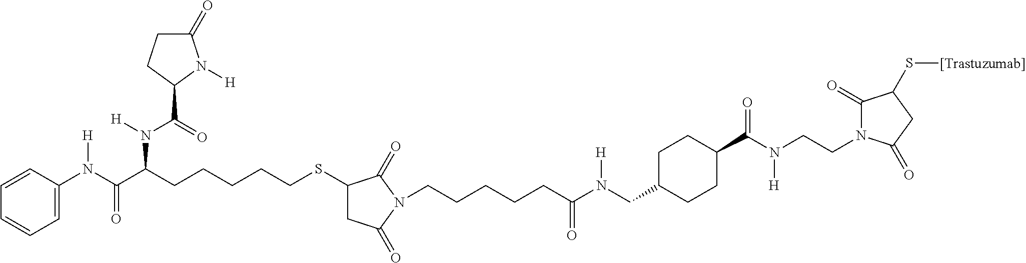 | Cys | 4.5 (±0.5) |
| (29) 8179AA1 | 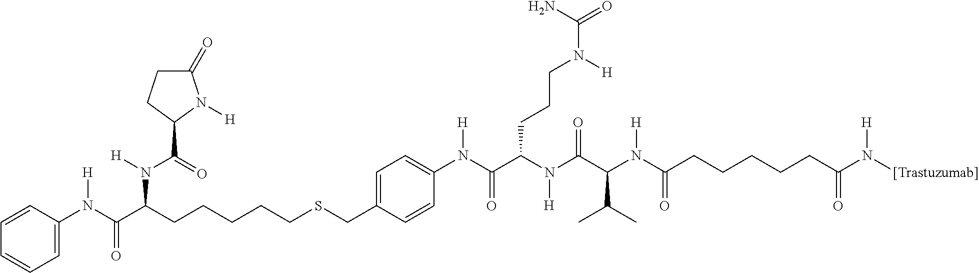 | Lys | 6.1 (±0.2) |
| (30) 8205AA1 | 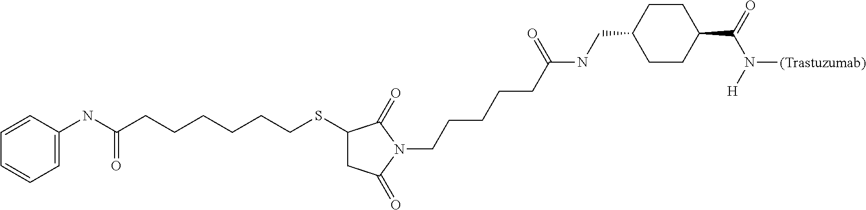 | Lys | 6.2 (±0.2) |
| (31) 8202AA1 | 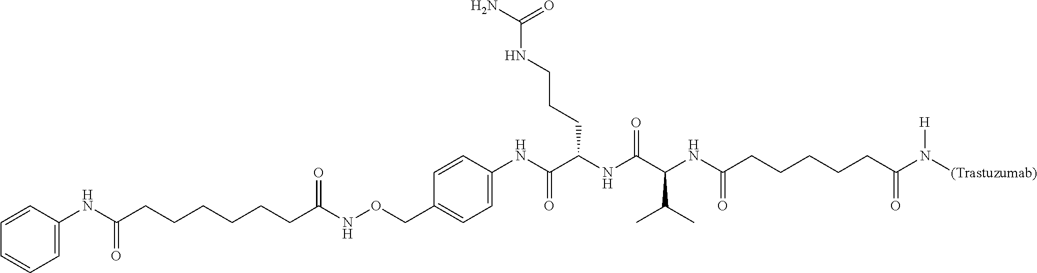 | Lys | 5.5 (±0.5) |
| (32) 8193AA1 | 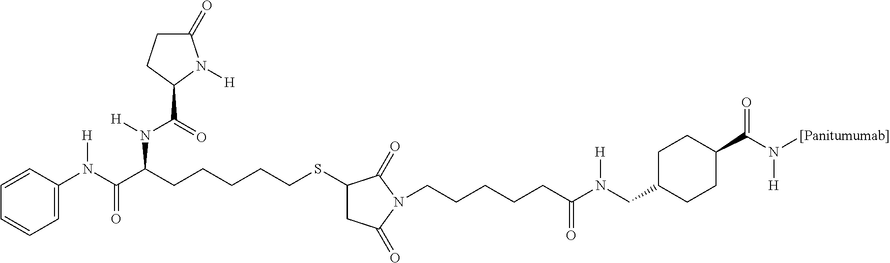 | Lys | 9.0 (±2) |
| (33) 8194AA1 | 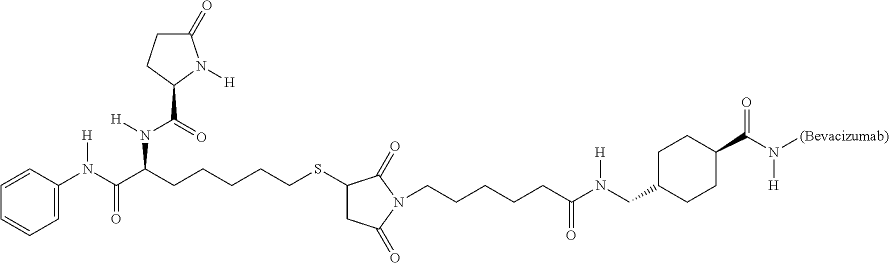 | Lys | 6.5 (±0.5) |
| (34) 8212AA1 | 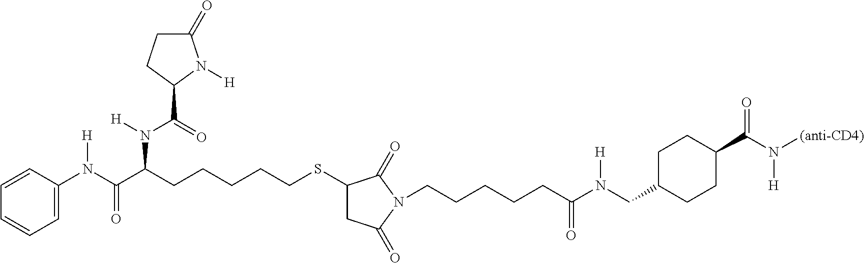 | Lys | 8.0 (±0.2) |
| (35) 8213AA1 | 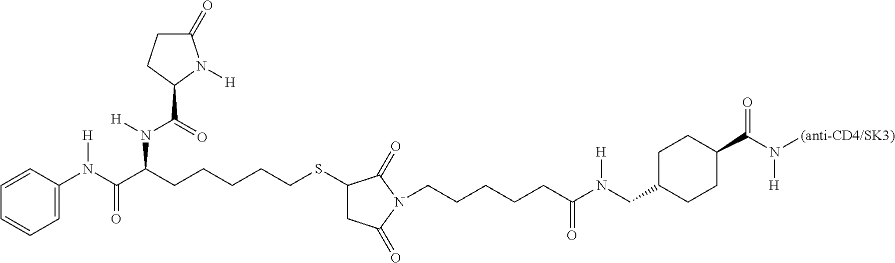 | Lys | 4.0 (±1) |
| (36) 8218AA1 | 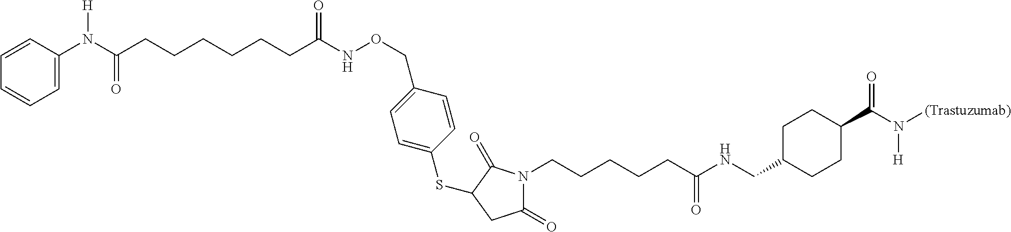 | Lys | 5.0 (±0.5) |
| (37) 8219AA1 | 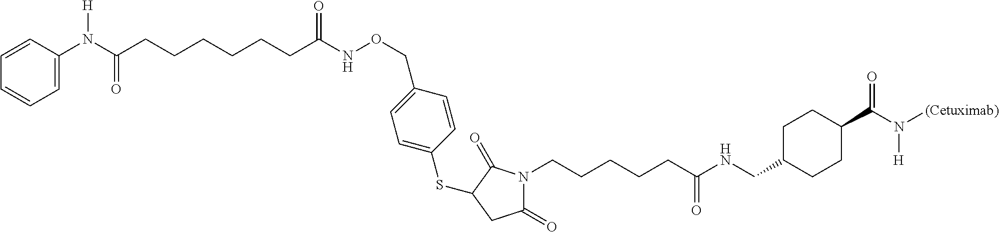 | Lys | 4.0 (±0.5) |

10. The antibody-drug-conjugate according to one of claims 1 to 9 for use as a medicament.

11. A pharmaceutical composition comprising an effective amount of the antibody-drug-conjugate according to one of claims 1 to 9 and a pharmaceutically acceptable excipient.

12. An antibody-drug-conjugate according to one of claims 1 to 9 or a pharmaceutical composition for use in the treatment of a cancer or a tumor expressing a receptor selected from ErbB1, ErbB2 or ErbB3.

13. The antibody-drug-conjugate according to one of claims 1 to 9 or the pharmaceutical composition for the use of claim 12, wherein the cancer is selected from lung, peritoneum, breast, colon, brain, head and neck, endometrial, cervix-endometrium, renal, pancreatic, gastric, colon, appendiceal, oesophageal, ovarian and prostate cancer; or from leukemia, pseudomyxoma peritonei, liver metastases and abdominal sarcoma of non-gut tissues.

14. The antibody-drug-conjugate according to one of claims 1 to 9 or the pharmaceutical composition for use as an adjuvant therapeutic in the treatment of HIV.

15. The antibody-drug-conjugate according to one of claims 1 to 13 or the pharmaceutical composition in a formulation suitable for local delivery by nebulization.

16. The antibody-drug-conjugate or the pharmaceutical composition according to claim 15 for use in the treatment of lung, breast, colon, brain, head and neck endometrial cancer, renal cancer, pancreatic cancer, gastric cancer, oesophageal cancer, ovarian and prostate cancer and leukaemia.
